SEQUENCE LISTING (48 sequences)

SEQ ID NO: 1 - DNA, 354 bp, synthetic construct
  misc_feature 1..354: note = Synthetic
  source 1..354: mol_type = other DNA
caggtgcagctggtggagtctgggggaggcttggtcaagcctggagggtccctgagactctcctgtgcagcctctggattcaccttcagtgactactccatgaactggatccgccaggctccagggaaggggctggagtggatctcatacattagttttagtggtaatagcatatactacgcagactctgtgaagggccgattcaccatctccagggacaacgccaagaactccctatatctgcaaatgaacagcctgagagtcgaggacacggccatatattactgtacgagccgggactggggatatgcttttgatatctggggccaagggacaatggtcaccgtctcttca

SEQ ID NO: 2 - protein, 118 aa, synthetic construct
  REGION 1..118: note = Synthetic
QVQLVESGGGLVKPGGSLRLSCAASGFTFSDYSMNWIRQAPGKGLEWISYISFSGNSIYYADSVKGRFTISRDNAKNSLYLQMNSLRVEDTAIYYCTSRDWGYAFDIWGQGTMVTVSS

SEQ ID NO: 3 - DNA, 24 bp, synthetic construct
  misc_feature 1..24: note = Synthetic
  source 1..24: mol_type = other DNA
ggattcaccttcagtgactactcc

SEQ ID NO: 4 - protein, 8 aa, synthetic construct
  REGION 1..8: note = Synthetic
GFTFSDYS

SEQ ID NO: 5 - DNA, 24 bp, synthetic construct
  misc_feature 1..24: note = Synthetic
  source 1..24: mol_type = other DNA
attagttttagtggtaatagcata

SEQ ID NO: 6 - protein, 8 aa, synthetic construct
  REGION 1..8: note = Synthetic
ISFSGNSI

SEQ ID NO: 7 - DNA, 33 bp, synthetic construct
  misc_feature 1..33: note = Synthetic
  source 1..33: mol_type = other DNA
acgagccgggactggggatatgcttttgatatc

SEQ ID NO: 8 - protein, 11 aa, synthetic construct
  REGION 1..11: note = Synthetic
TSRDWGYAFDI

SEQ ID NO: 9 - DNA, 321 bp, synthetic construct
  misc_feature 1..321: note = Synthetic
  source 1..321: mol_type = other DNA
gacatccagatgacccagtctccatccttcctgtctgcatctttaggagacagagtcaccatcacttgccaggcgagtcaggacattagcaattatttaaattggtatcaggtgaagccagggaaagcccctaagctcctgatctacgatgcatccaatttggaaacaggggtcccatcaaggttcagtggaagtggctctgggacagattttactttcaccatcagcagcctgcagcctgaagatattgcaacatatttctgtcaacaatatgataatctcccgtacatttttggccaggggaccaagctggagatcaaa

SEQ ID NO: 10 - protein, 107 aa, synthetic construct
  REGION 1..107: note = Synthetic
DIQMTQSPSFLSASLGDRVTITCQASQDISNYLNWYQVKPGKAPKLLIYDASNLETGVPSRFSGSGSGTDFTFTISSLQPEDIATYFCQQYDNLPYIFGQGTKLEIK

SEQ ID NO: 11 - DNA, 18 bp, synthetic construct
  misc_feature 1..18: note = Synthetic
  source 1..18: mol_type = other DNA
caggacattagcaattat

SEQ ID NO: 12 - protein, 6 aa, synthetic construct
  REGION 1..6: note = Synthetic
QDISNY

SEQ ID NO: 13 - protein, 9 aa, synthetic construct
  REGION 1..9: note = Synthetic
GATGCATCC

SEQ ID NO: 14 - skipped

SEQ ID NO: 15 - DNA, 27 bp, synthetic construct
  misc_feature 1..27: note = Synthetic
  source 1..27: mol_type = other DNA
caacaatatgataatctcccgtacatt

SEQ ID NO: 16 - protein, 9 aa, synthetic construct
  REGION 1..9: note = Synthetic
QQYDNLPYI

SEQ ID NO: 17 - DNA, 1338 bp, synthetic construct
  misc_feature 1..1338: note = Synthetic
  source 1..1338: mol_type = other DNA
caggtgcagctggtggagtctgggggaggcttggtcaagcctggagggtccctgagactctcctgtgcagcctctggattcaccttcagtgactactccatgaactggatccgccaggctccagggaaggggctggagtggatctcatacattagttttagtggtaatagcatatactacgcagactctgtgaagggccgattcaccatctccagggacaacgccaagaactccctatatctgcaaatgaacagcctgagagtcgaggacacggccatatattactgtacgagccgggactggggatatgcttttgatatctggggccaagggacaatggtcaccgtctcttcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 18 - protein, 445 aa, synthetic construct
  REGION 1..445: note = Synthetic
QVQLVESGGGLVKPGGSLRLSCAASGFTFSDYSMNWIRQAPGKGLEWISYISFSGNSIYYADSVKGRFTISRDNAKNSLYLQMNSLRVEDTAIYYCTSRDWGYAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 645 bp, synthetic construct
  misc_feature 1..645: note = Synthetic
  source 1..645: mol_type = other DNA
gacatccagatgacccagtctccatccttcctgtctgcatctttaggagacagagtcaccatcacttgccaggcgagtcaggacattagcaattatttaaattggtatcaggtgaagccagggaaagcccctaagctcctgatctacgatgcatccaatttggaaacaggggtcccatcaaggttcagtggaagtggctctgggacagattttactttcaccatcagcagcctgcagcctgaagatattgcaacatatttctgtcaacaatatgataatctcccgtacatttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 20 - protein, 214 aa, synthetic construct
  REGION 1..214: note = Synthetic
DIQMTQSPSFLSASLGDRVTITCQASQDISNYLNWYQVKPGKAPKLLIYDASNLETGVPSRFSGSGSGTDFTFTISSLQPEDIATYFCQQYDNLPYIFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 357 bp, synthetic construct
  misc_feature 1..357: note = Synthetic
  source 1..357: mol_type = other DNA
caggtgcagctggtggagtctgggggaggcgtggtccagcctgggaggtccctgagactctcctgtgcagcgtctggattcaccttcagtaactatgaaatgcactgggtccgccagactccaggcaaggggctggagtgggtggcacttatttggtatgatggaagtaataaatattctggagattccgtgaagggccgattcaccatttccagagacaattccaagagtacgctgtatctgcaaatgaacagcctgacagccgaggacacggctgtttattactgcaccggtataactggaactgaggatgcttttgatgtctggggccaagggacaatggtcaccgtctcttca

SEQ ID NO: 22 - protein, 119 aa, synthetic construct
  REGION 1..119: note = Synthetic
QVQLVESGGGVVQPGRSLRLSCAASGFTFSNYEMHWVRQTPGKGLEWVALIWYDGSNKYSGDSVKGRFTISRDNSKSTLYLQMNSLTAEDTAVYYCTGITGTEDAFDVWGQGTMVTVSS

SEQ ID NO: 23 - DNA, 24 bp, synthetic construct
  misc_feature 1..24: note = Synthetic
  source 1..24: mol_type = other DNA
ggattcaccttcagtaactatgaa

SEQ ID NO: 24 - protein, 8 aa, synthetic construct
  REGION 1..8: note = Synthetic
GFTFSNYE

SEQ ID NO: 25 - DNA, 24 bp, synthetic construct
  misc_feature 1..24: note = Synthetic
  source 1..24: mol_type = other DNA
atttggtatgatggaagtaataaa

SEQ ID NO: 26 - protein, 8 aa, synthetic construct
  REGION 1..8: note = Synthetic
IWYDGSNK

SEQ ID NO: 27 - DNA, 36 bp, synthetic construct
  misc_feature 1..36: note = Synthetic
  source 1..36: mol_type = other DNA
accggtataactggaactgaggatgcttttgatgtc

SEQ ID NO: 28 - protein, 12 aa, synthetic construct
  REGION 1..12: note = Synthetic
TGITGTEDAFDV

SEQ ID NO: 29 - DNA, 324 bp, synthetic construct
  misc_feature 1..324: note = Synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 30 - protein, 108 aa, synthetic construct
  REGION 1..108: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 31 - DNA, 18 bp, synthetic construct
  misc_feature 1..18: note = Synthetic
  source 1..18: mol_type = other DNA
cagagcattagcagctat

SEQ ID NO: 32 - protein, 6 aa, synthetic construct
  REGION 1..6: note = Synthetic
QSISSY

SEQ ID NO: 33-34 - skipped (2 entries)

SEQ ID NO: 35 - DNA, 30 bp, synthetic construct
  misc_feature 1..30: note = Synthetic
  source 1..30: mol_type = other DNA
caacagagttacagtacccctccgatcacc

SEQ ID NO: 36 - protein, 10 aa, synthetic construct
  REGION 1..10: note = Synthetic
QQSYSTPPIT

SEQ ID NO: 37 - DNA, 1341 bp, synthetic construct
  misc_feature 1..1341: note = Synthetic
  source 1..1341: mol_type = other DNA
caggtgcagctggtggagtctgggggaggcgtggtccagcctgggaggtccctgagactctcctgtgcagcgtctggattcaccttcagtaactatgaaatgcactgggtccgccagactccaggcaaggggctggagtgggtggcacttatttggtatgatggaagtaataaatattctggagattccgtgaagggccgattcaccatttccagagacaattccaagagtacgctgtatctgcaaatgaacagcctgacagccgaggacacggctgtttattactgcaccggtataactggaactgaggatgcttttgatgtctggggccaagggacaatggtcaccgtctcttcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 38 - protein, 446 aa, synthetic construct
  REGION 1..446: note = Synthetic
QVQLVESGGGVVQPGRSLRLSCAASGFTFSNYEMHWVRQTPGKGLEWVALIWYDGSNKYSGDSVKGRFTISRDNSKSTLYLQMNSLTAEDTAVYYCTGITGTEDAFDVWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - DNA, 648 bp, synthetic construct
  misc_feature 1..648: note = Synthetic
  source 1..648: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 40 - protein, 215 aa, synthetic construct
  REGION 1..215: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 625 aa, synthetic construct
  REGION 1..625: note = Synthetic
MIFLYQVVHFILFTSVSGECVTQLLKDTCFEGGDITTVFTPSAKYCQVVCTYHPRCLLFTFTAESPSEDPTRWFTCVLKDSVTETLPRVNRTAAISGYSFKQCSHQISACNKDIYVDLDMKGINYNSSVAKSAQECQERCTDDVHCHFFTYATRQFPSLEHRNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAV

SEQ ID NO: 42 - protein, 635 aa, synthetic construct
  REGION 1..635: note = Synthetic
ECVTQLLKDTCFEGGDITTVFTPSAKYCQVVCTYHPRCLLFTFTAESPSEDPTRWFTCVLKDSVTETLPRVNRTAAISGYSFKQCSHQISACNKDIYVDLDMKGINYNSSVAKSAQECQERCTDDVHCHFFTYATRQFPSLEHRNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAVEQKLISEEDLGGEQKLISEEDLHHHHHH

SEQ ID NO: 43 - protein, 634 aa, synthetic construct
  REGION 1..634: note = Synthetic
GCLTQLYENAFFRGGDVASMYTPNAQYCQMRCTFHPRCLLFSFLPASSINDMEKRFGCFLKDSVTGTLPKVHRTGAVSGHSLKQCHQISACNKDIYVDLDMKGINYNSSVAKSAQECQERCTDDVHCHFFTYATRQFPSLEHRNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAVEQKLISEEDLGGEQKLISEEDLHHHHHH

SEQ ID NO: 44 - protein, 633 aa, synthetic construct
  REGION 1..633: note = Synthetic
ECVTQLLKDTCFEGGDITTVFTPSAKYCQVVCTYHPRCLLFTFTAESPSEDPTRWFTCVLKDSVTETLPRVNRTAAISGYSFKQCSHQISCHRDIYKGVDMRGVNFNVSKVSSVEECQKRCTNNIRCQFFSYATQTFHKAEYRNNCLLKYSPGGTPTAIKVLSNVESGFSLKPCLSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAVEQKLISEEDLGGEQKLISEEDLHHHHHH

SEQ ID NO: 45 - protein, 633 aa, synthetic construct
  REGION 1..633: note = Synthetic
ECVTQLLKDTCFEGGDITTVFTPSAKYCQVVCTYHPRCLLFTFTAESPSEDPTRWFTCVLKDSVTETLPRVNRTAAISGYSFKQCSHQISACNKDIYVDLDMKGINYNSSVAKSAQECQERCTDDVHCHFFTYATRQFPSLEHRNICLLKHTQTGTPTRITKLDKVVSGFSLKSCALSNLCHMNIFQHLAFSDVDVARVLTPDAFVCRTICTYHPNCLFFTFYTNVWKIESQRNVCLLKTSESGTPSSSTPQENTISGYSLLTCHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAVEQKLISEEDLGGEQKLISEEDLHHHHHH

SEQ ID NO: 46 - protein, 633 aa, synthetic construct
  REGION 1..633: note = Synthetic
ECVTQLLKDTCFEGGDITTVFTPSAKYCQVVCTYHPRCLLFTFTAESPSEDPTRWFTCVLKDSVTETLPRVNRTAAISGYSFKQCSHQISACNKDIYVDLDMKGINYNSSVAKSAQECQERCTDDVHCHFFTYATRQFPSLEHRNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVCHSKIYPGVDFGGEELNVTFVKGVNVCQETCTKMIRCQFFTYSLLPEDCKEEKGKCFLRLSMDGSPTRIAYGTQGSSGYSLRLCMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAVEQKLISEEDLGGEQKLISEEDLHHHHHH

SEQ ID NO: 47 - protein, 647 aa, synthetic construct
  REGION 1..647: note = Synthetic
GCLTQLYENAFFRGGDVASMYTPNAQYCQMRCTFHPRCLLFSFLPASSINDMEKRFGCFLKDSVTGTLPKVHRTGAVSGHSLKQCGHQISACHRDIYKGVDMRGVNFNVSKVSSVEECQKRCTNNIRCQFFSYATQTFHKAEYRNNCLLKYSPGGTPTAIKVLSNVESGFSLKPCALSEIGCHMNIFQHLAFSDVDVARVLTPDAFVCRTICTYHPNCLFFTFYTNVWKIESQRNVCLLKTSESGTPSSSTPQENTISGYSLLTCKRTLPEPCHSKIYPGVDFGGEELNVTFVKGVNVCQETCTKMIRCQFFTYSLLPEDCKEEKCKCFLRLSMDGSPTRIAYGTQGSSGYSLRLCNTGDNSVCTTKTSTRIVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPICLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSDGKAQMQSPAEQKLISEEDLGGEQKLISEEDLHHHHHH

SEQ ID NO: 48 - protein, 638 aa, synthetic construct
  REGION 1..638: note = Synthetic
MILFKQATYFISLFATVSCGCLTQLYENAFFRGGDVASMYTPNAQYCQMRCTFHPRCLLFSFLPASSINDMEKRFGCFLKDSVTGTLPKVHRTGAVSGHSLKQCGHQISACHRDIYKGVDMRGVNFNVSKVSSVEECQKRCTNNIRCQFFSYATQTFHKAEYRNNCLLKYSPGGTPTAIKVLSNVESGFSLKPCALSEIGCHMNIFQHLAFSDVDVARVLTPDAFVCRTICTYHPNCLFFTFYTNVWKIESQRNVCLLKTSESGTPSSSTPQENTISGYSLLTCKRTLPEPCHSKIYPGVDFGGEELNVTFVKGVNVCQETCTKMIRCQFFTYSLLPEDCKEEKCKCFLRLSMDGSPTRIAYGTQGSSGYSLRLCNTGDNSVCTTKTSTRIVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPICLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSDGKAQMQSPA

(19) United States
(12) Patent Application Publication
(10) Pub. No.: US 2024/0190996 A1
(43) Pub. Date: Jun. 13, 2024

(54) FACTOR XI CATALYTIC DOMAIN-BINDING ANTIBODIES AND METHODS OF USE THEREOF

(71) Applicant: Regeneron Pharmaceuticals, Inc., Tarrytown, NY (US)

(72) Inventors: Dan Chalothorn, New York, NY (US); Lori C. Morton, Chappaqua, NY (US); KehDih Lai, Yardley, PA (US)

(21) Appl. No.: 18/387,658

(22) Filed: Nov. 7, 2023

Related U.S. Application Data
(60) Provisional application No. 63/423,272, filed on Nov. 7, 2022.

(51) Int. Cl. C07K 16/36; A61K 39/00; A61P 7/02; C12N 15/63

(52) U.S. Cl. CPC C07K 16/36; A61P 7/02; C12N 15/63; A61K 2039/505; C07K 2317/565; C07K 2317/92; C07K 2317/94

(57) ABSTRACT

The present disclosure provides antibodies that bind to the catalytic domain of Factor XI (FXI) and methods of using the same. According to certain embodiments, the antibodies are antagonist antibodies that inhibit blood clot formation via the intrinsic pathway without affecting hemostasis, as shown by their effect on prolonging aPTT without affecting PT. As such, these antagonist antibodies may be used to treat blood clotting diseases or disorders or treatment regimens that have clot formation as a risk factor, such as, but not limited to atrial fibrillation. In certain embodiments, the disclosure includes antibodies that bind FXI and mediate clot formation or thrombogenesis. The antibodies of the disclosure may be fully human, non-naturally occurring antibodies.

### RELATED APPLICATIONS

The instant application claims priority to U.S. Provisional Application No. 63/423,272, filed on Nov. 7, 2022. The entire contents of the foregoing application are expressly incorporated herein by reference.

### SEQUENCE LISTING

The instant application contains a Sequence Listing which has been submitted electronically in XML format and is hereby incorporated by reference in its entirety. Said XML copy, created on Jan. 5, 2024, is named 118003-01102.XML and is 2,596 bytes in size.

### FIELD

The present disclosure relates to antibodies that bind to the catalytic domain (CAT) of Factor XI (FXI), compositions comprising these antibodies, and methods of use thereof.

### BACKGROUND

The formation of blood clots (i.e., thrombi) is initiated either through (a) the contact pathway or through (b) the extrinsic pathway. Both pathways converge through (c) the common pathway to activate thrombin, which acts as a serine protease to convert soluble fibrinogen into insoluble strands of fibrin. Cross-linked fibrin protein is a major component of blood clots, along with aggregated platelets and red blood cells.

The extrinsic pathway moderates hemostasis at vascular injury. Here, exposed tissue factor (TF) activates factor VII (FVII) to form the FVIIa-TF complex, which activates factor X (FX) in the common pathway to generate prothrombinase, which generates thrombin and subsequent clot formation.

The contact pathway is distinct from the extrinsic pathway in that it is less involved in hemostasis but nonetheless effects clot formation. Here, coagulation is initiated by intrinsic events, such as the release of polyphosphate from platelets, or the extrusion of histone and DNA-laden neutrophil extracellular traps (NETs) from neutrophils, which activate factor XII (FXII). Activated FXII (i.e., FXIIa) further activates factor XI (FXI) to form FXIa, which leads to the generation of thrombin via the common pathway. Thrombin and platelet-produced polyphosphate also activate FXI in a feed-forward manner to amplify clot formation.

FXI is a zymogen of the plasma protease FXIa, which sustains thrombin generation via FIX activation. FXI is a 160 kDa disulfide-linked homodimer, in which each subunit consists of, from N-terminus to C-terminus, apple domains A1-A4 and a catalytic domain (referred to herein as “CAT” or “CD”). The disulfide bond is between the A4 domains of each subunit. FXI subunits are activated by cleavage of one or both of the Arg-Ile bonds located between the A4 and CAT domains to form FXIa. It is generally believed that cleavage of the Arg-Ile bond is catalyzed by FXIIa and/or thrombin.

### BRIEF SUMMARY

Provided herein are isolated monoclonal antibodies and antigen-binding fragments thereof that bind, e.g., specifically bind, to the CAT domain of Factor XI (FXI). In any of the embodiments disclosed herein, the antibody, or antigen-binding fragment thereof, may specifically bind the CAT domain of FXI. The isolated antibodies and antigen-binding fragments of the disclosure are useful for treating diseases and disorders associated with FXI activity or expression.

In its broadest aspect, the disclosure provides anti-FXI antibodies, which block FXI activity or activation and reduce blood clot formation. These antibodies may be used to prevent, treat, reduce the incidence of, or reduce the negative effects of blood clot formation in the blood stream or tissue in a patient in need thereof. Preferably, anti-FXI antibodies attenuate thrombosis without perturbing hemostasis.

In certain embodiments, the anti-FXI antibodies may be useful to treat various blood clotting disorders or diseases where the treatment the disease involves the use of anticoagulant therapy and where there is a risk to the patient of bleeding due to the use of anticoagulant therapy. Those indications, disorders or diseases include high risk atrial fibrillation, primary venous thromboembolism (VTE) prophylaxis, extended VTE treatment, prevention of recurrent ischemia after acute coronary syndrome, end-stage renal disease, medical devices (e.g., mechanical heart valves, ventricular assist devices, small caliber grafts, central venous catheters, and the like), extracorporeal circuits, and the like.

The antibodies of the disclosure can be full-length (for example, an IgG1 or IgG4 antibody) or may comprise only an antigen-binding portion (for example, a Fab, F(ab′)2 or scFv fragment), and may be modified to affect functionality, e.g., to eliminate residual effector functions (Reddy et al., 2000, J. Immunol. 164: 1925-1933).

An exemplary anti-FXI antibody of the present disclosure is listed in Tables 1A-1C herein. Tables 1A-1C set forth the amino acid sequences of exemplary heavy chain regions (HCs) and light chain regions (LCs) of the exemplary anti-FXI antibody. In one embodiment, the HC comprises a heavy chain variable region (HCVR) and the light chain comprises a light chain variable region (LCVR).

The present disclosure provides antibodies or antigen-binding fragments thereof that bind FXI, comprising an HC comprising an amino acid sequence selected from any of the HC amino acid sequences listed in Tables 1A-1C, or a substantially similar sequence thereof having at least 90%, at least 95%, at least 98% or at least 99% sequence identity thereto.

The present disclosure also provides antibodies or antigen-binding fragments thereof that bind FXI, comprising an LC comprising an amino acid sequence selected from any of the LC amino acid sequences listed in Tables 1A-1C, or a substantially similar sequence thereof having at least 90%, at least 95%, at least 98% or at least 99% sequence identity thereto.

The present disclosure also provides antibodies or antigen-binding fragments thereof that bind FXI, comprising an HCVR and an LCVR amino acid sequence pair (HCVR/LCVR) comprising any of the HCVR sequences of the HC listed in Tables 1A-1C paired with any of the LCVR amino acid sequences of the LC listed in Tables 1A-1C. According to certain embodiments, the present disclosure provides antibodies, or antigen-binding fragments thereof, comprising an HCVR/LCVR amino acid sequence pair contained within any of the exemplary anti-FXI antibodies listed in Tables 1A-1C.

Accordingly, in a first aspect, the disclosure provides an isolated antibody, or antigen-binding fragment thereof, that binds to serum clotting factor XI (FXI), wherein the antibody, or antigen-binding fragment thereof, comprises three heavy chain complementarity determining regions (CDRs) (HCDR1, HCDR2 and HCDR3) contained within a heavy chain variable region (HCVR), which is contained within a heavy chain region (HC) comprising an amino acid sequence as set forth in Tables 1A-1C, or a substantially similar sequence thereof having at least 90% sequence identity thereto; and three light chain CDRs (LCDR1, LCDR2 and LCDR3) contained within a light chain variable region (LCVR) which is contained within a light chain region (LC) comprising an amino acid sequence as set forth in Tables 1A-1C, or a substantially similar sequence thereof having at least 90% sequence identity thereto.

In one embodiment, the anti-FXI antibody, or antigen-binding fragment thereof, exhibits one or more properties selected from the group consisting of:
(a) is an antagonist antibody;
(b) binds human FXI with a K_{D} of less than about 5 pM as measured by surface plasmon resonance at 25° C. or at 37° C.;
(c) binds human FXIa with a K_{D} of less than about 300 pM as measured by surface plasmon resonance at 25° C. or at 37° C.;
(d) binds human FXI with a dissociative half-life (t½) of greater than about 1,000 minutes as measured by surface plasmon resonance at 25° C. or at 37° C.;
(e) binds human FXIa with a dissociative half-life (t½) of greater than about 95 minutes as measured by surface plasmon resonance at 25° C. or at 37° C.;
(f) inhibits activation of Factor Xa (FXa) by FXI in normal dilute plasma by at least about 85% to about 87% at an IC50 of less than about 39 pM to less than about 190 pM;
(g) inhibits activation of Factor Xa (FXa) by FXIa in normal dilute plasma by at least about 25% to about 35% at an IC50 of at least about 10 nM;
(h) preferentially binds to the CAT domain (i.e., catalytic domain) relative to full length FXI or any of the FXI domains such as the apple domain 2 (A2), PKA1, PKA3, or PKA4 as determined by label-free biolayer interferometry;
(i) competes for binding to FXI with an antibody that specifically binds to epitopes of within and overlapping with the FXI CAT domain;
(j) increases by at least 2, 2.5, 3, 3.5, 3.8 or 4-fold activated partial thromboplastin time (aPTT), which is a measure of intrinsic clotting time, in a primate sample in vitro without measurably affecting prothrombin time (PT), which is a measure of extrinsic clotting time;
(k) inhibits by 1%-6% intrinsic pathway peak thrombin activity in a primate sample in vitro;
(l) prolongs aPTT about two-fold in human plasma in vitro at a concentration of about ≤33 nM without doubling PT; and/or
m) inhibits intrinsic pathway thrombin production in human plasma in vitro at a concentration of about ≥20 nM with no effect on extrinsic pathway thrombin production with a dose up to about 500 nM.

In one embodiment, the disclosure provides an antibody, or antigen-binding fragment thereof, that binds Factor XI (FXI), wherein the antibody, or antigen-binding fragment thereof, comprises: (a) the complementarity determining regions (CDRs) of a heavy chain variable region (HCVR) comprising an amino acid sequence within the heavy chain region (HC) amino acid sequence as set forth in Tables 1A-1C; and (b) the CDRs of a light chain variable region (LCVR) comprising an amino acid sequence within the light chain region (LC) amino acid sequence as set forth in Tables 1A-1C.

In one embodiment, the antibody, or antigen-binding fragment thereof, that binds FXI comprises three heavy chain CDRs (HCDR1, HCDR2 and HCDR3) contained within a HC sequence of SEQ ID NO: 18, or a substantially similar sequence thereof having at least 90% sequence identity thereto; and three light chain CDRs (LCDR1, LCDR2 and LCDR3) contained within a LC sequence of SEQ ID NO: 20, or a substantially similar sequence thereof having at least 90% sequence identity thereto.

In one embodiment, the isolated antibody, or antigen-binding fragment thereof, that binds FXI comprises a HCVR having an amino acid sequence within the HC amino acid sequence provided in Tables 1A-1C below.

In one embodiment, the isolated antibody, or antigen-binding fragment thereof, that binds FXI further comprises a LCVR having an amino acid sequence within the LC amino acid sequence provided in Tables 1A-1C below.

In one embodiment, the isolated antibody, or antigen-binding fragment thereof, that binds FXI comprises a HCVR having an amino acid sequence within the HC amino acid sequence of provided in Tables 1A-1C below; and a LCVR having an amino acid sequence within the LC amino acid sequence provided in Tables 1A-1C below.

In one embodiment, the isolated antibody, or antigen-binding fragment thereof, that binds FXI comprises the CDRs of a HCVR/LCVR amino acid sequence pair provided within the HC/LC amino acid sequences in Tables 1A-1C below.

The present disclosure also provides antibodies or antigen-binding fragments thereof that bind FXI, comprising a heavy chain CDR1 (HCDR1) comprising an amino acid sequence comprised within the HC amino acid sequence listed in Tables 1A-1C or a substantially similar sequence thereof having at least 90%, at least 95%, at least 98% or at least 99% sequence identity thereto.

The present disclosure also provides antibodies or antigen-binding fragments thereof that bind FXI, comprising a heavy chain CDR2 (HCDR2) comprising an amino acid sequence comprised within the HC amino acid sequence listed in Tables 1A-1C or a substantially similar sequence thereof having at least 90%, at least 95%, at least 98% or at least 99% sequence identity thereto.

The present disclosure also provides antibodies or antigen-binding fragments thereof that bind FXI, comprising a heavy chain CDR3 (HCDR3) comprising an amino acid sequence comprised within the HC amino acid sequence listed in Tables 1A-1C or a substantially similar sequence thereof having at least 90%, at least 95%, at least 98% or at least 99% sequence identity thereto.

The present disclosure also provides antibodies or antigen-binding fragments thereof that bind FXI, comprising a light chain CDR1 (LCDR1) comprising an amino acid sequence comprised within the LC amino acid sequence listed in Tables 1A-1C or a substantially similar sequence thereof having at least 90%, at least 95%, at least 98% or at least 99% sequence identity thereto.

The present disclosure also provides antibodies or antigen-binding fragments thereof that bind FXI, comprising a light chain CDR2 (LCDR2) comprising an amino acid sequence comprised within the LC amino acid sequence listed in Tables 1A-1C or a substantially similar sequence thereof having at least 90%, at least 95%, at least 98% or at least 99% sequence identity thereto.

The present disclosure also provides antibodies or antigen-binding fragments thereof that bind FXI, comprising a light chain CDR3 (LCDR3) comprising an amino acid sequence comprised within the LC amino acid sequence listed in Tables 1A-1C or a substantially similar sequence thereof having at least 90%, at least 95%, at least 98% or at least 99% sequence identity thereto.

The present disclosure also provides antibodies or antigen-binding fragments thereof that bind FXI, comprising an HCDR3 and an LCDR3 amino acid sequence pair (HCDR3/LCDR3) comprising any of the HCDR3 amino acid sequences listed in Tables 1A-1C paired with any of the LCDR3 amino acid sequences listed in Tables 1A-1C. According to certain embodiments, the present disclosure provides antibodies, or antigen-binding fragments thereof, comprising an HCDR3/LCDR3 amino acid sequence pair contained within any of the exemplary anti-FXI antibodies listed in Tables 1A-1C. In certain embodiments, the HCDR3/LCDR3 amino acid sequence pair is SEQ ID Nos: 8/16.

The present disclosure also provides antibodies or antigen-binding fragments thereof that bind FXI, comprising a set of six CDRs (i.e., HCDR1, HCDR2, HCDR3, LCDR1, LCDR2, LCDR3) contained within any of the exemplary anti-FXI antibodies listed in Tables 1A-1C.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In one embodiment, the disclosure provides an isolated antibody, or antigen-binding fragment thereof, that binds FXI comprising:
(a) a HCDR1 domain having an amino acid sequence of SEQ ID NO: 4;
(b) a HCDR2 domain having an amino acid sequence of SEQ ID NO: 6;
(c) a HCDR3 domain having an amino acid sequence of SEQ ID NO: 8;
(d) a LCDR1 domain having an amino acid sequence of SEQ ID NO: 12;
(e) a LCDR2 domain having an amino acid sequence of SEQ ID NO: 14; and
(f) a LCDR3 domain having an amino acid sequence of SEQ ID NO: 16.

In one embodiment, the isolated antibody, or antigen-binding fragment thereof, comprises a set of six CDRs (HCDR1-HCDR2-HCDR3-LCDR1-LCDR2-LCDR3) of SEQ ID NOs: 4-6-8-12-14-16.

In one embodiment, the isolated antibody, or antigen-binding fragment thereof, that binds to FXI comprises an antibody, or antigen-binding fragment thereof, that competes for binding to FXI with a reference antibody, wherein the reference antibody preferentially binds to the catalytic domain of FXI.

In one embodiment, the isolated antibody, or antigen-binding fragment thereof, that binds to FXI comprises an antibody, or antigen-binding fragment thereof, that binds to the same epitope as a reference antibody, wherein the reference antibody preferentially binds to the catalytic domain of FXI.

In one embodiment, the isolated antibody, or antigen-binding fragment thereof, binds human FXI with a K_{D} of less than about 1,000 pM as measured by surface plasmon resonance at 25° C. or 37° C.

In one embodiment, the isolated antibody, or antigen-binding fragment thereof, binds human FXI with a K_{D} of less than about 500 pM as measured by surface plasmon resonance at 25° C. or 37° C.

In one embodiment, the isolated antibody, or antigen-binding fragment thereof, binds human FXI with a K_{D} of less than about 250 pM as measured by surface plasmon resonance at 25° C. or 37° C.

In one embodiment, the isolated antibody, or antigen-binding fragment thereof, binds human FXI with a K_{D} of less than about 100 pM as measured by surface plasmon resonance at 25° C. or 37° C.

In one embodiment, the isolated antibody, or antigen-binding fragment thereof, binds human FXI with a K_{D} of less than about 50 pM as measured by surface plasmon resonance at 25° C. or 37° C.

In one embodiment, the isolated antibody, or antigen-binding fragment thereof, binds human FXI with a K_{D} of less than about 25 pM as measured by surface plasmon resonance at 25° C. or 37° C.

In one embodiment, the isolated antibody, or antigen-binding fragment thereof, binds human FXI with a K_{D} of less than about 10 pM as measured by surface plasmon resonance at 25° C. or 37° C.

In one embodiment, the isolated antibody, or antigen-binding fragment thereof, binds human FXI with a K_{D} of less than about 5 pM as measured by surface plasmon resonance at 25° C. or 37° C.

In one embodiment, the isolated antibody, or antigen-binding fragment thereof, binds human FXI with a dissociative half-life (t½) of greater than about 10 minutes as measured by surface plasmon resonance at 25° C. or 37° C.

In one embodiment, the isolated antibody, or antigen-binding fragment thereof, binds human FXI with a t½ of greater than about 20 minutes as measured by surface plasmon resonance at 25° C. or 37° C.

In one embodiment, the isolated antibody, or antigen-binding fragment thereof, binds human FXI with a t½ of greater than about 60 minutes as measured by surface plasmon resonance at 25° C. or 37° C.

In one embodiment, the isolated antibody, or antigen-binding fragment thereof, binds human FXI with a t½ of greater than about 2 hours as measured by surface plasmon resonance at 25° C. or 37° C.

In one embodiment, the isolated antibody, or antigen-binding fragment thereof, binds human FXI with a t½ of greater than about 5 hours as measured by surface plasmon resonance at 25° C. or 37° C.

In one embodiment, the isolated antibody, or antigen-binding fragment thereof, binds human FXI with a t½ of greater than about 10 hours as measured by surface plasmon resonance at 25° C. or 37° C.

In one embodiment, the isolated antibody, or antigen-binding fragment thereof, binds human FXI with a t½ of greater than about 15 hours as measured by surface plasmon resonance at 25° C. or 37° C.

In one embodiment, the isolated antibody, or antigen-binding fragment thereof, binds human FXI with a t½ of greater than about 16 hours as measured by surface plasmon resonance at 25° C. or 37° C.

In one embodiment, the isolated antibody, or antigen-binding fragment thereof, binds human FXI with a t½ of greater than about 1,000 minutes as measured by surface plasmon resonance at 25° C. or 37° C.

In one embodiment, the isolated antibody, or antigen-binding fragment thereof, binds human FXIa with a K_{D} of less than about 100 nM as measured by surface plasmon resonance at 25° C. or 37° C.

In one embodiment, the isolated antibody, or antigen-binding fragment thereof, binds human FXIa with a K_{D} of less than about 10 nM as measured by surface plasmon resonance at 25° C. or 37° C.

In one embodiment, the isolated antibody, or antigen-binding fragment thereof, binds human FXIa with a K_{D} of less than about 1,000 pM as measured by surface plasmon resonance at 25° C. or 37° C.

In one embodiment, the isolated antibody, or antigen-binding fragment thereof, binds human FXIa with a K_{D} of less than about 500 pM as measured by surface plasmon resonance at 25° C. or 37° C.

In one embodiment, the isolated antibody, or antigen-binding fragment thereof, binds human FXIa with a K_{D} of less than about 300 PM as measured by surface plasmon resonance at 25° C. or 37° C.

In one embodiment, the isolated antibody, or antigen-binding fragment thereof, binds human FXIa with a dissociative half-life (t½) of greater than about 5 minutes as measured by surface plasmon resonance at 25° C. or 37° C.

In one embodiment, the isolated antibody, or antigen-binding fragment thereof, binds human FXIa with a t½ of greater than about 10 minutes as measured by surface plasmon resonance at 25° C. or 37° C.

In one embodiment, the isolated antibody, or antigen-binding fragment thereof, binds human FXI with a t½ of greater than about 15 minutes as measured by surface plasmon resonance at 25° C. or 37° C.

In one embodiment, the isolated antibody, or antigen-binding fragment thereof, binds human FXIa with a t½ of greater than about 20 minutes as measured by surface plasmon resonance at 25° C. or 37° C.

In one embodiment, the isolated antibody, or antigen-binding fragment thereof, binds human FXIa with a t½ of greater than about 25 minutes as measured by surface plasmon resonance at 25° C. or 37° C.

In one embodiment, the isolated antibody, or antigen-binding fragment thereof, binds human FXIa with a t½ of greater than about 50 minutes as measured by surface plasmon resonance at 25° C. or 37° C.

In one embodiment, the isolated antibody, or antigen-binding fragment thereof, binds human FXIa with a t½ of greater than about 75 minutes as measured by surface plasmon resonance at 25° C. or 37° C.

In one embodiment, the isolated antibody, or antigen-binding fragment thereof, binds human FXIa with a t½ of greater than about 95 minutes as measured by surface plasmon resonance at 25° C. or 37° C.

In one embodiment, the isolated antibody, or antigen-binding fragment thereof, that binds to FXI preferentially binds to the CAT domain (i.e., catalytic domain) relative to full length FXI, PKA1, PKA2, PKA3, and/or PKA4 as determined by label-free biolayer interferometry.

In one embodiment, the isolated antibody, or antigen-binding fragment thereof, that binds to FXI competes for binding to FXI with an antibody that specifically binds to epitopes of within and overlapping with the FXI CAT domain.

In one embodiment, the isolated antibody, or antigen-binding fragment thereof, that binds to FXI increases by at least 2.5-fold activated partial thromboplastin time (aPTT), which is a measure of intrinsic clotting time, in a primate in vitro without measurably affecting prothrombin time (PT), which is a measure of extrinsic clotting time.

In one embodiment, the isolated antibody, or antigen-binding fragment thereof, that binds to FXI inhibits intrinsic pathway peak thrombin activity in a primate in vitro by about 5%-15%, about 1%-20%, about 0.5%-25%, about 3%-5%, about 4%-6%, about 5%-7%, about 6%-8%, about 7%-9%, about 8%-10%, about 9%-11%, about 10%-12%, about 11%-13%, about 12%-14%, about 13%-15%, about 14%-16%, about 15%-17%, about 16%-18%, about 17%-19%, or about 18%-20%.

In one embodiment, the isolated antibody, or antigen-binding fragment thereof, that binds to FXI prolongs aPTT about two-fold in human plasma in vitro at a concentration of about 100 pM-100 nM, about 1 nM-50 nM, about 5 nM-40 nM, about 10 nM-35 nM, ≤60 nM, ≤55 nM, ≤50 nM, ≤45 nM, ≤40 nM, ≤39 nM, ≤38 nM, ≤37 nM, ≤36 nM, ≤35 nM, ≤34 nM, ≤33 nM, ≤32 nM, ≤31 nM, ≤30 nM, ≤25 nM, or ≤20 nM. Here, the anti-FXI prolongs aPTT about two-fold without doubling the PT.

In one embodiment, the isolated antibody, or antigen-binding fragment thereof, that binds to FXI inhibits intrinsic pathway thrombin production in human plasma in vitro at a concentration of about 10 nM-100 nM, about 15 nM-500 nM, about 20 nM-60 nM, about 25 nM-50 nM, ≥15 nM, ≥16 nM, ≥17 nM, ≥18 nM, ≥19 nM, ≥20 nM, ≥21 nM, ≥22 nM, ≥23 nM, ≥24 nM, ≥25 nM, ≥26 nM, ≥27 nM, ≥28 nM, ≥29 nM, ≥30 nM, ≥31 nM, ≥32 nM, ≥33 nM, ≥34 nM, ≥35 nM, ≥36 nM, ≥37 nM, ≥38 nM, ≥39 nM, or ≥40 nM. Here, the anti-FXI inhibits the intrinsic pathway thrombin production with no effect on extrinsic pathway thrombin production with a dose up to about 500 nM.

In a second aspect, the present disclosure provides nucleic acid molecules encoding anti-FXI antibodies or portions thereof. For example, the present disclosure provides nucleic acid molecules encoding any of the HCVR amino acid sequences listed in Tables 1A-1C; in certain embodiments the nucleic acid molecule comprises a polynucleotide sequence selected from any of the HCVR nucleic acid sequences listed in Tables 1A-1C, or a substantially similar sequence thereof having at least 90%, at least 95%, at least 98% or at least 99% sequence identity thereto.

The present disclosure also provides nucleic acid molecules encoding any of the LCVR amino acid sequences listed in Tables 1A-1C; in certain embodiments the nucleic acid molecule comprises a polynucleotide sequence selected from any of the LCVR nucleic acid sequences listed in Tables 1A-1C, or a substantially similar sequence thereof having at least 90%, at least 95%, at least 98% or at least 99% sequence identity thereto.

The present disclosure also provides nucleic acid molecules encoding any of the HCDR1 amino acid sequences listed in Tables 1A-1C; in certain embodiments the nucleic acid molecule comprises a polynucleotide sequence selected from any of the HCDR1 nucleic acid sequences listed in Tables 1A-1C, or a substantially similar sequence thereof having at least 90%, at least 95%, at least 98% or at least 99% sequence identity thereto.

The present disclosure also provides nucleic acid molecules encoding any of the HCDR2 amino acid sequences listed in Tables 1A-1C; in certain embodiments the nucleic acid molecule comprises a polynucleotide sequence selected from any of the HCDR2 nucleic acid sequences listed in Tables 1A-1C, or a substantially similar sequence thereof having at least 90%, at least 95%, at least 98% or at least 99% sequence identity thereto.

The present disclosure also provides nucleic acid molecules encoding any of the HCDR3 amino acid sequences listed in Tables 1A-1C; in certain embodiments the nucleic acid molecule comprises a polynucleotide sequence selected from any of the HCDR3 nucleic acid sequences listed in Tables 1A-1C, or a substantially similar sequence thereof having at least 90%, at least 95%, at least 98% or at least 99% sequence identity thereto.

The present disclosure also provides nucleic acid molecules encoding any of the LCDR1 amino acid sequences listed in Tables 1A-1C; in certain embodiments the nucleic acid molecule comprises a polynucleotide sequence selected from any of the LCDR1 nucleic acid sequences listed in Tables 1A-1C, or a substantially similar sequence thereof having at least 90%, at least 95%, at least 98% or at least 99% sequence identity thereto.

The present disclosure also provides nucleic acid molecules encoding any of the LCDR2 amino acid sequences listed in Tables 1A-1C; in certain embodiments the nucleic acid molecule comprises a polynucleotide sequence selected from any of the LCDR2 nucleic acid sequences listed in Tables 1A-1C, or a substantially similar sequence thereof having at least 90%, at least 95%, at least 98% or at least 99% sequence identity thereto.

The present disclosure also provides nucleic acid molecules encoding any of the LCDR3 amino acid sequences listed in Tables 1A-1C; in certain embodiments the nucleic acid molecule comprises a polynucleotide sequence selected from any of the LCDR3 nucleic acid sequences listed in Tables 1A-1C, or a substantially similar sequence thereof having at least 90%, at least 95%, at least 98% or at least 99% sequence identity thereto.

The present disclosure also provides nucleic acid molecules encoding an HCVR, wherein the HCVR comprises a set of three CDRs (i.e., HCDR1, HCDR2, HCDR3), wherein the HCDR1, HCDR2, HCDR3 amino acid sequence set is as defined by any of the exemplary anti-FXI antibodies listed in Tables 1A-1C.

The present disclosure also provides nucleic acid molecules encoding an LCVR, wherein the LCVR comprises a set of three CDRs (i.e., LCDR1, LCDR2, LCDR3), wherein the LCDR1, LCDR2, LCDR3 amino acid sequence set is as defined by any of the exemplary anti-FXI antibodies listed in Tables 1A-1C.

The present disclosure also provides nucleic acid molecules encoding both an HCVR and an LCVR, wherein the HCVR comprises an amino acid sequence of any of the HCVR amino acid sequences listed in Tables 1A-1C, and wherein the LCVR comprises an amino acid sequence of any of the LCVR amino acid sequences listed in Tables 1A-1C. In certain embodiments, the nucleic acid molecule comprises a polynucleotide sequence selected from any of the HCVR nucleic acid sequences listed in Tables 1A-1C, or a substantially similar sequence thereof having at least 90%, at least 95%, at least 98% or at least 99% sequence identity thereto, and a polynucleotide sequence selected from any of the LCVR nucleic acid sequences listed in Tables 1A-1C, or a substantially similar sequence thereof having at least 90%, at least 95%, at least 98% or at least 99% sequence identity thereto. In certain embodiments according to this aspect of the disclosure, the nucleic acid molecule encodes an HCVR and LCVR, wherein the HCVR and LCVR are both derived from the same anti-FXI antibody listed in Tables 1A-1C.

In a third aspect, the present disclosure provides recombinant expression vectors capable of expressing a polypeptide comprising a heavy or light chain variable region of an anti-FXI antibody. For example, the present disclosure includes recombinant expression vectors comprising any of the nucleic acid molecules mentioned above, i.e., nucleic acid molecules encoding any of the HCVR, LCVR, and/or CDR sequences as set forth in Tables 1A-1C. Also included within the scope of the present disclosure are host cells into which such vectors have been introduced, as well as methods of producing the antibodies or portions thereof by culturing the host cells under conditions permitting production of the antibodies or antibody fragments, and recovering the antibodies and antibody fragments so produced.

The present disclosure includes anti-FXI antibodies having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC).

In a fourth aspect, the disclosure provides a pharmaceutical composition comprising at least one antibody of the disclosure, or an antigen binding fragment thereof, which specifically binds FXI and a pharmaceutically acceptable carrier.

In a related aspect, the disclosure features a composition, which is a combination of an anti-FXI antibody and a second therapeutic agent. In one embodiment, the second therapeutic agent is any agent that is advantageously combined with an anti-FXI antibody. The second therapeutic agent may be useful for alleviating at least one symptom of the neurodegenerative disease or disorder.

In a fifth aspect, the disclosure provides a method for enhancing a biological activity mediated by FXI, the method comprising contacting FXI with a biologically effective amount of an antagonist anti-FXI antibody of Tables 1A-1C, or contacting FXI with a pharmaceutical composition containing a biologically effective amount of an antagonist anti-FXI antibody of Tables 1A-1C.

In certain embodiments, the biological activity is blood clotting or blood clotting as a result of the intrinsic clotting pathway and not blood clotting as a result of the extrinsic (i.e., e.g., tissue factor induced) pathway; and blood clotting or blood clotting as a result of the intrinsic clotting pathway and not blood clotting as a result of the extrinsic pathway is inhibited or otherwise reduced upon contact of FXI or FXIa with an antagonist anti-FXI antibody.

In a sixth aspect, the disclosure provides therapeutic methods for treating a disease or disorder associated with FXI activity or expression, or at least one symptom associated with the disease or disorder, using an anti-FXI antibody or antigen-binding portion of an antibody of the disclosure. The therapeutic methods according to this aspect of the disclosure comprise administering a therapeutically effective amount of a pharmaceutical composition comprising an antibody or antigen-binding fragment of an antibody of the disclosure to a subject in need thereof. The disorder treated is any disease or condition which is improved, ameliorated, inhibited or prevented by targeting FXI and/or by inactivating FXI-mediated blood clotting.

In one embodiment, the anti-FXI antibodies of the disclosure may provide a method of treating pathological intrinsic clotting, without adversely affecting hemostasis. In one embodiment, the anti-FXI antibodies of the disclosure may provide a method of treating diseases, disorders, clotting side effects, indirect clotting effects of any one of Factor V Leiden, prothrombin gene mutation, deficiencies of natural proteins that prevent clotting (such as antithrombin, protein C and protein S), elevated levels of homocysteine, elevated levels of fibrinogen or dysfunctional fibrinogen (dysfibrinogenemia), elevated levels of factor VIII, factor IX, and/or XI, abnormal fibrinolytic system, including hypoplasminogenemia, dysplasminogenemia and elevation in levels of plasminogen activator inhibitor (PAI-1), atrial fibrillation, cancer, side effects of some medications used to treat cancer, such as tamoxifen, bevacizumab, thalidomide and lenalidomide, recent trauma or surgery, central venous catheter placement, obesity, pregnancy, supplemental estrogen use, including oral contraceptive pills (birth control pills), hormone replacement therapy, prolonged bed rest or immobility, heart attack, congestive heart failure, stroke and other illnesses that lead to decreased activity, heparin-induced thrombocytopenia (decreased platelets in the blood due to heparin or low molecular weight heparin preparations), lengthy airplane travel, antiphospholipid antibody syndrome, deep vein thrombosis or pulmonary embolism, myeloproliferative disorders such as polycythemia vera or essential thrombocytosis, paroxysmal nocturnal hemoglobinuria, inflammatory bowel syndrome, HIV/AIDS, nephrotic syndrome, COVID-19 infection or spike protein immunization effects, and the like.

A seventh aspect of the disclosure provides for a method of preventing thrombosis in a subject without adversely affecting hemostasis, the method comprising administering a therapeutically effective amount of a FXI antagonist antibody of Tables 1A-1C, or a pharmaceutical composition comprising a therapeutically effective amount of the antibody or antigen-binding fragment thereof, to the subject.

In one embodiment, the methods described above may be achieved by administering an antagonist anti-FXI antibody, or antigen-binding fragment thereof, to a subject in need thereof, wherein the antagonist anti-FXI antibody comprises three heavy chain complementarity determining regions (CDRs) (HCDR1, HCDR2 and HCDR3) contained within a heavy chain variable region (HCVR) comprising an amino acid sequence as set forth in Tables 1A-1C, or a substantially similar sequence thereof having at least 90% sequence identity thereto; and three light chain CDRs (LCDR1, LCDR2 and LCDR3) contained within a light chain variable region (LCVR) comprising an amino acid sequence as set forth in Tables 1A-1C, or a substantially similar sequence thereof having at least 90% sequence identity thereto.

In one embodiment, the methods of the disclosure may be achieved by administering an antagonist FXI antibody of the disclosure, wherein the antibody, or antigen-binding fragment thereof, comprises three heavy chain CDRs (HCDR1, HCDR2 and HCDR3) contained within the HC sequence of SEQ ID NO: 18, or a substantially similar sequence thereof having at least 90% sequence identity thereto; and three light chain CDRs (LCDR1, LCDR2 and LCDR3) contained within the LC sequence of SEQ ID NO: 20, or a substantially similar sequence thereof having at least 90% sequence identity thereto.

In one embodiment, the antibody, or antigen-binding fragment thereof, comprises a HC having an amino acid sequence of SEQ ID NO: 18, or an HCVR having an amino acid sequence of an HCVR comprised within SEQ ID NO: 18. In one embodiment, the antibody, or antigen-binding fragment thereof, comprises an HCVR having an amino acid sequence of SEQ ID NO: 2. In one embodiment, the antibody, or antigen-binding fragment thereof, comprises an HCVR having at least 90%, 95%, 96%, 97%, 98%, or 99% identity to an amino acid sequence of SEQ ID NO: 2.

In one embodiment, the antibody, or antigen-binding fragment thereof, comprises a LC having an amino acid sequence of SEQ ID NO: 20, or an LCVR having an amino acid sequence of an LCVR comprised within SEQ ID NO: 20. In one embodiment, the antibody, or antigen-binding fragment thereof, comprises an LCVR having an amino acid sequence of SEQ ID NO: 10. In one embodiment, the antibody, or antigen-binding fragment thereof, comprises an LCVR having an amino acid sequence having at least 90%, 95%, 96%, 97%, 98%, or 99% identity to of SEQ ID NO: 10.

In one embodiment, the antibody, or antigen-binding fragment thereof, comprises a HC having an amino acid sequence of SEQ ID NO: 18; and a LC having an amino acid sequence of SEQ ID NO: 20.

In one embodiment, the antibody, or antigen-binding fragment thereof, comprises the CDRs of a HC/LC amino acid sequence pair of SEQ ID NOs: 18/20.

In one embodiment, the antibody, or antigen-binding fragment thereof, comprises a HCVR/LCVR amino acid sequence pair of SEQ ID NOs: 2/10.

In one embodiment, the antibody, or antigen-binding fragment thereof, comprises:
(a) a HCDR1 domain having an amino acid sequence of SEQ ID NO: 4;
(b) a HCDR2 domain having an amino acid sequence of SEQ ID NO: 6;
(c) a HCDR3 domain having an amino acid sequence of SEQ ID NO: 7;
(d) a LCDR1 domain having an amino acid sequence of SEQ ID NO: 12;
(c) a LCDR2 domain having an amino acid sequence of SEQ ID NO: 14; and
(f) a LCDR3 domain having an amino acid sequence of SEQ ID NO: 16.

In one embodiment, the antibody, or antigen-binding fragment thereof, comprises a set of six CDRs (HCDR1-HCDR2-HCDR3-LCDR1-LCDR2-LCDR3) having amino acid sequences of SEQ ID NOs: 4-6-8-12-14-16.

In one aspect, disclosed herein is a nucleic acid encoding an antibody, or antigen-binding fragment thereof, comprising a set of six CDRs (HCDR1-HCDR2-HCDR3-LCDR1-LCDR2-LCDR3) having amino acid sequences of SEQ ID NOs: 4-6-8-12-14-16. In one embodiment, the nucleic acid encodes an antibody, or an antigen-binding fragment thereof, having an HCVR comprising SEQ ID NO: 2 and/or a LCVR comprising SEQ ID NO: 10. In one embodiment, the nucleic acid encodes an antibody, or an antigen-binding fragment thereof, having an HC comprising SEQ ID NO: 18 and/or a LC comprising SEQ ID NO: 20.

In one aspect, disclosed herein is a nucleic acid encoding an antibody, or antigen-binding fragment thereof, comprising a set of six CDRs (HCDR1-HCDR2-HCDR3-LCDR1-LCDR2-LCDR3) having nucleic acid sequences of SEQ ID NOs: 3-5-7-11-13-15. In one embodiment, the nucleic acid encodes an antibody, or an antigen-binding fragment thereof, having an HCVR comprising SEQ ID NO: 1 and/or a LCVR comprising SEQ ID NO: 9. In one embodiment, the nucleic acid encodes an antibody, or an antigen-binding fragment thereof, having an HC comprising SEQ ID NO: 17 and/or a LC comprising SEQ ID NO: 19.

In one embodiment, the disease or disorder to be treated with an anti-FXI antibody of the disclosure is thrombosis, and any complication resulting from thrombosis.

It is envisioned that any disease or disorder associated with FXI activity or expression is amenable to treatment with an antibody of the disclosure. These disorders may include any disease or condition in which harmful clot formation is a risk, especially, but not limited to, those conditions in which intrinsic clotting and hemostasis are a risk to the patient.

Other embodiments will become apparent from a review of the ensuing detailed description.

### BRIEF DESCRIPTION OF THE FIGURES

FIG. 1 depicts a size analysis of complexes formed between human coagulation factor XI and REGN7528 and REGN7531.

FIG. 2 depicts a size analysis of complexes formed between human coagulation factor XI and REGN7503, REGN7505, and REGN7508.

FIG. 3 depicts a size analysis of complexes formed between human coagulation factor XI and REGN7508.

FIG. 4 depicts a size analysis of complexes formed between human coagulation factor XI and REGN9932.

FIG. 5 depicts a comparison of size analyses of complexes formed between human coagulation factor XI and REGN7508 with complexes formed between human coagulation factor XI and REGN9932.

FIGS. 6A-6C show a comparison of the effects of anti-FXI mAbs on the intrinsic coagulation pathway in female cynomolgus monkey plasma using thrombin generation assays (TGAs).

FIGS. 7A-7C show a comparison of the effects of anti-FXI mAbs on the extrinsic coagulation pathway in female cynomolgus monkey plasma using TGAs.

FIGS. 8A-8C show a comparison of the effects of anti-FXI mAbs on the intrinsic coagulation pathway in pooled female human plasma using TGAs.

FIGS. 9A-9C show a comparison of the effects of anti-FXI mAbs on the intrinsic coagulation pathway in pooled female human plasma using TGAs.

FIGS. 10A-10C show the effects of REGN9932 on the intrinsic coagulation pathway in six single human donors using TGAs.

FIGS. 11A-11C show the effects of REGN7508 on the intrinsic coagulation pathway in six single human donors using TGAs.

FIGS. 12A-12C show the effects of REGN9932 on the extrinsic coagulation pathway in six single human donors using TGAs.

FIGS. 13A-13C show the effects of REGN7508 on the extrinsic coagulation pathway in six single human donors using TGAs.

FIG. 14 is an overview of the coagulation pathway.

FIG. 15 is an example of a thrombogram.

FIGS. 16A-16C show the effect of REGN7508 on the intrinsic and extrinsic coagulation pathways in pooled human donor plasma using clotting assays.

FIGS. 17A-17C show the effect of REGN7508 on the intrinsic and extrinsic coagulation pathways in pooled cynomolgus monkey donor plasma using clotting assays.

FIGS. 18A-18D show the effect of REGN7508 on the intrinsic and extrinsic coagulation pathways in pooled human donor plasma using TGAs.

FIG. 19 shows the effect of REGN7508 at a second set of concentrations on the intrinsic coagulation pathways in pooled human donor plasma using TGAs.

FIGS. 20A-20D show the effect of REGN7508 on the intrinsic and extrinsic coagulation pathways in pooled cynomolgus monkey donor plasma using TGAs.

FIG. 21 shows the effect of REGN7508 at a second set of concentrations on the intrinsic coagulation pathways in pooled cynomolgus monkey donor plasma using TGAs.

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present disclosure, the preferred methods and materials are now described. All patents, applications and non-patent publications mentioned in this specification are incorporated herein by reference in their entireties.

### Definitions

The expression “FXI,” and the like, also known as “coagulation factor XI” or “Factor XI”, refers to the human plasma serine protease (unless designated as being from another species) comprising the amino acid sequence as set forth in amino acid residues 19 through 625 of accession number NP_000119.1 (SEQ ID NO: 41). Human FXI containing a myc-myc-hexahistidine tag is shown as SEQ ID NO: 42 (with amino acid residues 1-607 being human FXI and amino acid residues 608-635 being the myc-myc-hexahistidine tag).

In certain instances, cell lines were prepared that expressed the FXI proteins, subunits of the FXI protein, and chimera proteins containing one or more FXI subunits, tag sequences, and plasma kallikrein protein sequences. For example, SEQ ID NO: 43 (construct hFXI_PKA1) is a chimera containing, at amino acids 1-85, the apple 1 domain (PKA1) of human kallikrein B1 (amino acids G20-C104 of human kallilrein B1 [SEQ ID NO: 48]), at amino acids 86-60, amino acids H105-V625 of human FXI (hFXI), and at amino acids 607-634, the myc-myc-hexagistidine tag.

For example, SEQ ID NO: 44 (construct hFXI_PKA2) is a chimera containing, at amino acids 1-90, amino acids E19-S108 of hFXI, at amino acids 91-174, the apple 2 domain (PKA2, also referred to as “A2”) of hKLKB1 (amino acids C111-C193 SEQ ID NO: 48), at amino acids 175-605, amino acids A195-V625 of hFXI, and at amino acids 606-633, the myc-myc-hexagistidine tag.

For example, SEQ ID NO: 45 (construct hFXI_PKA3) is a chimera containing, at amino acids 1-180, amino acids E19-L198 of hFXI, at amino acids 181-264, the apple 3 domain (PKA3) of hKLKB1 (amino acids C201-C284 SEQ ID NO: 48), at amino acids 265-605, amino acids H285-V625 of hFXI, and at amino acids 606-633, the myc-myc-hexagistidine tag.

For example, SEQ ID NO: 46 (construct hFXI_PKA4) is a chimera containing, at amino acids 1-271, amino acids E19-V289 of hFXI, at amino acids 272-355, the apple 4 domain (PKA4) of hKLKB1 (amino acids C292-C375 SEQ ID NO: 48), at amino acids 356-605, amino acids M376-V625 of hFXI, and at amino acids 606-633, the myc-myc-hexagistidine tag.

For example, SEQ ID NO: 47 (construct hKLKB1.mmh) is a chimera containing, at amino acids 1-619, amino acids G20-A638 of hKLKB1, and at amino acids 620-647, the myc-myc-hexagistidine tag.

As used herein, the expression “anti-FXI antibody” includes both monovalent antibodies with a single specificity, as well as bispecific antibodies comprising a first arm that binds FXI and a second arm that binds a second (target) antigen, wherein the anti-FXI arm comprises any of the HCVR/LCVR or CDR sequences as set forth in Tables 1A-1C herein. The expression “anti-FXI antibody” also includes antibody-drug conjugates (ADCs) comprising an anti-FXI antibody or antigen-binding portion thereof conjugated to a drug or toxin (i.e., cytotoxic agent). The expression “anti-FXI antibody” also includes antibody-radionuclide conjugates (ARCs) comprising an anti-FXI antibody or antigen-binding portion thereof conjugated to a radionuclide.

The term “anti-FXI antibody”, as used herein, means any antigen-binding molecule or molecular complex comprising at least one complementarity determining region (CDR) that specifically binds to or interacts with FXI or a portion of FXI or a catalytic domain of FXI or an epitope within a catalytic domain of FXI. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or Vu) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-FXI antibody (or antigen-binding portion thereof) may be identical to the human germline sequences, or may be naturally or artificially modified. An amino acid consensus sequence may be defined based on a side-by-side analysis of two or more CDRs.

The term “antibody”, as used herein, also includes antigen-binding fragments of full length antibody molecules. The terms “antigen-binding portion” of an antibody, “antigen-binding fragment” of an antibody, and the like, as used herein, include any enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present disclosure using routine techniques available in the art.

In certain instances, it may be desirable to antagonize FXI, for example, for inhibiting the formation of blood clots. However, the antibodies of the present disclosure act as antagonist antibodies, which serve as inhibitors of FXI or FXIa activity and concomitantly serve as inhibitors of intrinsic pathway thrombosis/clot formation. The antibodies of the present disclosure may function by preventing the interaction between FXI and its upstream activators coagulation factor XII (FXII) and/or coagulation faction II (FII or thrombin). The antibodies of the present disclosure may also function by preventing the interaction between FXI and its downstream target coagulation factor IX (FIX). The antibodies of the present disclosure may also function by sequestering FXI from the blood stream of a patient.

The term “human antibody”, as used herein, is intended to include non-naturally occurring human antibodies. The term includes antibodies that are recombinantly produced in a non-human mammal, or in cells of a non-human mammal. The term is not intended to include antibodies isolated from or generated in a human subject.

The antibodies of the disclosure may, in some embodiments, be recombinant and/or non-naturally occurring human antibodies. The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. In certain embodiments, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant disclosure encompasses antibodies having one or more mutations in the hinge, C_{H}2 or C_{H}3 region, which may be desirable, for example, in production, to improve the yield of the desired antibody form.

The term “specifically binds”, or “binds specifically to”, or the like, means that an antibody, or antigen-binding fragment thereof, forms a complex with an antigen that is relatively stable under physiologic conditions. Specific binding can be characterized by an equilibrium dissociation constant of at least about 1×10⁻⁶ M or less (e.g., a smaller K_{D} denotes a tighter binding). Methods for determining whether two molecules specifically bind are well known in the art and include, for example, equilibrium dialysis, surface plasmon resonance, and the like. As described herein, antibodies have been identified by surface plasmon resonance, e.g., BIACORE™, which bind specifically to FXI. Moreover, multi-specific antibodies that bind to FXI protein and one or more additional antigens or a bi-specific that binds to two different regions of FXI are nonetheless considered antibodies that “specifically bind”, as used herein.

The antibodies of the disclosure may be isolated antibodies. An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present disclosure. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The anti-FXI antibodies disclosed herein may comprise one or more amino acid substitutions, insertions and/or deletions in the framework and/or CDR regions of the heavy and light chain variable domains. Such mutations can be readily ascertained by comparing the amino acid sequences disclosed herein to sequences available from, for example, public antibody sequence databases. Once obtained, antibodies and antigen-binding fragments that contain one or more mutations can be easily tested for one or more desired property such as, improved binding specificity, increased binding affinity, improved or enhanced antagonistic or antagonistic biological properties (as the case may be), reduced immunogenicity, etc. Antibodies and antigen-binding fragments obtained in this general manner are encompassed within the present disclosure.

The present disclosure also includes anti-FXI antibodies comprising variants of any of the HCVR, LCVR, and/or CDR amino acid sequences disclosed herein having one or more conservative substitutions. For example, the present disclosure includes anti-FXI antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences set forth in Tables 1A-1C herein.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains; phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the disclosure to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

### Characteristics of the Antibodies

The present disclosure includes anti-FXI antibodies that bind the catalytic domain of human FXI with a K_{D} of less than about 500 pM as measured by surface plasmon resonance at 25° C., or at 37° C. According to certain embodiments, the disclosure includes anti-FXI antibodies that bind human FXI with a K_{D} of less than about 400 pM, less than about 300 PM, less than about 200 pM, less than about 150 pM, less than about 100 pM, less than about 80 pM, less than about 50 pM, less than about 40 pM, less than about 30 pM, less than about 20 pM, less than about 10 pM, less than about 5 pM, less than about 3 pM, or less than about 1 pM.

The present disclosure includes anti-FXI antibodies that bind activated human FXI (FXIa) with a K_{D} of less than about 1,000 pM as measured by surface plasmon resonance at 25° C., or at 37° C. According to certain embodiments, the disclosure includes anti-FXI antibodies that bind human FXI with a K_{D} of less than about 900 pM, less than about 800 pM, less than about 700 pM, less than about 500 pM, less than about 250 pM, less than about 100 pM, less than about 80 pM, less than about 50 pM, less than about 40 pM, less than about 30 pM, less than about 20 pM, less than about 10 pM, less than about 5 pM, less than about 3 pM, or less than about 1 pM.

The present disclosure includes anti-FXI antibodies that bind human FXI with a dissociative half life (t½) of greater than about 10 minutes as measured by surface plasmon resonance at 25° C., or 37° C. According to certain embodiments, the disclosure includes anti-FXI antibodies that bind human FXI with a t½ of greater than about 20 minutes, greater than about 50 minutes, greater than about 100 minutes, greater than about 120 minutes, greater than about 150 minutes, greater than about 300 minutes, greater than about 350 minutes, greater than about 400 minutes, greater than about 450 minutes, greater than about 500 minutes, greater than about 550 minutes, greater than about 600 minutes, greater than about 700 minutes, greater than about 800 minutes, greater than about 900 minutes, greater than about 1000 minutes, greater than about 1100 minutes, or greater than about 1200 minutes.

The present disclosure includes anti-FXI antibodies that bind human FXIa with a dissociative half life (t½) of greater than about 10 minutes as measured by surface plasmon resonance at 25° C., or 37° C. According to certain embodiments, the disclosure includes anti-FXI antibodies that bind human FXIa with a t½ of greater than about 20 minutes, greater than about 50 minutes, greater than about 100 minutes, greater than about 120 minutes, greater than about 150 minutes, greater than about 300 minutes, greater than about 350 minutes, greater than about 400 minutes, greater than about 450 minutes, greater than about 500 minutes, greater than about 550 minutes, greater than about 600 minutes, greater than about 700 minutes, greater than about 800 minutes, greater than about 900 minutes, greater than about 1000 minutes, greater than about 1100 minutes, or greater than about 1200 minutes.

The present disclosure includes anti-FXI antibodies that may or may not bind non-human FXI. As used herein, an antibody “does not bind” a particular antigen (e.g., monkey, mouse or rat FXI if the antibody, when tested in an antigen binding assay such as surface plasmon resonance exhibits a K_{D} of greater than about 1000 nM, or does not exhibit any antigen binding, in such an assay. Another assay format that can be used to determine whether an antibody binds or does not bind a particular antigen, according to this aspect of the disclosure, is ELISA.

It is generally known in the art that activated FXI (FXIa) activates Factor IX by selectively cleaving arg-ala and arg-val peptide bonds. Factor IXa, in turn, forms a complex with Factor VIIIa (FIXa-FVIIIa) and activates Factor X. The present disclosure includes anti-FXI antibodies that inhibit FXI-mediated activation of human FX in plasma by at least about 85% with an IC₅₀ of less than about 100 pM. Using an assay format described in Example 4, or a substantially similar assay format, an IC₅₀ value can be calculated as the concentration of antibody required to activate FXI-mediated signaling to the half-maximal signal observed. Thus, according to certain embodiments, the disclosure includes anti-FXI antibodies that mediate human FXI-mediated activation of human FX in plasma by at least about 85% with an IC₅₀ of less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, or less than about 5 pM, as measured using the assay format described in Example 4 herein or a substantially similar assay.

The present disclosure also includes anti-FXI antibodies that inhibit FXIa-mediated activation of human FX in plasma by at least about 25% with an IC₅₀ of less than about 50 pM. Using an assay format described in Example 4, or a substantially similar assay format, an IC₅₀ value can be calculated as the concentration of antibody required to activate FXIa-mediated signaling to the half-maximal signal observed. Thus, according to certain embodiments, the disclosure includes anti-FXI antibodies that mediate human FXIa-mediated activation of human FX in plasma by at least about 25% with an IC₅₀ of less than about 200 pM, less than about 300 pM, less than about 200 pM, less than about 100 pM, less than about 50 pM, less than about 40 pM, less than about 30 pM, less than about 20 pM, less than about 10 pM, less than about 9 pM, less than about 8 pM, less than about 7 pM, less than about 6 pM, less than about 5 pM, less than about 4 pM, less than about 3 pM, less than about 2 pM, or less than about 1 pM, as measured using the assay format described in Example 4 herein or a substantially similar assay.

The present disclosure includes anti-FXI antibodies that preferentially bind to the catalytic domain (CAT) as demonstrated by direct binding to CAT domain constructs or by competing with one or more specific CAT-binding antibodies as shown in Example 5 and Example 6 respectively. In one embodiment, the antibodies, or antigen-binding fragments thereof, disclosed herein do not bind an apple domain (e.g., an A2 domain) of FXI.

The present disclosure includes anti-FXI antibodies that prolong the activated partial thromboplastin time (aPTT), which is a measure of intrinsic pathway thrombogenesis, while having no measureable effect on prothrombin time (PT), which is a measure of extrinsic pathway thrombogenesis, in human plasma. In one embodiment, the aPTT is measured in pooled human plasma treated with ellagic acid and the PT is measured in pooled human plasma treated with tissue factor using a hemostasis analyzer as exemplified in Example 6 and Example 9. It is generally known in the art that ellagic acid stimulates the intrinsic pathway of thrombogenesis in vitro and tissue factor stimulates the extrinsic pathway of thrombogenesis. Here, the anti-FXI antibody prolongs aPTT about two-fold at a concentration of less than 100 nM, less than 90 nM, less than 80 nM, less than 70 nM, less than 60 nM, less than 50 nM, less than 40 nM, less than 30 nM, less than 20 nM, less than 10 nM, 1 nM-100 nM, 1 nM-100 nM, 1 nM-50 nM, 100 pM-50 nM, 5 nM-50 nM, 5 nM-40 nM, 5 nM-15 nM, 10 nM-20 nM, 15 nM-25 nM, 20 nM-30 nM, 25 nM-35 nM, 30 nM-40 nM, 35 nM-45 nM, 40 nM-50 nM, 45 nM-55 nM, 50 nM-60 nM, 55 nM-65 nM, 60 nM-100 nM, 65 nM-75 nM, 70 nM-80 nM, 75 nM-85 nM, 80 nM-90 nM, 85 nM-95 nM, 90 nM-100 nM, or 95 nM-105 nM, without doubling PT.

The present disclosure includes anti-FXI antibodies that inhibit the production of thrombin via the intrinsic pathway (intrinsic thrombin) in human plasma in vitro with little to no effect on the production of thrombin via the extrinsic pathway (extrinsic thrombin). In one embodiment, pathway-specific thrombin production is determined in vitro by a thrombin generation assay using a calibrated automated thrombogram as exemplified in Example 6 and Example 9. Here, a thrombin generation profile is generated and peak thrombin concentration is determined in ellagic acid treated plasma and in tissue factor treated plasma with and without an anti-FXI antibody. Thus, in one embodiment, anti-FXI antibodies inhibit the production of intrinsic thrombin at a concentration of 0.1 nM-100 nM, 1 nM-100 nM, 5 nM-500 nM, 5 nM-100 nM, 10 nM-100 nM, 10 nM-50 nM, 5 nM-15 nM, 10 nM-20 nM, 25 nM-35 nM, 30 nM-40 nM, 35 nM-45 nM, 40 nM-50 nM, 45 nM-55 nM, 50 nM-60 nM, 55 nM-65 nM, 60 nM-65 nM, ≥20 nM, ≥25 nM, ≥30 nM, ≥35 nM, ≥40 nM, ≥45 nM, ≥50 nM, ≥55 nM, ≥5 nM, ≥10 nM, or ≥15 nM. Here, the anti-FXI antibodies have no effect on the production of extrinsic thrombin with any concentration up to 500 nM.

The present disclosure includes anti-FXI antibodies that increase by at least two-fold activated partial thromboplastin time (aPTT) in a primate in vivo without measurably affecting prothrombin time (PT). Here, a primate is administered an anti-FXI antibody, plasma is obtained from the primate, the plasma is contacted with ellagic acid or tissue factor, and then the aPTT or PT respectively is determined in an assay as exemplified in Example 7. In one embodiment, the primate is a human. In one embodiment, the primate is a monkey.

In one embodiment, the anti-FXI antibody is administered at a dose of 0.01 mg/kg-20 mg/kg, 0.1 mg/kg-10 mg/kg, 1 mg/kg-10 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, or about 15 mg/kg.

In one embodiment, the aPTT is prolonged with the anti-FXI treatment relative to no anti-FXI treatment at least 1.5-fold, at least 2-fold, at least 2.5-fold, at least 3-fold, at least 3.5-fold, at least 4-fold, at least 4.5-fold, at least 5-fold, at least 5.5-fold, or at least 6-fold.

In one embodiment, the anti-FXI-mediated aPTT prolongation effect persists in the subject after receiving a dose of the anti-FXI antibody for at least 1 week, at least 2 weeks, at least 3 weeks, at least 4 weeks, at least 5 weeks, at least 6 weeks, at least 7 weeks, at least 8 weeks, at least 3 months, at least 4 months, at least 5 months, or at least 6 months.

The present disclosure includes anti-FXI antibodies that inhibit intrinsic pathway peak thrombin activity in a primate in vivo without measurably affecting extrinsic pathway peak thrombin activity. Here, a primate is administered an anti-FXI antibody, plasma is obtained from the primate, the plasma is contacted with ellagic acid or tissue factor, and then the generation of intrinsic thrombin or extrinsic thrombin respectively is determined in a thrombin generation assay as exemplified in Example 7. In one embodiment, the primate is a human. In one embodiment, the primate is a monkey.

In one embodiment, the anti-FXI antibody is administered at a dose of 0.01 mg/kg-20 mg/kg, 0.1 mg/kg-10 mg/kg, 1 mg/kg-10 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, or about 15 mg/kg.

In one embodiment, the peak intrinsic thrombin (i.e., the thrombin generated via ellagic acid) activity in the anti-FXI treatment relative to no anti-FXI treatment is inhibited by 1%-100%, 5%-95%, 10%-90%, 20%-80%, 1%-10%, 5%-20%, 10%-30%, 15%-40%, 20%-50%, 25%-60%, 5%-15%, 10%-20%, 15%-25%, 20%-30%, 25%-35%, 30%-40%, 35%-45%, 40%-50%, 45%-55%, 50%-60%, 55%-65%, 60%-70%, 65%-75%, 70%-80%, 75%-85%, 80%-90%, 85%-95%, 90%-100%, 95%-105%, or ≥100%.

In one embodiment, the anti-FXI-mediated inhibition of peak intrinsic thrombin activity persists in the subject after receiving a dose of the anti-FXI antibody for at least 1 week, at least 2 weeks, at least 3 weeks, at least 4 weeks, at least 5 weeks, at least 6 weeks, at least 7 weeks, at least 8 weeks, at least 3 months, at least 4 months, at least 5 months, or at least 6 months.

A binding characteristic of an antibody of the disclosure (e.g., any of the binding characteristics mentioned herein above), when disclosed in term of being “measured by surface plasmon resonance” means that the relevant binding characteristic pertaining to the interaction between the antibody and the antigen are measured using a surface plasmon resonance instrument (e.g., a Biacore® instrument, GE Healthcare) using standard Biacore assay conditions as illustrated in Example 3 herein, or substantially similar assay format. In certain embodiments, the binding parameters are measured at 25° C., while in other embodiments, the binding parameters are measured at 37° C.

The present disclosure includes antibodies or antigen-binding fragments thereof that specifically bind FXI, comprising an HCVR and/or an LCVR comprising an amino acid sequence selected from any of the HCVR and/or LCVR amino acid sequences listed in Tables 1A-1C.

The antibodies of the present disclosure may possess one or more of the aforementioned biological characteristics, or any combination thereof. The foregoing list of biological characteristics of the antibodies of the disclosure is not intended to be exhaustive. Other biological characteristics of the antibodies of the present disclosure will be evident to a person of ordinary skill in the art from a review of the present disclosure including the working Examples herein.

### Epitope Mapping and Related Technologies

The epitope to which the antibodies of the present disclosure bind may consist of a single contiguous sequence of 3 or more (e.g., 3, 4, 5, 6, 7, 8, 9, 10, 11, 12, 13, 14, 15, 16, 17, 18, 19, 20 or more) amino acids of a FXI protein. Alternatively, the epitope may consist of a plurality of non-contiguous amino acids (or amino acid sequences) of FXI. In some embodiments, the epitope is located on or near a surface of FXI, for example, in the domain that interacts with any one of its ligands, e.g., FXIIa, thrombin, and FIX. In other embodiments, the epitope is located on or near a surface of FXI that does not interact with the FXI ligand, e.g., at a location on the surface of FXI at which an antibody, when bound to such an epitope, does not interfere with the interaction between FXI and its ligand.

Various techniques known to persons of ordinary skill in the art can be used to determine whether an antibody “interacts with one or more amino acids” within a polypeptide or protein. Exemplary techniques include, e.g., routine cross-blocking assay such as that described Antibodies, Harlow and Lane (Cold Spring Harbor Press, Cold Spring Harb., NY), alanine scanning mutational analysis, peptide blots analysis (Reinecke, 2004, Methods Mol Biol 248:443-463), and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267(2):252-259; Engen and Smith (2001) Anal. Chem. 73:256A-265A.

The present disclosure includes anti-FXI antibodies that bind to the same epitope as any of the specific exemplary antibodies described herein (e.g. antibodies comprising any of the amino acid sequences as set forth in Tables 1A-1C herein). Likewise, the present disclosure also includes anti-FXI antibodies that compete for binding to FXI with any of the specific exemplary antibodies described herein (e.g. antibodies comprising any of the amino acid sequences as set forth in Tables 1A-1C herein).

One can easily determine whether an antibody binds to the same epitope as, or competes for binding with, a reference anti-FXI antibody by using routine methods known in the art and exemplified herein. For example, to determine if a test antibody binds to the same epitope as a reference anti-FXI antibody of the disclosure, the reference antibody is allowed to bind to an FXI protein. Next, the ability of a test antibody to bind to the FXI molecule is assessed. If the test antibody is able to bind to FXI following saturation binding with the reference anti-FXI antibody, it can be concluded that the test antibody binds to a different epitope than the reference anti-FXI antibody. On the other hand, if the test antibody is not able to bind to the FXI molecule following saturation binding with the reference anti-FXI antibody, then the test antibody may bind to the same epitope as the epitope bound by the reference anti-FXI antibody of the disclosure. Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present disclosure, two antibodies bind to the same (or overlapping) epitope if, e.g., a 1-, 5-, 10-, 20- or 100-fold excess of one antibody inhibits binding of the other by at least 50% but preferably 75%, 90% or even 99% as measured in a competitive binding assay (sec, e.g., Junghans et al., Cancer Res. 1990:50:1495-1502). Alternatively, two antibodies are deemed to bind to the same epitope if essentially all amino acid mutations in the antigen that reduce or eliminate binding of one antibody reduce or eliminate binding of the other. Two antibodies are deemed to have “overlapping epitopes” if only a subset of the amino acid mutations that reduce or eliminate binding of one antibody reduce or eliminate binding of the other.

To determine if an antibody competes for binding (or cross-competes for binding) with a reference anti-FXI antibody, the above-described binding methodology is performed in two orientations: In a first orientation, the reference antibody is allowed to bind to an FXI protein under saturating conditions followed by assessment of binding of the test antibody to the FXI molecule. In a second orientation, the test antibody is allowed to bind to a FXI molecule under saturating conditions followed by assessment of binding of the reference antibody to the FXI molecule. If, in both orientations, only the first (saturating) antibody is capable of binding to the FXI molecule, then it is concluded that the test antibody and the reference antibody compete for binding to FXI (see, e.g., the assay format described in the Examples herein, in which FXI protein is captured onto sensor tips and the FXI-coated sensor tips are treated with a reference antibody and a test anti-FXI antibody sequentially and in both binding orders). As will be appreciated by a person of ordinary skill in the art, an antibody that competes for binding with a reference antibody may not necessarily bind to the same epitope as the reference antibody, but may sterically block binding of the reference antibody by binding an overlapping or adjacent epitope.

### Preparation of Human Antibodies

The anti-FXI antibodies of the present disclosure can be fully human but non-naturally occurring, antibodies. Methods for generating monoclonal antibodies, including fully human monoclonal antibodies are known in the art. Any such known methods can be used in the context of the present disclosure to make human antibodies that specifically bind to human FXI.

Using VELOCIMMUNE® technology (see, for example, U.S. Pat. No. 6,596,541, Regeneron Pharmaceuticals, VELOCIMMUNE®) or any other known method for generating monoclonal antibodies, high affinity chimeric antibodies to an allergen are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

As described in the experimental section below, the high affinity chimeric antibodies, which are isolated having a human variable region and a mouse constant region, are characterized and selected for desirable characteristics, including affinity, selectivity, epitope, etc. The mouse constant regions are then replaced with a desired human constant region to generate the fully human antibody of the disclosure, for example wild-type or modified IgG1 or IgG4. While the constant region selected may vary according to specific use, high affinity antigen-binding and target specificity characteristics reside in the variable region.

In certain embodiments, it may be desirable to test anti-human FXI antibodies in mice or rats that have been engineered to express a human FXI receptor. These mice or rats may be beneficial in circumstances wherein the anti-FXI antibodies may only bind human FXI, but will not cross react with mouse or rat FXI. Any method known to those skilled in the art may be used for generating such FXI humanized mice and rats.

In general, the antibodies of the instant disclosure possess very high affinities, typically possessing K_{D} of from about 10⁻¹² through about 10⁻⁹ M, when measured by binding to antigen either immobilized on solid phase or in solution phase.

### Bioequivalents

The anti-FXI antibodies and antibody fragments of the present disclosure encompass proteins having amino acid sequences that vary from those of the described antibodies but that retain the ability to bind human FXI. Such variant antibodies and antibody fragments comprise one or more additions, deletions, or substitutions of amino acids when compared to parent sequence, but exhibit biological activity that is essentially equivalent to that of the described antibodies. Likewise, the anti-FXI antibody-encoding DNA sequences of the present disclosure encompass sequences that comprise one or more additions, deletions, or substitutions of nucleotides when compared to the disclosed sequence, but that encode an anti-FXI antibody or antibody fragment that is essentially bioequivalent to an anti-FXI antibody or antibody fragment of the disclosure. Examples of such variant amino acid and DNA sequences are discussed above.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of anti-FXI antibodies of the disclosure may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include anti-FXI antibody variants comprising amino acid changes which modify the glycosylation characteristics of the antibodies, e.g., mutations which eliminate or remove glycosylation.

### Species Selectivity and Species Cross-Reactivity

The present disclosure, according to certain embodiments, provides anti-FXI antibodies that bind to human FXI but not to FXI from other species. The present disclosure also includes anti-FXI antibodies that bind to human FXI and to FXI from one or more non-human species. For example, the anti-FXI antibodies of the disclosure may bind to human FXI and may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee FXI. According to certain exemplary embodiments of the present disclosure, anti-FXI antibodies are provided which specifically bind human FXI but do not bind, or bind only weakly, to mouse or rat FXI.

### Multispecific Antibodies

The antibodies of the present disclosure may be monospecific or multispecific (e.g., bispecific). Multispecific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. The anti-FXI antibodies of the present disclosure can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second binding specificity.

The present disclosure includes bispecific antibodies wherein one arm of an immunoglobulin binds human FXI, and the other arm of the immunoglobulin is specific for a second antigen. The FXI-binding arm can comprise any of the HCVR/LCVR or CDR amino acid sequences as set forth in Tables 1A-1C herein.

An exemplary bispecific antibody format that can be used in the context of the present disclosure involves the use of a first immunoglobulin (Ig) C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies. Variations on the bispecific antibody format described above are contemplated within the scope of the present disclosure.

Other exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

### Therapeutic Formulations and Administration

The disclosure provides pharmaceutical compositions comprising the anti-FXI antibodies or antigen-binding fragments thereof of the present disclosure. The pharmaceutical compositions of the disclosure are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antibody administered to a patient may vary depending upon the age and the size of the patient, target disease, conditions, route of administration, and the like. The preferred dose is typically calculated according to body weight or body surface area. In an adult patient, it may be advantageous to intravenously administer the antibody of the present disclosure normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. Effective dosages and schedules for administering anti-FXI antibodies may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intravitreal, intraocular, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park IL), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, intravitreal, intraocular, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

### Therapeutic Uses of the Antibodies

The present disclosure includes methods comprising administering to a subject in need thereof a therapeutic composition comprising an anti-FXI antibody (e.g., an anti-FXI antibody comprising any of the HCVR/LCVR or CDR sequences as set forth in Tables 1A-1C herein). The therapeutic composition can comprise any one or more of the anti-FXI antibodies or antigen-binding fragments thereof disclosed herein, and a pharmaceutically acceptable carrier or diluent.

The antibodies of the disclosure are useful, inter alia, for the treatment, prevention and/or amelioration of any disease or disorder associated with or mediated by FXI expression or activity. The FXI antagonist antibodies of the disclosure may be used to treat or prevent thrombosis, especially thrombosis of the intrinsic pathway, while minimizing negatively impacting hemostasis and clot formation via the extrinsic pathway.

The present disclosure includes methods of treating or preventing thrombosis by administering to a patient in need of such treatment an anti-FXI antibody, or antigen-binding fragment thereof, as disclosed elsewhere herein.

In one embodiment, the anti-FXI antibodies of the disclosure may provide a method of treating or preventing thrombosis associated with any one or more of Factor V Leiden, prothrombin gene mutation, deficiencies of natural proteins that prevent clotting (such as antithrombin, protein C and protein S), elevated levels of homocysteine, elevated levels of fibrinogen or dysfunctional fibrinogen (dysfibrinogenemia), elevated levels of factor VIII, factor IX, and/or XI, abnormal fibrinolytic system, including hypoplasminogenemia, dysplasminogenemia and elevation in levels of plasminogen activator inhibitor (PAI-1), atrial fibrillation, cancer, side effects of some medications used to treat cancer, such as tamoxifen, bevacizumab, thalidomide and lenalidomide, recent trauma or surgery, central venous catheter placement, obesity, pregnancy, supplemental estrogen use, including oral contraceptive pills (birth control pills), hormone replacement therapy, prolonged bed rest or immobility, heart attack, congestive heart failure, stroke and other illnesses that lead to decreased activity, heparin-induced thrombocytopenia (decreased platelets in the blood due to heparin or low molecular weight heparin preparations), lengthy airplane travel, antiphospholipid antibody syndrome, deep vein thrombosis or pulmonary embolism, myeloproliferative disorders such as polycythemia vera or essential thrombocytosis, paroxysmal nocturnal hemoglobinuria, inflammatory bowel syndrome, HIV/AIDS, nephrotic syndrome, COVID-19 infection or spike protein immunization effects, and the like.

In the context of the methods of treatment described herein, the anti-FXI antibody may be administered as a monotherapy (i.e., as the only therapeutic agent) or in combination with one or more additional therapeutic agents.

### Combination Therapies and Formulations

The present disclosure includes compositions and therapeutic formulations comprising any of the anti-FXI antibodies described herein in combination with one or more additional therapeutically active components, and methods of treatment comprising administering such combinations to subjects in need thereof.

The anti-FXI antibodies of the present disclosure may be co-formulated with one or more drugs used to treat Factor V Leiden, prothrombin gene mutation, deficiencies of natural proteins that prevent clotting (such as antithrombin, protein C and protein S), elevated levels of homocysteine, elevated levels of fibrinogen or dysfunctional fibrinogen (dysfibrinogenemia), elevated levels of factor VIII, factor IX, and/or XI, abnormal fibrinolytic system, including hypoplasminogenemia, dysplasminogenemia and elevation in levels of plasminogen activator inhibitor (PAI-1), atrial fibrillation, cancer, side effects of some medications used to treat cancer, such as tamoxifen, bevacizumab, thalidomide and lenalidomide, recent trauma or surgery, central venous catheter placement, obesity, pregnancy, supplemental estrogen use, including oral contraceptive pills (birth control pills), hormone replacement therapy, prolonged bed rest or immobility, heart attack, congestive heart failure, stroke and other illnesses that lead to decreased activity, heparin-induced thrombocytopenia (decreased platelets in the blood due to heparin or low molecular weight heparin preparations), lengthy airplane travel, antiphospholipid antibody syndrome, deep vein thrombosis or pulmonary embolism, myeloproliferative disorders such as polycythemia vera or essential thrombocytosis, paroxysmal nocturnal hemoglobinuria, inflammatory bowel syndrome, HIV/AIDS, nephrotic syndrome, COVID-19 infection or spike protein immunization effects, and the like.

The anti-FXI antibodies of the disclosure may also be administered and/or co-formulated in combination with antivirals, antibiotics, analgesics, antioxidants, COX inhibitors, and/or NSAIDs. The anti-FXI antibodies may also be used in conjunction with other types of therapy including stem cell therapy, glaucoma filtration surgery, laser surgery, or gene therapy.

The additional therapeutically active component(s), e.g., any of the agents listed above or derivatives thereof, may be administered just prior to, concurrent with, or shortly after the administration of an anti-FXI antibody of the present disclosure; (for purposes of the present disclosure, such administration regimens are considered the administration of an anti-FXI antibody “in combination with” an additional therapeutically active component). The present disclosure includes pharmaceutical compositions in which an anti-FXI antibody of the present disclosure is co-formulated with one or more of the additional therapeutically active component(s) as described elsewhere herein.

### Administration Regimens

According to certain embodiments of the present disclosure, multiple doses of an anti-FXI antibody (or a pharmaceutical composition comprising a combination of an anti-FXI antibody and any of the additional therapeutically active agents mentioned herein) may be administered to a subject over a defined time course. The methods according to this aspect of the disclosure comprise sequentially administering to a subject multiple doses of an anti-FXI antibody of the disclosure. As used herein, “sequentially administering” means that each dose of anti-FXI antibody is administered to the subject at a different point in time, e.g., on different days separated by a predetermined interval (e.g., hours, days, weeks or months). The present disclosure includes methods which comprise sequentially administering to the patient a single initial dose of an anti-FXI antibody, followed by one or more secondary doses of the anti-FXI antibody, and optionally followed by one or more tertiary doses of the anti-FXI antibody.

The terms “initial dose,” “secondary doses,” and “tertiary doses,” refer to the temporal sequence of administration of the anti-FXI antibody of the disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of anti-FXI antibody, but generally may differ from one another in terms of frequency of administration. In certain embodiments, however, the amount of anti-FXI antibody contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of the present disclosure, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of anti-FXI antibody, which is administered to a patient prior to the administration of the very next dose in the sequence with no intervening doses.

The methods according to this aspect of the disclosure may comprise administering to a patient any number of secondary and/or tertiary doses of an anti-FXI antibody. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient. The administration regimen may be carried out indefinitely over the lifetime of a particular subject, or until such treatment is no longer therapeutically needed or advantageous.

In embodiments involving multiple secondary doses, each secondary dose may be administered at the same frequency as the other secondary doses. For example, each secondary dose may be administered to the patient 1 to 2 weeks or 1 to 2 months after the immediately preceding dose. Similarly, in embodiments involving multiple tertiary doses, each tertiary dose may be administered at the same frequency as the other tertiary doses. For example, each tertiary dose may be administered to the patient 2 to 12 weeks after the immediately preceding dose. In certain embodiments of the disclosure, the frequency at which the secondary and/or tertiary doses are administered to a patient can vary over the course of the treatment regimen. The frequency of administration may also be adjusted during the course of treatment by a physician depending on the needs of the individual patient following clinical examination.

The present disclosure includes administration regimens in which 2 to 6 loading doses are administered to a patient at a first frequency (e.g., once a week, once every two weeks, once every three weeks, once a month, once every two months, etc.), followed by administration of two or more maintenance doses to the patient on a less frequent basis. For example, according to this aspect of the disclosure, if the loading doses are administered at a frequency of once a month, then the maintenance doses may be administered to the patient once every six weeks, once every two months, once every three months, etc.

### Diagnostic Uses of the Antibodies

The anti-FXI antibodies of the present disclosure may also be used to detect and/or measure FXI, or FXI-expressing cells in a sample, e.g., for diagnostic purposes. For example, an anti-FXI antibody, or fragment thereof, may be used to diagnose a condition or disease characterized by aberrant expression (e.g., over-expression, under-expression, lack of expression, etc.) of FXI. Exemplary diagnostic assays for FXI may comprise, e.g., contacting a sample, obtained from a patient, with an anti-FXI antibody of the disclosure, wherein the anti-FXI antibody is labeled with a detectable label or reporter molecule. Alternatively, an unlabeled anti-FXI antibody can be used in diagnostic applications in combination with a secondary antibody which is itself detectably labeled. The detectable label or reporter molecule can be a radioisotope, such as ³H, ¹⁴C, ³²p, ³⁵S, or ¹²⁵I; a fluorescent or chemiluminescent moiety such as fluorescein isothiocyanate, or rhodamine; or an enzyme such as alkaline phosphatase, beta-galactosidase, horseradish peroxidase, or luciferase. Specific exemplary assays that can be used to detect or measure FXI in a sample include enzyme-linked immunosorbent assay (ELISA), radioimmunoassay (RIA), and fluorescence-activated cell sorting (FACS).

Samples that can be used in FXI diagnostic assays according to the present disclosure include any tissue or fluid sample obtainable from a patient, which contains detectable quantities of FXI protein, or fragments thereof, under normal or pathological conditions. Generally, levels of FXI in a particular sample obtained from a healthy patient (e.g., a patient not afflicted with a disease or condition associated with abnormal FXI levels or activity) will be measured to initially establish a baseline, or standard, level of FXI. This baseline level of FXI can then be compared against the levels of FXI measured in samples obtained from individuals suspected of having a FXI related disease or condition.

### EXAMPLES

The following examples are put forth so as to provide those of ordinary skill in the art with a complete disclosure and description of how to make and use the methods and compositions of the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Generation of Human Antibodies to the CAT Domain of FXI

Human antibodies to the CAT Domain of FXI were generated in a mouse comprising DNA encoding human immunoglobulin heavy and kappa light chain variable regions. In one embodiment, the human antibodies were generated in a VELOCIMMUNE® mouse. In one embodiment, VelocImmune® (VI) mice were immunized with human FXI. The antibody immune response was monitored by FXI specific immunoassay. For example, sera were assayed for specific antibody titers to purified full-length FXI. Antibody-producing clones were isolated using both B-cell Sorting Technology (BST) and hybridoma methods. For example, when a desired immune response was achieved, splenocytes were harvested and fused with mouse myeloma cells to preserve their viability and form hybridoma cell lines. The hybridoma cell lines were screened and selected to identify cell lines that produce FXI-specific antibodies.

Anti-FXI antibodies were also isolated directly from antigen-positive mouse B cells without fusion to myeloma cells, as described in U.S. Pat. No. 7,582,298, herein specifically incorporated by reference in its entirety. Using this method, several fully human anti-FXI antibodies (i.e., antibodies possessing human variable domains and human constant domains) were obtained.

The biological properties of the exemplary antibody generated in accordance with the methods of this Example, controls, and comparators are described in detail in the Examples set forth below.

### Example 2: Heavy and Light Chain Region Sequences

Table 1A sets forth the amino acid sequence identifiers of the heavy and light chain regions of an exemplary anti-FXI antibody of the disclosure. Tables 1B and 1C set forth the nucleic acid (DNA) and amino acid (PEP) sequence identifiers for the heavy and light chain regions of antibodies of the disclosure.

TABLE 1A
Anti-FXI Antibody Sequences for Exemplary
Antibody REGN7508

| | | SEQ |
|---|---|---|
| Antibody | | ID |
| Part | Amino Acid Sequence | NO: |
| Full | QVQLVESGGGLVKPGGSLRL | 18 |
| Length | SCAASGFTFSDYSMNWIRQA | |
| Heavy | PGKGLEWISYISFSGNSIYY | |
| Chain (HC) | ADSVKGRFTISRDNAKNSLY | |
| | LQMNSLRVEDTAIYYCTSRD | |
| | WGYAFDIWGQGTMVTVSSAS | |
| | TKGPSVFPLAPCSRSTSEST | |
| | AALGCLVKDYFPEPVTVSWN | |
| | SGALTSGVHTFPAVLQSSGL | |
| | YSLSSVVTVPSSSLGTKTYT | |
| | CNVDHKPSNTKVDKRVESKY | |
| | GPPCPPCPAPEFLGGPSVFL | |
| | FPPKPKDTLMISRTPEVTCV | |
| | VVDVSQEDPEVQFNWYVDGV | |
| | EVHNAKTKPREEQFNSTYRV | |
| | VSVLTVLHQDWLNGKEYKCK | |
| | VSNKGLPSSIEKTISKAKGQ | |
| | PREPQVYTLPPSQEEMTKNQ | |
| | VSLTCLVKGFYPSDIAVEWE | |
| | SNGQPENNYKTTPPVLDSDG | |
| | SFFLYSRLTVDKSRWQEGNV | |
| | FSCSVMHEALHNHYTQKSLS | |
| | LSLGK | |
| Full | DIQMTQSPSFLSASLGDRVT | 20 |
| Length | ITCQASQDISNYLNWYQVKP | |
| Light | GKAPKLLIYDASNLETGVPS | |
| Chain (LC) | RFSGSGSGTDFTFTISSLQP | |
| | EDIATYFCQQYDNLPYIFGQ | |
| | GTKLEIKRTVAAPSVFIFPP | |
| | SDEQLKSGTASVVCLLNNFY | |
| | PREAKVQWKVDNALQSGNSQ | |
| | ESVTEQDSKDSTYSLSSTLT | |
| | LSKADYEKHKVYACEVTHQG | |
| | LSSPVTKSFNRGEC | |

TABLE 1B
Antibody Heavy Chain Sequences for Exemplary
Antibodies REGN7508 and REGN9932 (SEQ ID NOs)

| | VH | | CDR1 | | CDR2 | | CDR3 | | HC | |
|---|---|---|---|---|---|---|---|---|---|---|
| Antibody | DNA | PEP | DNA | PEP | DNA | PEP | DNA | PEP | DNA | PEP |
| REGN7508 | 1 | 2 | 3 | 4 | 5 | 6 | 7 | 8 | 17 | 18 |
| REGN9932 | 21 | 22 | 23 | 24 | 25 | 26 | 27 | 28 | 37 | 38 |

TABLE 1C
Antibody Light Chain Sequences for Exemplary
Antibodies REGN7508 and REGN9932 (SEQ ID NOs)

| | VK | | CDR1 | | CDR2 | | CDR3 | | LC | |
|---|---|---|---|---|---|---|---|---|---|---|
| Antibody | DNA | PEP | DNA | PEP | DNA | PEP | DNA | PEP | DNA | PEP |
| REGN7508 | 9 | 10 | 11 | 12 | 13 | 14 | 15 | 16 | 19 | 20 |
| REGN9932 | 29 | 30 | 31 | 32 | 33 | 34 | 35 | 36 | 39 | 40 |

The exemplary full length anti-FXI antibody contains fully human Fc gamma 4 heavy chain (i.e., IgG4 Fc) and fully human light chain sequences. However, as will be appreciated by a person of ordinary skill in the art, an antibody having a particular Fc isotype can be converted to an antibody with a different Fc isotype (e.g., an antibody with a mouse IgG1 Fc can be converted to an antibody with a human IgG4, etc.), but in any event, the variable domains (including the CDRs) will remain the same, and the binding properties to antigen are expected to be identical or substantially similar regardless of the nature of the Fc domain.

### Example 3: Biacore Binding Kinetics of Anti-FXI Monoclonal Antibodies Binding to Different FXI Reagents Measured at 25° C. and 37° C.

The goal of this experiment was to determine the kinetics and specificity of hFXI (ERL), hFXIa (ERL), hFXI.mmh (REGN3848) and mfFXI.mmh (REGN3883) binding to anti-FXI mAbs and comparator mAbs at 25° C. and 37° C. The comparator mAbs (REGN6166 or COMP6166) may be found in, for example, U.S. Pat. No. 10,465,011.

### Materials

Instrument used: Biacore 8k and T200-RED
Temp: 25° C. and 37° C.
Running Buffer: HBS—P and 300 mM NaCl, pH7.4
Sensor type: Anti-human Fc mAb (REGN2567)
Flowrate/time: 30 ul/min hFXI association—180 sec hFXI dissociation 600s

### Methods

Approximately 27.1-41.9 RUs of anti-FXI mAbs was captured on anti-hFc mAb (REGN2567) on a CM5 sensor surface. Next, 30 nM stock solutions of hFXI (ERL), hFXIa (ERL), hFXI.mmh (REGN3848) and mfFXI.mmh (REGN3883) were prepared, which were serially diluted 3-fold to make 10 nM, 3.3 nM and 1.1 nM solutions. Then, all FXI solutions were injected on Biacore 8K at 30 μL/min for 180 seconds and dissociation was monitored for 10 minutes.

### Results

TABLE 2
Kinetics of hFXI (ERL) binding to α-FXI mAbs and a comparator mAb at 25° C. and 37° C.

| | 25° C. | | | | | | 37° C. | | | | | |
|---|---|---|---|---|---|---|---|---|---|---|---|---|
| | | hFXI | | | | | | hFXI | | | | |
| | mAb | (ERL) | | | | | mAb | (ERL) | | | | |
| | cap- | bound | | | | | cap- | bound | | | | |
| | tured | (RU) at | ka | kd | K_{D} | t½ | tured | (RU) at | ka | kd | K_{D} | t½ |
| mAb | (RU) | 30 nM | (1/Ms) | (1/s) | (M) | (min) | (RU) | 30 nM | (1/Ms) | (1/s) | (M) | (min) |
| Subject | 27.1 ± | 25.5 | 2.20E+06 | ≤1.00e−5 | 4.54E−12 | ≥1155 | 38.2 ± | 36.7 | 3.24E+06 | ≤1.00e−5 | 3.09E−12 | ≥1155 |
| mAb | 0.1 | | | | | | 0.1 | | | | | |
| [REGN7508] | | | | | | | | | | | | |
| [REGN9932] | 27.6 ± | 6.3 | 8.38E+04 | ≤1.00e−5 | 1.19E−10 | ≥1155 | 36.2 ± | 16.6 | 2.74E+05 | ≤1.00e−5 | 3.65E−11 | ≥1155 |
| | 0.3 | | | | | | 0.1 | | | | | |
| Comp | 28.5 ± | 19.8 | 3.09E+05 | ≤1.00e−5 | 3.24E−11 | ≥1155 | 37.2 ± | 30.9 | 6.22E+05 | 1.83E−05 | 2.95E−11 | 629.5 |
| mAb | 0.2 | | | | | | 0.1 | | | | | |
| [REGN6166] | | | | | | | | | | | | |
| Control | 28.9 ± | −1.0 | NB | NB | NB | NB | 41.6 ± | −1.4 | NB | NB | NB | NB |
| [REGN1945] | 0.1 | | | | | | 0.1 | | | | | |

NB = no binding

At 25° C. and 37° C., anti-FXI mAbs bound human FXI (ERL) with K_{D} values ranging from 4.54 pM-119 pM and 3.09 pM-36.5 pM, respectively.

TABLE 3
Kinetics of hFXIa (ERL) binding to α-FXI mAbs and a comparator mAb at 25° C. and 37° C.

| | 25° C. | | | | | | 37° C. | | | | | |
|---|---|---|---|---|---|---|---|---|---|---|---|---|
| | | hFXIa | | | | | | hFXIa | | | | |
| | | (ERL) | | | | | | (ERL) | | | | |
| | mAb | bound | | | | | mAb | bound | | | | |
| | captured | (RU) at | ka | kd | K_{D} | t½ | captured | (RU) at | ka | kd | K_{D} | t½ |
| mAb | (RU) | 30 nM | (1/Ms) | (1/s) | (M) | (min) | (RU) | 30 nM | (1/Ms) | (1/s) | (M) | (min) |
| Subject | 27.1 ± 0.1 | 27.6 | 3.21E+05 | ≤1.00e−5 | 3.11E−11 | ≥1155 | 37.7 ± 0.2 | 45.9 | 4.75E+05 | 1.21E−04 | 2.56E−10 | 95.1 |
| mAb | | | | | | | | | | | | |
| [REGN7508] | | | | | | | | | | | | |
| [REGN9932] | 27.6 ± 0.1 | −0.6 | NB | NB | NB | NB | 35.9 ± 0.1 | −0.4 | NB | NB | NB | NB |
| Comp mAb | 28.5 ± 0.1 | 2.2 | 2.61E+04 | ≤1.00e−5 | 3.83E−10 | ≥1155 | 37.1 ± 0.2 | 6.4 | 4.48E+04 | 2.96E−04 | 6.61E−09 | 39.0 |
| [REGN6166] | | | | | | | | | | | | |
| Control | 29.0 ± 0.1 | −0.4 | NB | NE | NB | NB | 41.3 ± 0.1 | −0.7 | NB | NB | NB | NB |
| [REGN1945] | | | | | | | | | | | | |

NB = no binding

At 25° C. and 37° C. anti-FXI mAbs bound human FXIa (ERL) with K_{D} values of 31.1 pM and 25.6 pM, respectively.

TABLE 4
Kinetics of hFXI.mmh binding to α-FXI mAbs and a comparator mAb at 25° C. and 37° C.

| | 25° C. | | | | | | 37° C. | | | | | |
|---|---|---|---|---|---|---|---|---|---|---|---|---|
| | | hFXIa | | | | | | hFXIa | | | | |
| | | (ERL) | | | | | | (ERL) | | | | |
| | mAb | bound | | | | | mAb | bound | | | | |
| | captured | (RU) at | ka | kd | K_{D} | t½ | captured | (RU) at | ka | kd | K_{D} | t½ |
| mAb | (RU) | 30 nM | (1/Ms) | (1/s) | (M) | (min) | (RU) | 30 nM | (1/Ms) | (1/s) | (M) | (min) |
| Subject | 27.1 ± 0.1 | 26.8 | 1.87E+06 | ≤1.00e−5 | 5.34E−12 | ≥1155 | 37.5 ± 0.1 | 37.3 | 2.71E+06 | 8.86E−05 | 3.26E−11 | 130.4 |
| mAb | | | | | | | | | | | | |
| [REGN7508] | | | | | | | | | | | | |
| [REGN9932] | 27.6 ± 0.1 | 8.1 | 8.62E+04 | ≤1.00e−5 | 1.16E−10 | ≥1155 | 36.0 ± 0.4 | 19.6 | 2.26E+05 | 8.77E−05 | 3.87E−10 | 131.7 |
| Comp mAb | 28.5 ± 0.1 | 18.4 | 2.87E+05 | ≤1.00e−5 | 3.48E−11 | ≥1155 | 36.9 ± 0.0 | 30.0 | 4.75E+05 | 8.57E−05 | 1.81E−10 | 134.8 |
| [REGN6166] | | | | | | | | | | | | |
| Control | 29.1 ± 0.3 | −0.2 | NB | NB | NB | NB | 40.9 ± 0.1 | −0.5 | NB | NB | NB | NB |
| [REGN1945] | | | | | | | | | | | | |

At 25° C. and 37° C. anti-FXI mAbs bound human FXI.mmh (REGN3848) with K_{D}) values from 5.34 pM-116 pM and 32.6 pM-387 pM, respectively.

TABLE 5
Kinetics of mfFXI.mmh binding to α-FXI mAbs and a comparator mAb at 25° C. and 37° C.

| | 25° C. | | | | | | 37° C. | | | | | |
|---|---|---|---|---|---|---|---|---|---|---|---|---|
| | | hFXIa | | | | | | hFXIa | | | | |
| | | (ERL) | | | | | | (ERL) | | | | |
| | mAb | bound | | | | | mAb | bound | | | | |
| | captured | (RU) at | ka | kd | K_{D} | t½ | captured | (RU) at | ka | kd | K_{D} | t½ |
| mAb | (RU) | 30 nM | (1/Ms) | (1/s) | (M) | (min) | (RU) | 30 nM | (1/Ms) | (1/s) | (M) | (min) |
| Subject | 27.2 ± 0.0 | 28.1 | 2.08E+06 | ≤1.00e−5 | 4.81E−12 | ≥1155 | 37.0 ± 0.1 | 39.1 | 2.56E+06 | 1.11E−04 | 4.35E−11 | 103.7 |
| mAb | | | | | | | | | | | | |
| [REGN7508] | | | | | | | | | | | | |
| [REGN9932] | 27.7 ± 0.2 | 10.8 | 1.55E+05 | ≤1.00e−5 | 6.44E−11 | ≥1155 | 58.7 ± 0.0 | 46.3 | 3.21E+05 | 8.60E−05 | 2.68E−10 | 134.3 |
| Comp mAb | 28.6 ± 0.2 | 22.3 | 3.44E+05 | ≤1.00e−5 | 2.91E−11 | ≥1155 | 36.7 ± 0.1 | 34.5 | 5.15E+05 | 1.09E−04 | 2.11E−10 | 106.3 |
| [REGN6166] | | | | | | | | | | | | |
| Control | 28.9 ± 0.1 | 0.0 | NB | NB | NB | NB | 40.7 ± 0.2 | −0.4 | NB | NB | NB | NB |
| [REGN1945] | | | | | | | | | | | | |

At 25° C. and 37° C., anti-FXI mAbs bound monkey FXI.mmh (REGN3883) with K_{D} values from 4.81 pM-64.4 pM and 43.5 pM-268 pM, respectively.

### Example 4: Activated Partial Thromboplastin Time Bioassay

### Experimental Procedure:

A BIOPHEN Factor XIa kit (HYPHEN BioMed, Neuville-sur-Oise, FR, cat. #220412) was used to assess the capacity of the anti-FXI antibody of disclosure to inhibit the activity of the zymogen Factor FXI (FXI) or pre-activated FXIa leading to the generation of active Factor Xa (FXa). Inhibition by the antibodies of the disclosure was determined by measuring a decrease in the amount of chromogenic substrate converted by FXa (BIOPHEN kit component R3). All the reagents in the BIOPHEN kit were used in the assay except for Reagent 1B (Human Factor IX) and the FXIa calibrator (Cal).

To test the dose-dependent activity of FXI or FXIa, normal human plasma was initially diluted to 0.65% plasma in provided Tris-BSA buffer (used as dilution buffer for assay) and then serially diluted down to 0.021% plasma, along with a no plasma control. Normal human plasma was also diluted to either 0.13% or 0.15% plasma. The antibodies (anti-FXI, controls, and comparators) were serially diluted from a starting concentration of either 500 nM or 300 nM to a concentration of 5.1 pM with a buffer alone sample. For inhibition of zymogen FXI, the anti-FXI antibody was preincubated for 30 minutes at 25° C. with diluted plasma followed by another 30-minute incubation with 0.32 μM aPTT-XL ellagic acid at 25° C. For inhibition of active FXIa, diluted plasma was pre-activated with 0.32 μM aPTT-XL ellagic acid for 30 minutes at 25° C., followed by incubation with the anti-FXI antibody for 30 minutes at 25° C.

After incubations of the plasma with ellagic acid and antibody, Reagent 1A (Human FX, FVIII:C, fibrin polymerization inhibitor) was added and incubated for 5 minutes at 37° C. Then, Reagent 2 (thrombin, phospholipids, and calcium) was added and incubated for 5 minutes at 37° C. Finally, Reagent 3 (SXa-11 FXa substrate) was added and incubated for 30 minutes at 37° C. Absorbance was measured on the FLEXSTATION 3 Plate Reader (Molecular Devices, Sunnyvale, CA) at a wavelength of 405 nm. The results were analyzed using nonlinear regression (4-parameter logistics) with PRISM®6 software (GraphPad, La Jolla, CA) to obtain EC₅₀ and IC₅₀ values. The percent inhibition was calculated based on Equation 2 below:

$$\% Inhibition = 100 \times \frac{{Absorbance}_{Dilute plasma} - {Absorbance}_{Inhibition}}{{Absorbance}_{Dilute plasma} - {Absorbance}_{No Plasma control}}$$

In this equation, “Absorbance Dilute plasma” refers to the absorbance measurement at 405 nm of dilute plasma (either 0.13% or 0.15% plasma) that has been activated with 0.32 μM aPTT-XL Ellagic Acid to cleave FXI to FXIa without any added antibody. “Absorbance Inhibition” refers to the minimum absorbance measurement at 405 nm from a dose response of a particular antibody with dilute plasma, activated by 0.32 μM Ellagic Acid. “Absorbance No Plasma control” refers to the absorbance measurement at 405 nm of Tris-BSA buffer alone in the absence of any plasma.

### Tabulated Data Summary:

TABLE 6
Anti-FXI/FXIa antibody inhibition of FXI activation to FXIa
or preactivated FXIa activity in aPTT-XL Ellagic Acid Assay.

| | 0.13% Plasma | | | | 0.15% Plasma | | | |
|---|---|---|---|---|---|---|---|---|
| | FXI | | FXIa | | FXI | | FXIa | |
| | % | IC50 | % | IC50 | % | IC50 | % | IC50 |
| Antibody | Inhibition | [M] | Inhibition | [M] | Inhibition | [M] | Inhibition | [M] |
| [REGN9932] | 87 | 1.9E−10 | 35 | >1.0E−08 | not | not | not | not |
| | | | | | tested | tested | tested | tested |
| Comp mAb | not | not | not | not | 108 | 3.8E−11 | 95 | 4.8E−10 |
| [REGN6166] | tested | tested | tested | tested | | | | |
| Control | 58 | >1.0E−07 | no | no | 102 | 1.2E−07 | no | no |
| [REGN1945] | | | inhibition | inhibition | | | inhibition | inhibition |

### Results Summary and Conclusions:

As shown in Table 6, the anti-FXI/FXIa antibody showed inhibition of FXI in dilute normal plasma with an IC50 value of 190 PM and with maximum inhibition ranging of 87%. The anti-FXI/FXIa antibody of the disclosure also inhibited FXIa in dilute plasma with an IC50 value of to greater than 10 nM and with maximum inhibition of 35%. The comparator mAb showed inhibition of FXI with an IC50 value of 38 pM and with maximum inhibition of 108%. Comparator mAb also showed inhibition of FXIa with an IC50 value of 480 pM with maximum inhibition of 95%. Isotype Control mAb showed no inhibition of FXIa, but showed inhibition of FXI at high concentration of the antibody with IC50 values ranging from >100 nM-120 nm with maximum inhibition ranging from 58-102% inhibition.

### Example 5: Complex Formation

### 5.1: Size Analysis of Complexes Formed Between Human Coagulation Factor XI and the Subject mAb [REGN7508]

### Experimental Conditions

### Sample Preparation

Solutions of anti-hFXI mAbs with hFXI were prepared at equimolar ratio and allowed to incubate at room temperature for 2 hr. Following incubation, complex solutions were injected into the column.

### SEC-MALS Conditions

Samples were fractionated on a tandem Waters ACQUITY UPLC BEH®200 SEC column (1.7 μm, 4.6 mm×150 mm) columns that was pre-equilibrated in 10 mM sodium phosphate, 500 mM sodium chloride, pH 7.0 with a flow rate of 0.3 mL/min.

Protein species eluting from the column were monitored by 3 in-line detectors: an absorbance detector (280 nm), multi-angle light scattering (MALS) detector, and a refractive index detector.

### Data Analysis

Molar mass was determined via protein conjugate analysis for the free ligand and free drug samples. Complex samples used modified dn/dc and UV values determined from protein conjugate analysis of free samples.

Data analysis was performed using Astra™ software version 7.3.1.9.

### Results

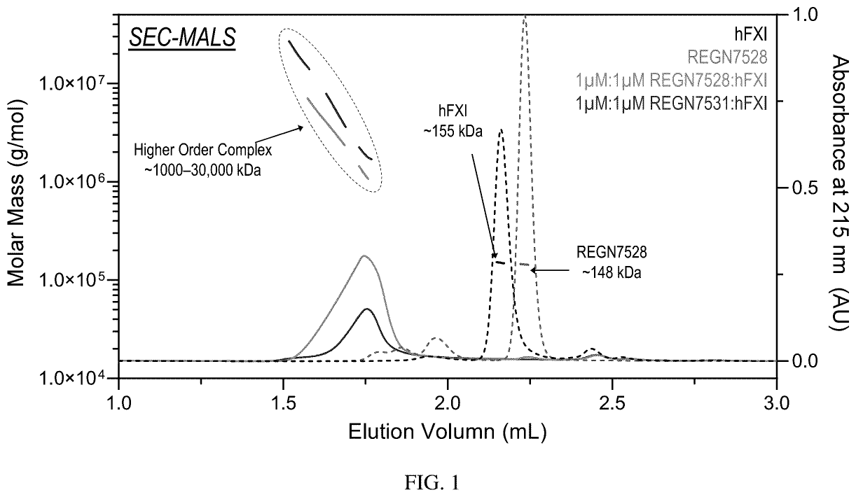

Overall, as seen in FIG. 1, REGN7528 and REGN7531 displayed the clearest evidence of extensive “paper-dolling”.

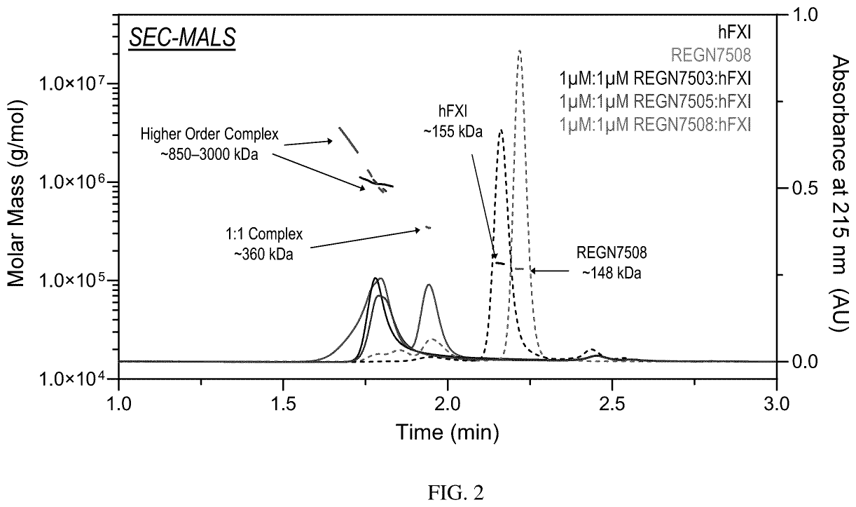

In comparison, and as seen in FIG. 2, REGN7503 and REGN7505 appeared to form smaller, less heterogeneous complexes; only the subject mAb [REGN7508] displayed existence of a 1:1 complex with FXI.

TABLE 7
Overall Avg Mw (kDa) from SEC-MALS

| | | Overall Avg |
|---|---|---|
| Tested | | Mw (kDa) from |
| Articles | Description | SEC-MALS |
| [REGN7503] | Anti-hFXI mAb, A3/A4 domain binder | 1087 |
| [REGN7505] | Anti-hFXI mAb, A3/A4 domain binder | 1081.1 |
| Subject mAb | Anti-hFXI mAb, catalytic domain | 1135.1 |
| [REGN7508] | binder | |
| [REGN7528] | Anti-hFXI mAb, catalytic domain | 2354 |
| | binder | |
| [REGN7531] | Anti-hFXI mAb, A3/A4 domain binder | 4103.4 |

### Discussion

SEC-MALS was used to quickly assess the relative size distribution of complexes formed between human Coagulation Factor XI (hFXI) and a panel of anti-hFXI mAbs in order to eliminate mAbs that displayed extensive “paper-dolling” and provide additional characterization to support a time-sensitive decision.

SEC-MALS analysis was chosen for expediency, but due to the limited resolution range of the column, a detailed interpretation of stoichiometry was prohibited; only qualitative comparisons can be made from this data.

Overall, out of the panel of mAbs, REGN7528 (Catalytic domain) and REGN7531 (A3/A4 domain) displayed the clearest evidence of extensive “paper-dolling”.

In comparison, REGN7503 (A3/A4 domain) and REGN7505 (A3/A4 domain) appeared to form a more homogeneous distribution of complexes with smaller overall average molar mass; however, subject mAb [REGN7508] (Catalytic domain) was the only mAb out of the panel to display a prominent 1:1 complex with FXI as previously observed by A4F-MALS.

5.2 Size Analysis of Complexes Formed Between human Coagulation Factor XI and the Subject mAb [REGN7508]

### Experimental Conditions

### Sample Preparation

The samples were prepared in 1× DPBS, pH 7.4 and allowed to incubate at room temperature for 2 hrs. prior to fractionation of total protein by A4F-MALLS (PostNova).

### A4F-MALLS Conditions

7 mg complex or 4 mg mAb or ligand was injected onto an A4F short channel fitted with a 350 W spacer and a 10 kDa Regenerated Cellulose membrane with a mobile phase of 10 mM sodium phosphate, 500 mM NaCl, pH 7.0 and separated using a gradient as below. Flow Rates:
Channel Flow: 1.0 mL/min
Focus Flow: 1.0 mL/min for 4 min.
Cross Flow: 3.0 mL/min to 0 mL/min linear gradient over 45 min, followed by 0 mL/min for 10 min.

### Data Analysis

Molar mass was determined via protein conjugate analysis for the free ligand and free antibody samples. Complex samples used modified dn/dc and UV values determined from protein conjugate analysis of free samples.

### Results

TABLE 8
Subject mAb:FXI Complex and Theoretical Molar Mass

| | Theoretical |
|---|---|
| mAb:FXI Complex | Molar Mass (kDa) |
| 1:0 | 151 |
| 0:1 | 155 |
| 1:1 | 306 |
| 1:2¹ | 461 |
| 2:2 | 612 |
| 2:3¹ | 767 |
| 3:3 | 918 |
| 3:4¹ | 1073 |
| 4:4 | 1224 |

¹Unequal ratios (such as 1:2 and 2:1) cannot be differentiated, because they will have similar MW

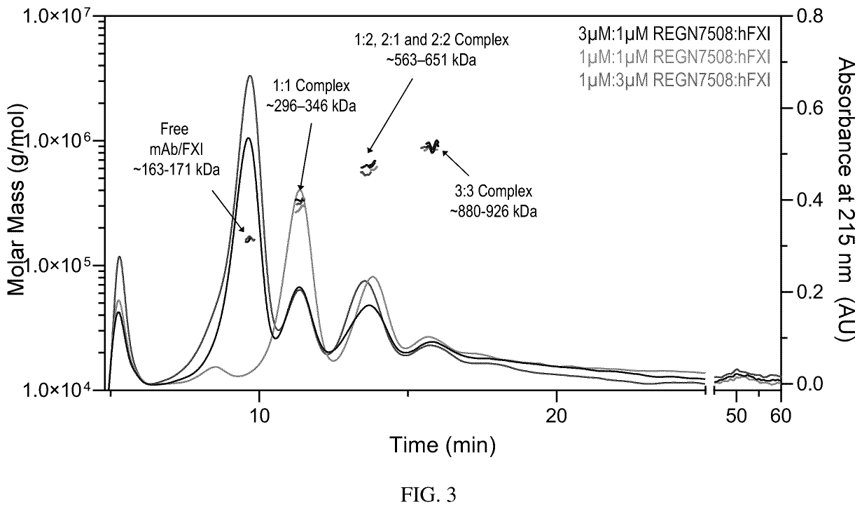

As seen in Table 8 (above) and in FIG. 3, the subject mAb [REGN7508] (Catalytic domain) favored lower-order complexes with the predominant species representing a discrete 1:1 and 2:2 complex with hFXI.

TABLE 9
mAb:FXI Complex and Theoretical Molar Mass

| | Theoretical |
|---|---|
| mAb:FXI Complex | Molar Mass (kDa) |
| 1:0 | 148 |
| 0:1 | 155 |
| 1:1 | 303 |
| 1:2¹ | 458 |
| 2:2 | 606 |
| 2:3¹ | 761 |
| 3:3 | 909 |
| 3:4¹ | 1064 |
| 4:4 | 1212 |

¹Unequal ratios (such as 1:2 and 2:1) cannot be differentiated, because they will have similar MW

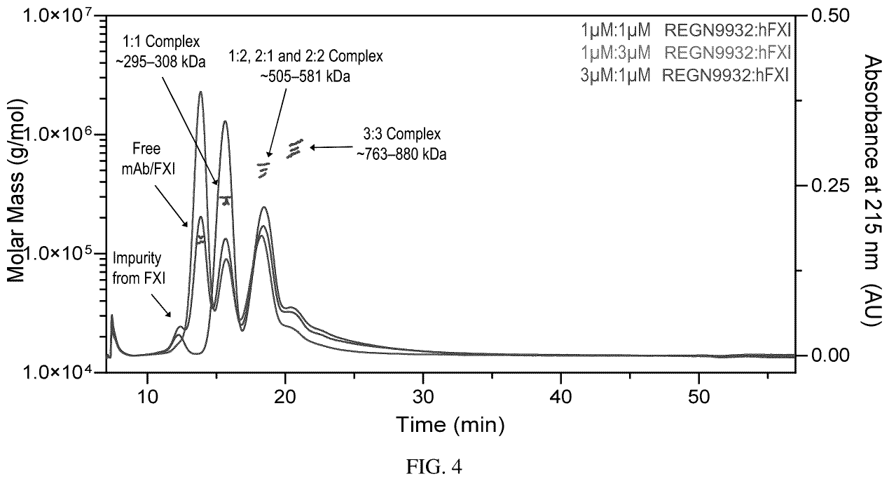

As seen in Table 9 (above) and in FIG. 4, [REGN9932] (Catalytic domain) formed the highest proportion of lower-order complexes with the predominant species representing a discrete 1:1 and 2:2 complex with hFXI.

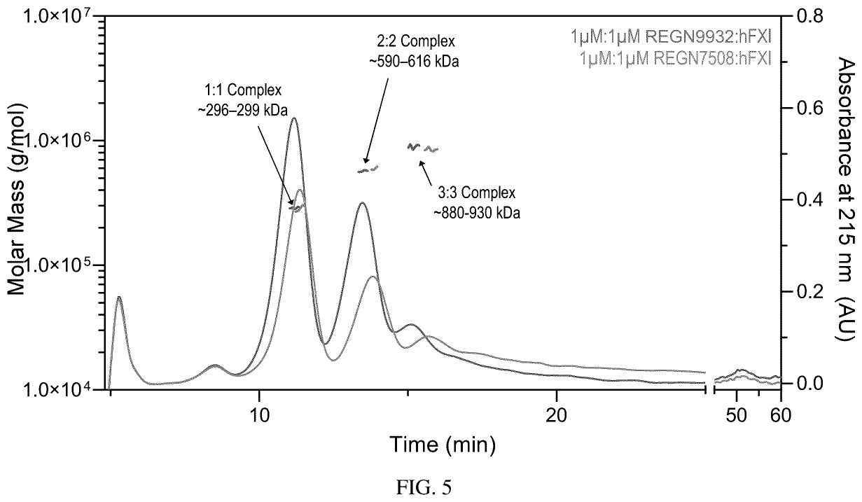

As seen in FIG. 5, the molar mass and distribution of complexes formed between the subject mAb [REGN7508] (Catalytic domain) and hFXI was comparable to those previously observed with [REGN9932] (Catalytic domain).

### Discussion

Asymmetric flow field-flow fractionation coupled to multi-angle laser light scattering (A4F-MALLS) was used to assess the relative size distribution of complexes formed between human Coagulation Factor XI (hFXI, from Enzyme Research Laboratory) and several anti-hFXI mAbs (the subject mAb [REGN7508] and [REGN9932]),

The subject mAb [REGN7508] (Catalytic domain) favored lower-order complexes with the predominant species representing a discrete 1:1 and 2:2 complex with hFXI when mixed at varying molar ratios.

Molar mass and size distribution observed for complexes formed between the subject mAb [REGN7508] (Catalytic domain) and hFXI was comparable to those previously observed with [REGN9932] (Catalytic domain).

### Example 6: Thrombin Generation Assays (TGA)

TGAs were conducted to measure the following endpoints:
Lag Time (min), clotting time
Peak of Thrombin (nM)
ttPeak: Time to Peak (min)
ETP: Endogenous Thrombin Potential (nM*min) (ETP; area under curve)
Velocity: Peak(nM)/ttPeak(min)

### 6.1: TGA Profiles in Pooled Human Plasma

### Results

TABLE 10
anti-FXI/FXIa Testign Profiles in Pooled Human Plasma

| | | | | | | | | | | TGA- | TGA- |
|---|---|---|---|---|---|---|---|---|---|---|---|
| | | | | | | | | | | TF: % of | TF: % of |
| | | | TGA-EA | | TGA-EA | | TGA-EA | | TGA-TF | ETP w/ | Peak w/ |
| mAb | aPTT-C₂ₓₜ | PT-C₂ₓₜ | C_{2x LagT} | | C_{1/2 ETP} | | C_{1/2 Peak} | | C_{2x LagT} | 500 nM | 500 nM |
| Comp mAB | 26 nM | NC | 31 | nM | >500 | nM | 500 | nM | NC | 66 | 65 |
| [COMP6166] | | | | | | | | | | | |
| [REGN9932] | 32 nM | NC | 31 | nM | 62 | nM | 31 | nM | NC | 68 | 70 |
| Subject mAb | 22 | NC | 16-31 | nM | 16-31 | nM | 16-31 | nM | NC | 52 | 53 |
| [REGN7508] | | | | | | | | | | | |

NC = No change

### 6.2: TGA Profiles in Cyno Monkey Plasma (Female)

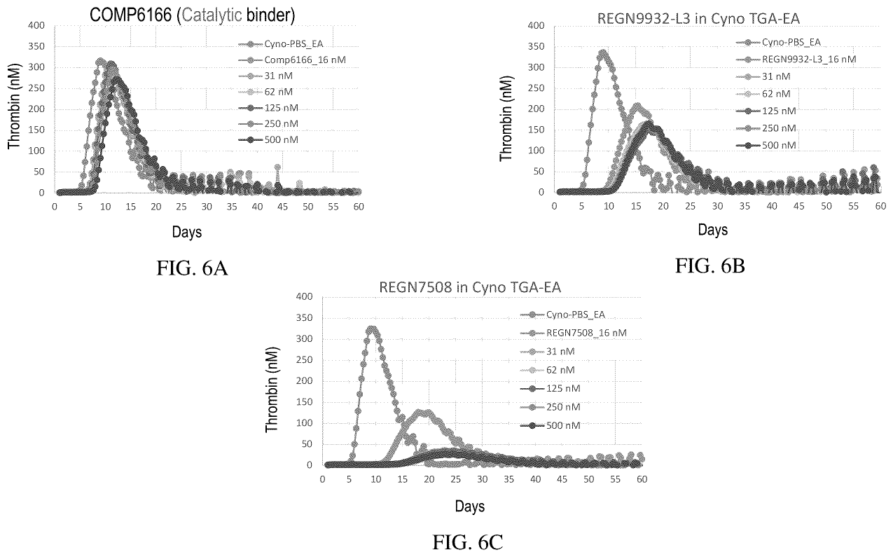

A comparison of the effects of anti-FXI mAbs on the intrinsic pathway thrombin generation in Cyno Monkey Plasma (Female) can be seen in FIGS. 6A, 6B, 6C.

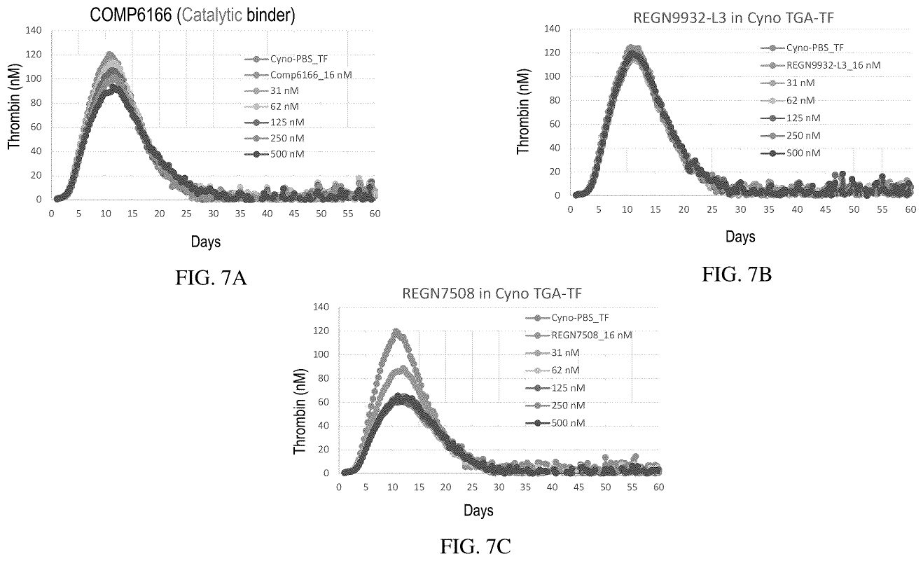

A comparison of the effects of anti-FXI mAbs on the intrinsic pathway thrombin generation in Cyno Monkey Plasma (Female) can be seen in FIGS. 7A, 7B, 7C.

### 6.3: TGA Profiles in Pooled Women Plasma

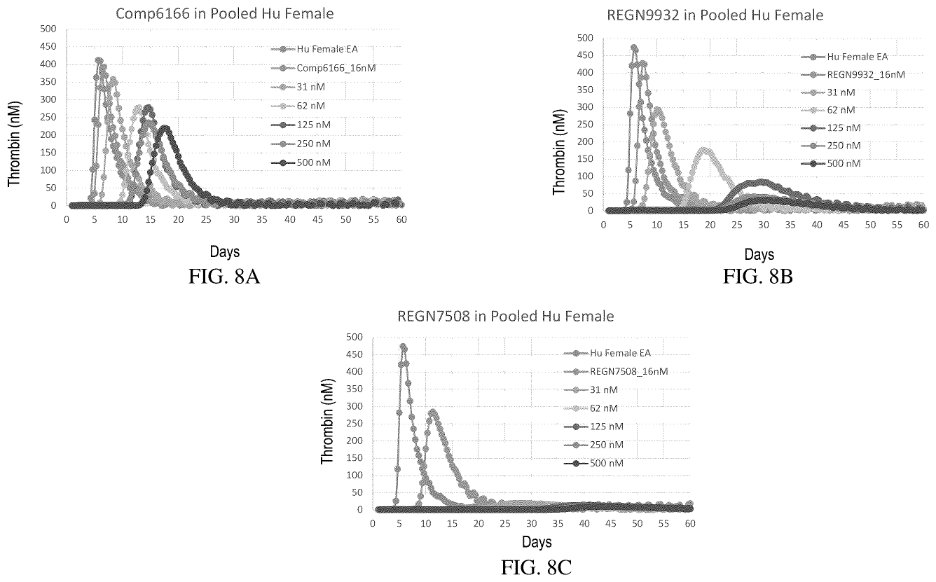

A comparison of the effects of anti-FXI mAbs on the intrinsic pathway thrombin generation in Pooled Women Plasma can be seen in FIGS. 8A, 8B, 8C.

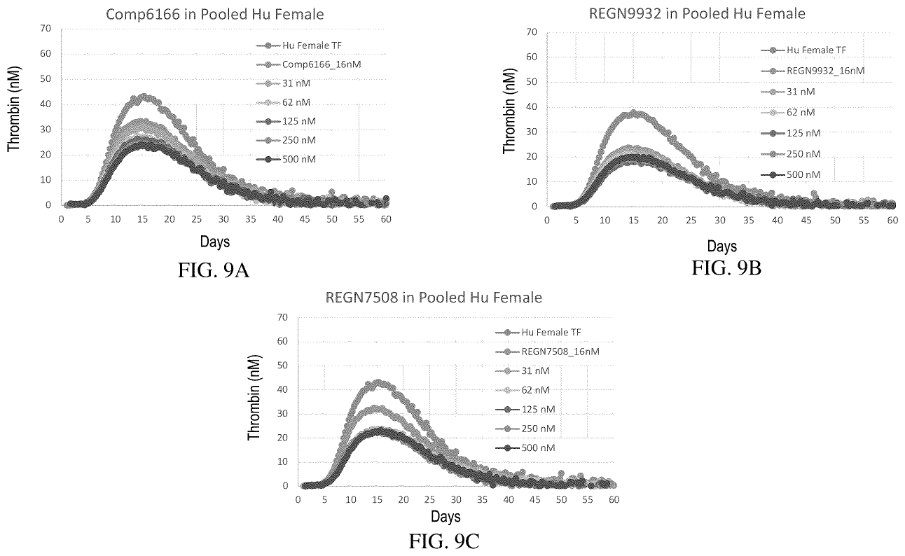

A comparison of the effects of anti-FXI mAbs on the intrinsic pathway thrombin generation in Pooled Women Plasma can be seen in FIGS. 9A, 9B, 9C.

6.4: Comparison of Anti-FXI/FXIa mAbs on Thrombin Generation in Six Single Donors

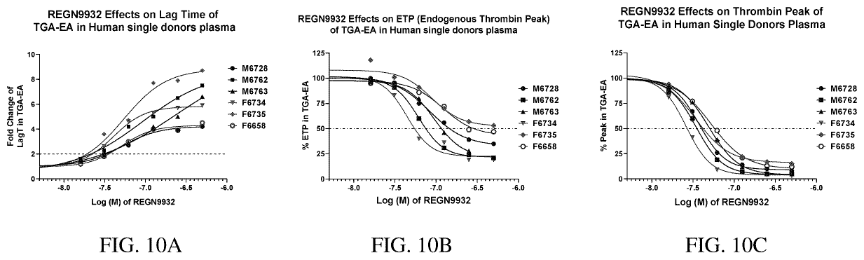

A comparison of the effects of anti-FXI mAb [REGN9932] on the intrinsic pathway thrombin generation in Six Single Donors can be seen in FIGS. 10A, 10B, 10C.

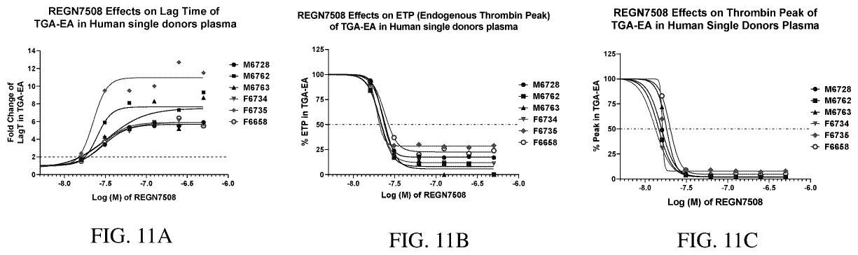

A comparison of the effects of the subject anti-FXI mAb [REGN7508] on the intrinsic pathway thrombin generation in Six Single Donors can be seen in FIGS. 11A, 11B, 11C.

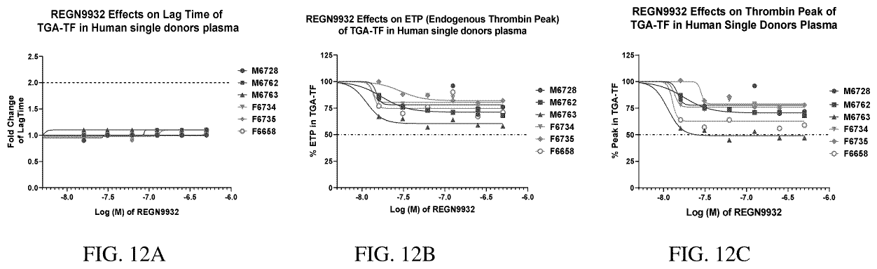

A comparison of the effects of anti-FXI mAb [REGN9932] on the extrinsic pathway thrombin generation in Six Single Donors can be seen in FIGS. 12A, 12B, 12C.

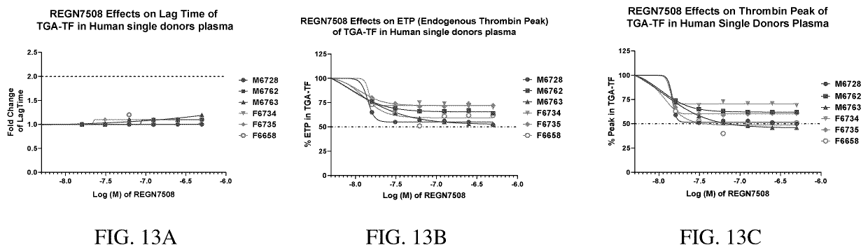

A comparison of the effects of the subject anti-FXI mAb [REGN7508] on the extrinsic pathway thrombin generation in Six Single Donors can be seen in FIGS. 13A, 13B, 13C.

### Example 7: Pharmacokinetic Study of Subject Anti-FXI Antibody Drug Substance in Cynomolgus Monkeys

The objective of this study is to determine the intravenous single-dose pharmacodynamic/pharmacokinetic (PK/PD) parameters of anti-FXI monoclonal antibodies (mAbs) in cynomolgus monkeys over a period of 8 weeks.

Plasma is collected at the following time points: Pre-dose, 5 min, 6 hr, Day 1, Day 2, Day 3, Day 5, Day 7, Day 14, Day 21, Day 28, Day 35, Day 42, Day 49, and Day 56.

The measured endpoints are as following:
1) Target level (total)
2) hFc level (total)
3) aPTT/PT
4) TGA-EA/TF
5) Modified FXIa activity assay (Biophen)
6) CBC (starting at 72 hrs)

TABLE 11
Summary of Doses and Dose Groups

| | Domain | | Dose | “n” per |
|---|---|---|---|---|
| mAb | Specificity | Route | (mg/kg) | dosing group |
| Control: | | IV | | 3 |
| Bleeds and recycle | | | | |
| Comp mAb | Catalytic | IV | 1 | 3 |
| [COMP6166] | | | 3 | 3 |
| | | | 10 | 3 |
| Subject mAb | Catalytic | IV | 1 | 3 |
| [REGN7508] | | | 3 | 3 |
| | | | 10 | 3 |
| [REGN9932*] | Catalytic | IV | 1 | 3 |

*REGN9932 will be used as the “bridge” from a previous PK/PD study

### Example 8: Determination of Kinetic and Equilibrium Binding Parameters for the Interaction of Subject Anti-FXI Antibody Drug Substance with FXI from Human, Cynomolgus Monkey, Rabbit, and Mouse and FXIa from Human

### 8.1: Experimental Design

Surface plasmon resonance (SPR) experiments were performed on a Biacore instrument to determine the binding affinity of REGN7508 for human FXI and FXIa proteins derived from plasma and to recombinant mmH-tagged forms of human, cynomolgus monkey, rabbit, and mouse FXI proteins. Varying concentrations of plasma-derived and recombinant FXI or FXIa proteins were injected over sensor surface-captured REGN7508 at pH 7.4 and 25° C. (all FXI and FXIa proteins) or 37° C. (plasma-derived human FXI/FXIa only), followed by a dissociation phase. Binding signal changes were recorded, specific binding signals were calculated, and kinetic binding parameters were determined by fitting the data to a 1:1 binding model with mass transport limitation.

### Covalent Coupling of Anti-Human FcG Antibody to the Sensor Chip Surface

Mouse anti-human FcG monoclonal antibody (anti-hFcG) was immobilized on the surface of a sensor chip using standard amine-coupling chemistry. The coupling procedure was performed using filtered and degassed HBS-P (10 mM HEPES, 300 mM NaCl, 0.05% (v/v) Polysorbate 20, pH 7.4) as running buffer at a flow rate of 10 μL/min. The sensor surfaces were activated by injecting a 1:1 (by volume) mixture of 0.4M 1-ethyl-3-(3-dimethylaminopropyl) carbodiimide hydrochloride and 0.1M N-hydroxysuccinimide over the chip for 7 minutes. Following surface activation, anti-hFcG (20 μg/mL) prepared in 10 mM sodium acetate, pH 5.0, was injected over the activated chip surfaces for 7 minutes. The remaining active groups on the sensor chip surface were blocked by injecting 1M ethanolamine for 7 minutes until a final surface density of approximately 1,900 resonance units (RU) was reached. The sensor chip surfaces were then treated with at least 10 injections of 20 mM phosphoric acid for 12 seconds each to remove all uncoupled residual proteins and washed with running buffer HBS-P prior to performing the kinetic binding experiments.

Kinetic Binding Interactions of REGN7508 with FXI and FXIa Proteins

The binding of FXI and FXIa proteins to REGN7508 (lot #9048800001 [all FXI/FXIa proteins] and lot #REGN7508-L2 [human FXI/FXIa only]) was measured at pH 7.4 and 25° C. or 37° C., using HBS-P as running buffer. REGN7508 was captured by surface-coupled anti-hFcG until a signal of 59-101 RU was reached. Plasma-derived and recombinant FXI and FXIa proteins were individually injected over the captured REGN7508 surfaces in 2-fold serial dilutions at concentrations ranging from 0.781 nM to 25.0 nM (hFXI, hFXIa, hFXI.mmH or MfFXI.mmH), 7.81 nM to 250 nM (rbFXI.mmH), and 1.56 nM to 50 nM (mFXI.mmH) at a flow rate of 50 μL/min for 1 minute (25° C.) or 0.5 minutes)(37° C. followed by a 20-minute dissociation phase, and the resultant binding signal changes were recorded. Each concentration was tested in duplicate.

Specific binding signals were obtained by a double referencing procedure and plotted as SPR sensorgrams. The double referencing was performed by first subtracting the signal of each injection over a reference surface (anti-hFcG) from the signal over the experimental surface (anti-hFcG-captured REGN7508) thereby removing contributions from refractive index changes. In addition, running buffer injections were performed to allow subtraction of the signal changes resulting from the dissociation of captured REGN7508 from the coupled anti-hFcG surface. The kinetic parameters were obtained by globally fitting these specific binding signals to a 1:1 binding model with mass transport limitation. The equilibrium dissociation constant (K_{D}) was calculated from the ratio of the dissociation rate constant to the association rate constant (K_{D}=kd/ka). The dissociative half-life (t½) was calculated by dividing 0.693 (natural logarithm of 2) by the experimentally determined kd.

### 8.2: Results

Binding Parameters for the Interaction of REGN7508 with FXI and FXIa

Kinetic binding parameters for the interaction of REGN7508 with FXI proteins from human, cynomolgus monkey, rabbit, and mouse and with FXIa from human were determined using SPR technology. Human FXI (E19-V625) shares 96%, 86%, and 79% amino acid sequence identity with cynomolgus monkey, rabbit, and mouse FXI, respectively. These assays used injections of FXI or FXIa proteins at a range of concentrations over captured REGN7508 sensor surfaces at 25° C. and pH 7.4. The calculated kinetic binding parameters are summarized in Table 12.

In SPR experiments performed at 25° C. and pH 7.4, REGN7508 bound plasma-derived hFXI and hFXIa, as well as recombinant human FXI (hFXI.mmH) with picomolar affinities.

REGN7508 also bound recombinant cynomolgus monkey FXI (MfFXI.mmH) with picomolar affinity. REGN7508 did not bind recombinant rabbit (rbFXI.mmH) or mouse (mFXI.mmH) proteins up to the highest concentration tested (250 nM or 50 nM respectively), indicating binding specificity for human and cynomolgus monkey FXI.

The kinetic binding parameters for the interaction of a separate lot of REGN7508 with human FXI and FXIa were also determined. REGN7508 lot #9048800001, described above and used in the toxicology studies, and REGN7508 lot #REGN7508-L2, used in several nonclinical in vitro and in vivo pharmacology studies, showed similar binding affinities for human FXI and FXIa.

TABLE 12
Summary of Kinetic Binding Parameters for the Interaction
of REGN7508 with FXI and FXIa at 25° C. and pH 7.4

| Protein Injected | Kinetic Binding Parameters | | | |
|---|---|---|---|---|
| Over Surface- | kₐ | k_{d} | K_{D} | t_{1/2} |
| Captured REGN7508 | (M⁻¹s⁻¹) | (s⁻¹) | (M) | (min) |
| hFXI (plasma-derived) | 3.12E+06 | 9.36E−06 | 3.00E−12 | 1234 |
| hFXIa (plasma-derived) | 3.55E+06 | 3.61E−05 | 1.02E−11 | 320 |
| hFXI.mmH | 3.34E+06 | 2.70E−05 | 8.07E−12 | 428 |
| MfFXI.mmH | 2.58E+06 | 9.62E−06 | 3.73E−12 | 1201 |

| | |
|---|---|
| rbFXI.mmH | NB |
| mFXI.mmH | NB |

ka, association rate constant; kd, dissociation rate constant; KD, equilibrium dissociation constant; t½: dissociative half-life; NB, No detectable binding observed under the assay conditions used.

The REGN7508 equilibrium dissociation constants (KD) for plasma-derived human FXI and FXIa were 3.00 and 10.2 pM, respectively. REGN7508 also bound recombinant human (hFXI.mmH) and cynomolgus monkey (Macaca fascicularis) (MfFXI.mmH) FXI with KD values of 8.07 and 3.73 pM, respectively, but did not show detectable binding to recombinant rabbit (rbFXI.mmH) or mouse (mFXI.mmH) FXI proteins up to the highest concentration tested (250 and 50 nM respectively).

### Example 9: In Vitro Functional Characterization of REGN7508

### 9.1: Experimental Design

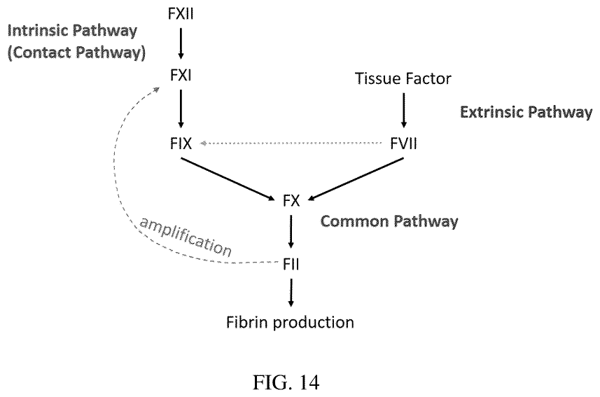

The capacity of REGN7508 to block the coagulation pathway in human and cynomolgus monkey donor plasma was assessed in vitro using clotting assays and TGAs. Effects on the intrinsic coagulation pathway were measured based on aPTT and thrombin generation induced by EA, while effects on the extrinsic coagulation pathway were measured by PT and thrombin generation induced by TF (FIG. 14).

The aPTT test evaluates all clotting factors of the intrinsic and common pathways of the coagulation cascade by measuring time for a clot to form after the addition of calcium and EA. The PT test evaluates all clotting factors of the extrinsic and common pathways of the coagulation cascade after the addition of calcium and TF.

The TGA induced by EA measures the rate and amount of thrombin generated via the intrinsic and common pathways. The TGA induced by TF measures the rate and amount of thrombin generated via the extrinsic and common pathways.

aPTT Assay (for Intrinsic Pathway Activity)

The aPTT in the presence of REGN7508 or an IgG4P isotype control in either human or cynomolgus monkey donor plasma was determined using a STart4 Hemostasis Analyzer. Plasma sample (50 μL) was added to a STart® Cuvette and incubated with 2-fold serial dilutions of either REGN7508 (9 nM to 1.2 μM) or an IgG4P isotype control (19 nM to 1.2 μM) at 37° C. for 5 min; a no-antibody control cuvette containing PBS in lieu of antibody was also included for baseline measurement. A second set of REGN7508 concentrations was also tested at increments of 4 nM from 4 nM up to 28 nM and 31 nM. 50 μL of aPTT-XL EA was added for a 5-minute incubation followed by the addition of 50 μL of 20 mM calcium chloride to start the reaction. The measured clotting time for each test sample was normalized to the plasma clotting time of the no-antibody control. The average change relative to the no-antibody control for each concentration (run in duplicate) was plotted against the antibody concentrations.

The concentration at which aPTT is doubled (doubling time) is C2xt and it was determined in the second set of experiments where a range of antibody concentrations (4 nM to 31 nM) were tested with smaller increments of 4 nM. In aPTT clotting time, the values in seconds were generated relative to baseline ie, no-antibody control or PBS only value, that was equivalent to 1.0-fold change in aPTT. C2xt was estimated at the intersection of the ‘Doubling Time Line’ and the aPTT curve. The doubling time line would be placed at twice the value of baseline in seconds or equivalent to 2.0 in fold change of aPTT for the mAb value, relative to baseline in GraphPad Prism.

### PT Assay (for Extrinsic Pathway Activity)

The PT in the presence of REGN7508 or an IgG4P isotype control in either human or cynomolgus monkey donor plasma was determined using a STart4 Hemostasis Analyzer. Plasma sample (50 μL) was added to a STart® Cuvette and incubated with either REGN7508 or an IgG4P isotype control (600 nM and 1.2 μM) at 37° C. for 5 min; a no-antibody control plasma sample containing PBS in lieu of antibody, was also included for baseline measurement. TriniCLOT PT Excel S (TF and calcium, 100 μL) was added to start the reaction. The measured clotting time for each test sample was normalized to the plasma clotting time of the no-antibody control. The average change relative to the no-antibody control for each concentration (run in duplicate) was plotted against the antibody concentrations.

### Thrombin Generation Assays

The thrombin generation profiles for REGN7508 or an IgG4P isotype control in either human or cynomolgus monkey donor plasma were determined using a Calibrated Automated Thrombogram® platform. Thrombin activity was measured by monitoring the splitting of a fluorogenic substrate and comparing it to a constant known thrombin activity in a non-clotting sample evaluated in parallel. Plasma sample (55 μL) was added to a well of a Immulon II HB U Bottom Microplate and incubated with 2-fold serial dilutions of either REGN7508 or an IgG4P isotype control ranging from 16 nM to 500 nM at 37° C. for 30 min; a no-antibody control well containing PBS in lieu of antibody was also included for baseline measurement. A second set of REGN7508 concentrations was also tested for intrinsic pathway activity only at increments of 4 nM from 4 nM to 31 nM; an IgG4P isotype control was tested at 31 nM.

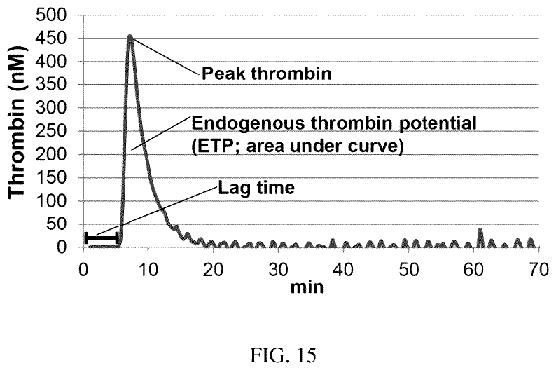

Thrombin generation was then induced by addition of either 15 μL aPTT-XL EA pre-diluted in MP reagent (intrinsic pathway activity) or 15 μL PPP Reagent Low TF (extrinsic pathway activity). After incubation for 45 minutes at 37° C., 15 μL of pre-warmed Fluo substrate in FluCa buffer was added to the wells immediately before a continuous 90-minute reading of the Immulon II HB U Bottom Microplate. The measured real-time thrombin concentration values recorded over the first 60 minutes were plotted against time to yield a thrombogram profile for each antibody concentration tested (FIG. 15). The lag time, peak thrombin, and endogenous thrombin potential were determined from each thrombogram.

### 9.2: Results

### 9.2.1: Effect of REGN7508 in Clotting Assays With Human or Cynomolgus Monkey

### Donor Plasma

Clotting Assays with Human Plasma

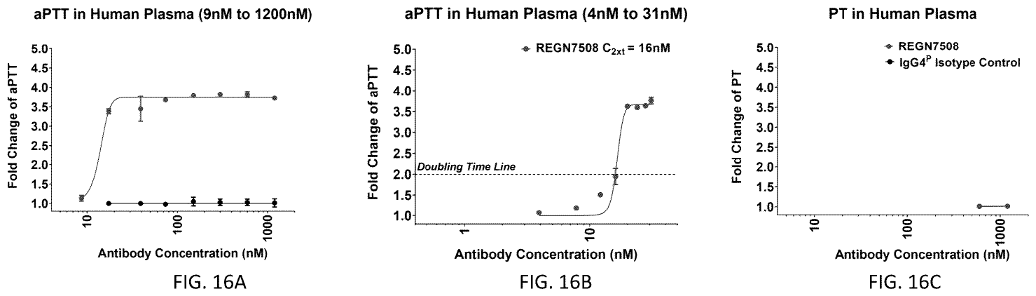

REGN7508 increased aPTT relative to baseline (no antibody) in a concentration-dependent manner; with up to 3.8-fold increase in human plasma at concentrations from 9 nM to 1.2 pM (FIG. 16A and Table 13) as well as at concentrations from 4 nM to 31 nM tested in a second set of experiments (FIG. 16B and Table 14). The doubling of aPTT relative to baseline was estimated to occur at 16 nM in human plasma, when tested in the second set of experiments using an antibody concentration range with smaller increments (4 nM to 31 nM). No change in PT relative to baseline was observed in human (FIG. 16C and Table 13) plasma at the highest antibody concentration tested (1.2 μM). No changes in either aPTT or PT relative to baseline were observed for the IgG4P isotype control in human plasma up to the highest antibody concentration tested (1.2 μM).

TABLE 13
Summary of aPTT (9 nM to 1.2 μM) and PT (600
nM and 1.2 μM) in Human Donor Plasma

| | Change in aPTT^{a} | | Change in PT^{a} | |
|---|---|---|---|---|
| Antibody | | IgG4^{P} | | IgG4^{P} |
| Concentration | | Isotype | | Isotype |
| (nM) | REGN7508 | Control | REGN7508 | Control |
| 9 | 1.13 | NT | NT | NT |
| 19 | 3.38 | 1.00 | | |
| 38 | 3.45 | 1.00 | | |
| 75 | 3.68 | 0.98 | | |
| 150 | 3.79 | 1.05 | | |
| 300 | 3.82 | 1.03 | | |
| 600 | 3.81 | 1.03 | 1.01 | 1.01 |
| 1200 | 3.72 | 1.01 | 1.02 | 1.02 |

^{a}Relative to baseline (ie, no antibody);
NT, Not tested

TABLE 14
Summary of aPTT in Human Donor Plasma (4 nM to 31 nM)

| | |
|---|---|
| Antibody | Change in aPTT^{a} |

| Concentration (nM) | REGN7508 | Ig4^{P} Isotype Control |
|---|---|---|
| 4 | 1.08 | NT |
| 8 | 1.19 | |
| 12 | 1.51 | |
| 16 | 1.95 | |
| 20 | 3.63 | |
| 24 | 3.60 | |
| 28 | 3.63 | |
| 31 | 3.76 | |

^{a}Relative to baseline (ie, no antibody);
NT, Not tested

Clotting Assays with Cynomolgus Monkey Plasma

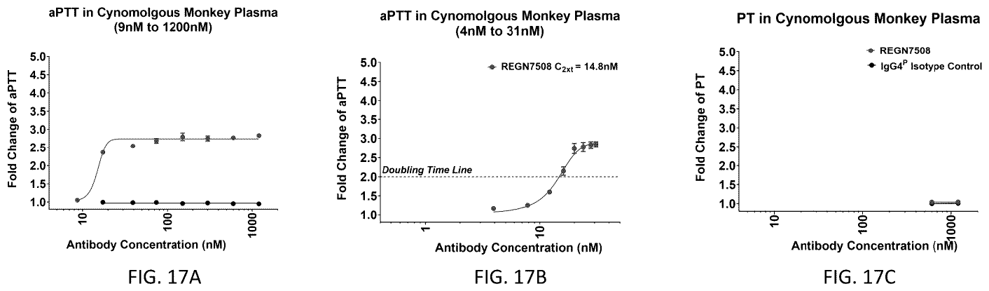

REGN7508 increased aPTT relative to baseline (no antibody) in a concentration-dependent manner; with up to 2.8-fold increase in cynomolgus monkey plasma, at concentrations from 9 nM to 1.2 μM (FIG. 17A and Table 15) as well as in a second set of concentrations tested from 4 nM to 31 nM (FIG. 17B and Table 16). In a second experiment, using an antibody concentration range with smaller increments (4 nM to 31 nM), the doubling of aPTT relative to baseline was estimated to occur at 14.8 nM in cynomolgus monkey plasma. No change in PT relative to baseline was observed in cynomolgus monkey (FIG. 17C and Table 15) plasma at the highest antibody concentration tested (1.2 μM). No changes in either aPTT or PT relative to baseline were observed for the IgG4P isotype control in cynomolgus monkey plasma up to the highest antibody concentration tested (1.2 μM).

TABLE 15
Summary of aPTT (9 nM to 1.2 μM) and PT (600 nM
and 1.2 μM) in Cynomolgus Monkey Donor Plasma

| | Change in aPTT^{a} | | Change in PT^{a} | |
|---|---|---|---|---|
| Antibody | | IgG4^{P} | | IgG4^{P} |
| Concentration | | Isotype | | Isotype |
| (nM) | REGN7508 | Control | REGN7508 | Control |
| 9 | 1.05 | NT | NT | NT |
| 19 | 2.36 | 1.00 | | |
| 38 | 2.54 | 0.98 | | |
| 75 | 2.69 | 0.99 | | |
| 150 | 2.80 | 0.96 | | |
| 300 | 2.75 | 0.98 | | |
| 600 | 2.77 | 0.95 | 1.04 | 1.00 |
| 1200 | 2.83 | 0.95 | 1.04 | 1.01 |

^{a}Relative to baseline (ie, no antibody);
NT, Not tested

TABLE 16
Summary of aPTT in Cynomolgus Monkey
Donor Plasma (4 nM to 31 nM)

| | |
|---|---|
| Antibody | Change in aPTT^{a} |

| Concentration (nM) | REGN7508 | IgG4^{P} Isotype Control |
|---|---|---|
| 4 | 1.17 | NT |
| 8 | 1.25 | |
| 12 | 1.60 | |
| 16 | 2.15 | |
| 20 | 2.74 | |
| 24 | 2.78 | |
| 28 | 2.83 | |
| 31 | 2.85 | |

^{a}Relative to baseline (ie, no antibody);
NT, Not tested

### 9.2.2 Effect of REGN7508 in TGAs With Human or Cynomolgus Monkey Donor

### Plasma

TGAs with Human Plasma

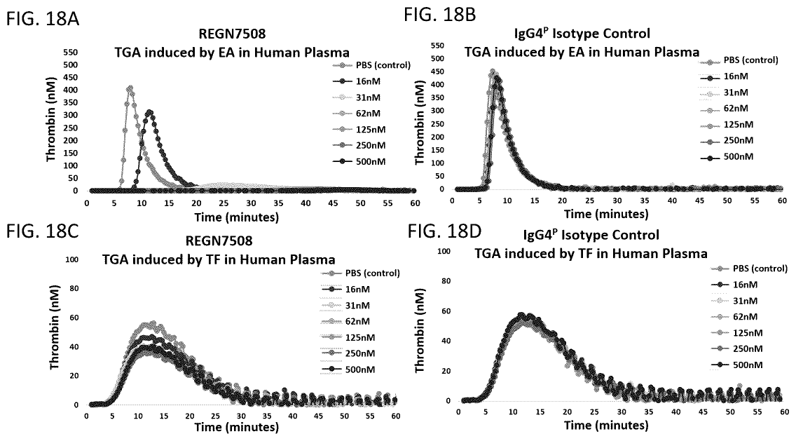

When thrombin generation was induced by EA via the intrinsic pathway in human plasma, REGN7508 increased the lag time for thrombin generation up to 6.6-fold relative to baseline (ie, no antibody), reduced peak thrombin levels down to 1% of baseline, and reduced endogenous thrombin potential down to 2% of baseline. REGN7508 exerted these effects in a concentration-dependent manner with maximal effects achieved at concentrations of ≥125 nM (FIG. 18A and Table 17). No increases in lag time for thrombin generation and only subtle decreases in peak thrombin levels and endogenous thrombin potential relative to baseline were observed with the IgG4P isotype control up to the highest antibody concentration tested (500 nM); these effects were not concentration-dependent (FIG. 18B and Table 17).

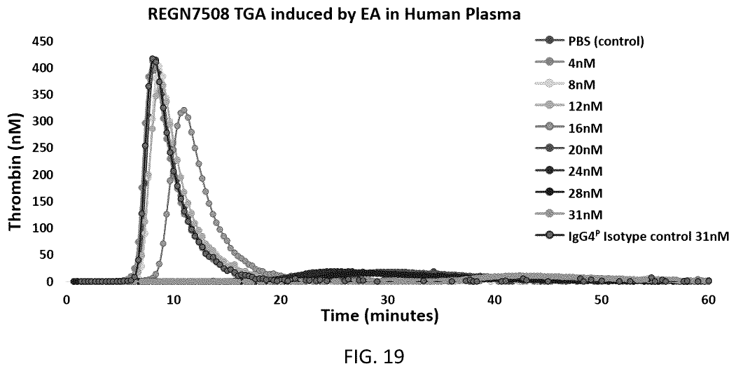

A second set of experiments was performed with REGN7508 concentrations ranging from 4 nM to 31 nM when tested in smaller increments, to obtain greater resolution of the thrombin peaks at concentrations less than 16 nM, however a gradual dose response was not observed. A steep change in PD effects followed by a plateau was observed at REGN7508 concentrations greater than 16 nM. Moreover, similar concentration-dependent effects on EA-induced thrombin generation were observed under otherwise identical assay conditions (FIG. 19 and Table 18).

When thrombin generation was induced by TF via the extrinsic pathway in human plasma, REGN7508 partially reduced peak thrombin levels and endogenous thrombin potential to 66% and 68% of baseline, respectively. REGN7508 exerted these effects in a concentration-dependent manner with maximal effects achieved at concentrations of ≥125 nM. No concentration-dependent increases in lag time for thrombin generation were observed with REGN7508 up to the highest antibody concentration tested (500 nM) (FIG. 18C and Table 19). No increases in lag time for thrombin generation or decreases in peak thrombin or endogenous thrombin potential were observed with the IgG4P isotype control up to the highest antibody concentration tested (500 nM) (FIG. 18D and Table 19).

TABLE 17
Summary of Parameters Measuring Intrinsic Pathway
TGA in Human Donor Plasma (16 nM to 500 nM)

| | Change in Thrombin Generation in Human Donor Plasma (TGA w/EA)^{a} | | | | | |
|---|---|---|---|---|---|---|
| | REGN7508 | | | IgG4^{P} Isotype Control | | |
| | Fold | | | Fold | | |
| Concentration | Change in | % Peak | % | Change in | % Peak | % |
| (nM) | Lag Time | Thrombin | ETP | Lag Time | Thrombin | ETP |
| 16 | 1.4 | 78 | 95 | 1.1 | 100 | 95 |
| 31 | 3.0 | 6 | 23 | 1.1 | 98 | 94 |
| 62 | 5.4 | 2 | 7 | 1.1 | 99 | 95 |
| 125 | 6.6 | 1 | 2 | 1.0 | 98 | 93 |
| 250 | 5.9 | 1 | 5 | 1.1 | 96 | 90 |
| 500 | 6.5 | 1 | 3 | 1.1 | 96 | 89 |

^{a}Changes in lag time, peak thrombin (%), and endogenous thrombin potential (% ETP) are relative to a no-antibody control (ie, PBS).

TABLE 18
Summary of Parameters Measuring Intrinsic Pathway
TGA in Human Donor Plasma (4 nM to 31 nM)

| | Change in Thrombin Generation in Human Donor Plasma (TGA w/EA) ^{a} | | | | | |
|---|---|---|---|---|---|---|
| | REGN7508 | | | IgG4^{P} Isotype Control | | |
| | Fold | | | Fold | | |
| Antibody | Change | | | Change | | |
| Concentration | in Lag | % Peak | % | in Lag | % Peak | % |
| (nM) | Time | Thrombin | ETP | Time | Thrombin | ETP |
| 4 | 1.0 | 100 | 104 | NT | | |
| 8 | 1.1 | 98 | 103 | | | |
| 12 | 1.1 | 92 | 102 | | | |
| 16 | 1.3 | 81 | 97 | | | |
| 20 | 3.7 | 5 | 20 | | | |
| 24 | 2.9 | 5 | 21 | | | |
| 28 | 3.3 | 4 | 18 | | | |
| 31 | 5.5 | 3 | 12 | 1.0 | 102 | 101 |

^{a} Changes in lag time, peak thrombin (%), and endogenous thrombin potential (% ETP) are relative to a no-antibody control (ie, PBS). NT: Not tested

TABLE 19
Summary of Parameters Measuring Extrinsic Pathway
TGA in Human Donor Plasma (16 nM to 500 nM)

| | Change in Thrombin Generation in Human Donor Plasma (TGA w/TF) ^{a} | | | | | |
|---|---|---|---|---|---|---|
| | REGN7508 | | | IgG4^{P} Isotype Control | | |
| | Fold | | | Fold | | |
| | Change | | | Change | | |
| Concentration | in Lag | % Peak | % | in Lag | % Peak | % |
| (nM) | Time | Thrombin | ETP | Time | Thrombin | ETP |
| 16 | 0.9 | 84 | 89 | 1.0 | 106 | 104 |
| 31 | 0.9 | 73 | 76 | 1.0 | 100 | 101 |
| 62 | 0.9 | 73 | 76 | 1.0 | 108 | 102 |
| 125 | 0.9 | 67 | 70 | 1.0 | 102 | 97 |
| 250 | 1.0 | 66 | 68 | 1.0 | 99 | 97 |
| 500 | 1.0 | 72 | 72 | 1.1 | 108 | 106 |

^{a} Changes in lag time, peak thrombin (%), and endogenous thrombin potential (% ETP) are relative to a no-antibody control (ie, PBS).

TGAs with Cynomolgus Monkey Plasma

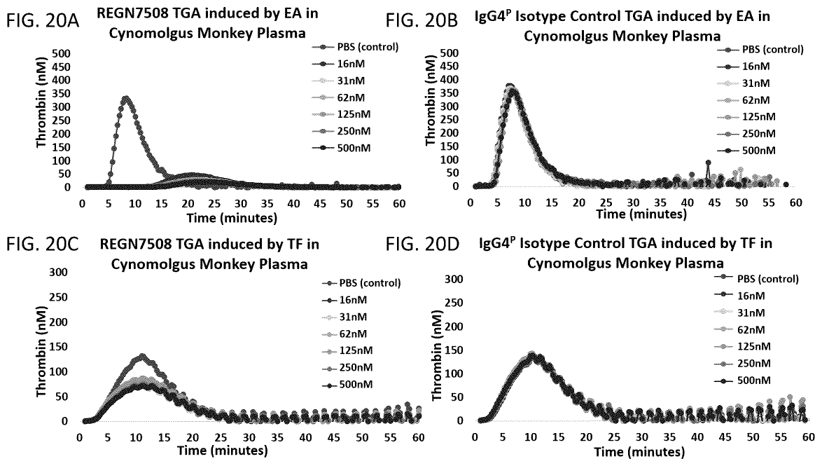

When thrombin generation was induced by EA via the intrinsic pathway in cynomolgus monkey plasma, REGN7508 increased the lag time for thrombin generation up to 2.8-fold relative to baseline (ie, no antibody), reduced peak thrombin levels down to 6% of baseline, and reduced endogenous thrombin potential down to 15% of baseline. REGN7508 exerted these effects in a concentration-dependent manner with maximal effects achieved at concentrations of ≥500 nM (FIG. 20A and Table 20). No increases in lag time for thrombin generation or decreases in peak thrombin or endogenous thrombin potential were observed with the IgG4P isotype control up to the maximum antibody concentration tested (500 nM) (FIG. 20B and Table 20).

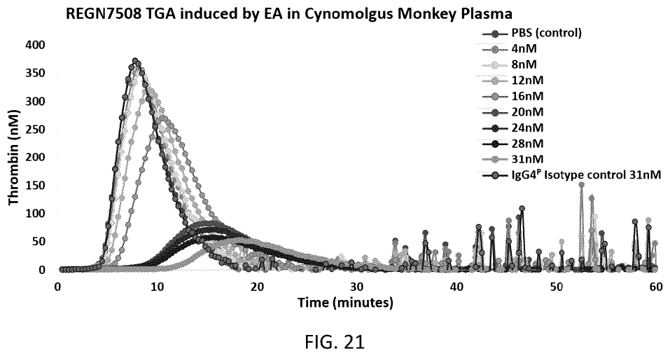

A second set of experiments was performed with REGN7508 concentrations ranging from 4 nM to 31 nM when tested in smaller increments, to obtain greater resolution of the thrombin curves at concentrations less than 16 nM, however a gradual dose response was not observed as it was a steep effect once target saturation was achieved. Moreover, similar concentration-dependent effects on EA-induced thrombin generation were observed under otherwise identical assay conditions (FIG. 21 and Table 21).

When thrombin generation was induced by TF via the extrinsic pathway in cynomolgus monkey plasma, REGN7508 reduced peak thrombin levels down to 55% of baseline and reduced endogenous thrombin potential down to 65% of baseline. REGN7508 exerted these effects in a concentration-dependent manner with maximal effects achieved at concentrations of ≥125 nM (FIG. 20C and Table 22). No concentration-dependent increases in lag time for thrombin generation were observed with REGN7508 up to the maximum antibody concentration tested (500 nM). No increases in lag time for thrombin generation or decreases in peak thrombin or endogenous thrombin potential were observed or the IgG4P isotyp control up to the maximum antibody concentration tested (500 nM) (FIG. 20D and Table 22).

TABLE 20
Summary of Parameters Measuring Intrinsic Pathway TGA
in Cynomolgus Monkey Donor Plasma (16 nM to 500 nM)

| | Change in Thrombin Generation in Cynomolgus Monkey Donor Plasma (TGA w/EA)^{a} | | | | | |
|---|---|---|---|---|---|---|
| | REGN7508 | | | IgG4^{P} Isotype Control | | |
| | Fold | | | Fold | | |
| Concentration | Change in | % Peak | % | Change in | % Peak | % |
| (nM) | Lag Time | Thrombin | ETP | Lag Time | Thrombin | ETP |
| 16 | 2.6 | 14 | 27 | 1.0 | 106 | 104 |
| 31 | 2.6 | 8 | 18 | 1.0 | 103 | 101 |
| 62 | 2.8 | 8 | 17 | 1.0 | 101 | 102 |
| 125 | 2.6 | 8 | 18 | 1.1 | 102 | 99 |
| 250 | 2.7 | 7 | 15 | 1.0 | 100 | 105 |
| 500 | 2.8 | 6 | 15 | 1.0 | 100 | 102 |

^{a}Changes in lag time, peak thrombin (%), and endogenous thrombin potential (% ETP) are relative to a no-antibody control (ie, PBS).

TABLE 21
Summary of Parameters Measuring Intrinsic Pathway TGA
in Cynomolgus Monkey Donor Plasma (4 nM to 31 nm)

| | Change in Thrombin Generation in Cynomolgus Monkey Donor Plasma (TGA w/EA) ^{a} | | | | | |
|---|---|---|---|---|---|---|
| | REGN7508 | | | IgG4^{P} Isotype Control | | |
| | Fold | | | Fold | | |
| Antibody | Change | | | Change | | |
| Concentration | in Lag | % Peak | % | in Lag | % Peak | % |
| (nM) | Time | Thrombin | ETP | Time | Thrombin | ETP |
| 4 | 1.0 | 97 | 105 | NT | | |
| 8 | 1.0 | 95 | 104 | | | |
| 12 | 1.1 | 87 | 103 | | | |
| 16 | 1.4 | 73 | 95 | | | |
| 20 | 1.9 | 23 | 42 | | | |
| 24 | 1.9 | 20 | 38 | | | |
| 28 | 1.9 | 16 | 32 | | | |
| 31 | 2.5 | 14 | 29 | 1.0 | 100 | 97 |

^{a} Changes in lag time, peak thrombin (%), and endogenous thrombin potential (% ETP) are relative to a no-antibody control (ie, PBS). NT: Not tested.

TABLE 22
Summary of Parameters Measuring Extrinsic Pathway
in Cynomolgus Monkey Donor Plasma (16 nM to 500 nM)

| | Change in Thrombin Generation in Cynomolgus Monkey Donor Plasma (TGA w/TF) ^{a} | | | | | |
|---|---|---|---|---|---|---|
| | REGN7508 | | | IgG4^{P} Isotype Control | | |
| | Fold | | | Fold | | |
| Concentration | Change in | % Peak | % | Change in | % Peak | % |
| (nM) | Lag Time | Thrombin | ETP | Lag Time | Thrombin | ETP |
| 16 | 0.8 | 60 | 70 | 0.9 | 96 | 98 |
| 31 | 0.8 | 55 | 65 | 0.9 | 98 | 97 |
| 62 | 0.8 | 57 | 68 | 0.9 | 99 | 98 |
| 125 | 0.8 | 66 | 78 | 0.9 | 102 | 105 |
| 250 | 0.8 | 57 | 69 | 0.9 | 96 | 97 |
| 500 | 0.8 | 55 | 66 | 0.9 | 99 | 100 |

^{a} Changes in lag time, peak thrombin (%), and endogenous thrombin potential (% ETP) are relative to a no-antibody control (ie, PBS).

### CONCLUSIONS

REGN7508 mediated complete blockade of the intrinsic coagulation pathway in human and cynomolgus monkey plasma in a concentration-dependent manner. REGN7508 also mediated concentration-dependent partial blockade of the extrinsic coagulation pathway in human and cynomolgus monkey plasma, albeit to a much lesser degree than effects on the intrinsic pathway.

### Example 10: Evaluation of REGN7508-FXI Immune Complexes for Binding to C1q

Circulating immune complexes (CIC) are formed by multimerization of antibody with soluble antigen. Deposition of CIC within tissues and consequent inflammatory responses can lead to tissue damage at the site of deposition. Large immune complexes can also activate complement component C1q in serum (Rojko, 2014).

REGN7508 is unlikely to form immune complexes capable of binding C1q because it contains a hinge-stabilized, IgG4-derived heavy chain fragment crystallizable (Fc) constant domain (termed IgG4P), and IgG4 does not bind as well as IgG1 to C1q (Patel, 2015). Nevertheless, an enzyme immunosorbent assay (EIA) was performed to evaluate the potential for binding of REGN7508-FXI and REGN7508-FXIa complexes to C1q.

REGN7508-FXI and REGN7508-FXIa complexes did not demonstrate detectable binding to C1q, consistent with the minimal effector function activity of IgG4-based antibodies.

### Example 11: Assessment of Pharmacokinetics and Toxicokinetics of REGN7508 in Cynomolgus Monkeys

Characterization of REGN7508-mediated blockade of the coagulation pathway in cynomolgus monkeys was assessed as part of single-dose pharmacokinetic (PK) studies. The results of these studies show that REGN7508 mediated inhibition of the intrinsic coagulation pathway with minimal effects on the extrinsic coagulation pathway. The results of these studies further show no tolerability issues after SC and IV dosing of REGN7508.

INFORMAL SEQUENCE LISTING

| SEQ ID NO: | Sequence |
|---|---|
| 1 | SEQ ID NO: 1. |
| | CAGGTGCAGCTGGTGGAGTCTGGGGGAGGCTTGGTCAAGCCTGGAGGGTCCCTGAGACTC |
| | TCCTGTGCAGCCTCTGGATTCACCTTCAGTGACTACTCCATGAACTGGATCCGCCAGGCT |
| | CCAGGGAAGGGGCTGGAGTGGATCTCATACATTAGTTTTAGTGGTAATAGCATATACTAC |
| | GCAGACTCTGTGAAGGGCCGATTCACCATCTCCAGGGACAACGCCAAGAACTCCCTATAT |
| | CTGCAAATGAACAGCCTGAGAGTCGAGGACACGGCCATATATTACTGTACGAGCCGGGA |
| | CTGGGGATATGCTTTTGATATCTGGGGCCAAGGGACAATGGTCACCGTCTCTTCA |
| 2 | SEQ ID NO: 2. |
| | QVQLVESGGGLVKPGGSLRLSCAASGFTFSDYSMNWIRQAPGKGLEWISYISFSGNSIY |
| | YADSVKGRFTISRDNAKNSLYLQMNSLRVEDTAIYYCTSRDWGYAFDIWGQGTMVTVSS |
| 3 | SEQ ID NO: 3. |
| | GGA TTC ACC TTC AGT GAC TAC TCC |
| 4 | SEQ ID NO: 4. |
| | G F T F S D Y S |
| 5 | SEQ ID NO: 5. |
| | ATT AGT TTT AGT GGT AAT AGC ATA |
| 6 | SEQ ID NO: 6. |
| | I S F S G N S I |
| 7 | SEQ ID NO: 7. |
| | ACG AGC CGG GAC TGG GGA TAT GCT TTT GAT ATC |
| 8 | SEQ ID NO: 8. |
| | T S R D W G Y A F D I |
| 9 | SEQ ID NO: 9. |
| | GACATCCAGATGACCCAGTCTCCATCCTTCCTGTCTGCATCTTTAGGAGACAGAGTCACC |
| | ATCACTTGCCAGGCGAGTCAGGACATTAGCAATTATTTAAATTGGTATCAGGTGAAGCCA |
| | GGGAAAGCCCCTAAGCTCCTGATCTACGATGCATCCAATTTGGAAACAGGGGTCCCATCA |
| | AGGTTCAGTGGAAGTGGCTCTGGGACAGATTTTACTTTCACCATCAGCAGCCTGCAGCCT |
| | GAAGATATTGCAACATATTTCTGTCAACAATATGATAATCTCCCGTACATTTTTGGCCAG |
| | GGGACCAAGCTGGAGATCAAA |
| 10 | SEQ ID NO: 10. |
| | DIQMTQSPSFLSASLGDRVTITCQASQDISNYLNWYQVKPGKAPKLLIYDASNLETGVPS |
| | RFSGSGSGTDFTFTISSLQPEDIATYFCQQYDNLPYIFGQGTKLEIK |
| 11 | SEQ ID NO: 11. |
| | CAG GAC ATT AGC AAT TAT |
| 12 | SEQ ID NO: 12. |
| | Q D I S N Y |
| 13 | SEQ ID NO: 13. |
| | GAT GCA TCC |
| 14 | SEQ ID NO: 14. |
| | D A S |
| 15 | SEQ ID NO: 15. |
| | CAA CAA TAT GAT AAT CTC CCG TAC ATT |
| 16 | SEQ ID NO: 16. |
| | Q Q Y D N L P Y I |
| 17 | SEQ ID NO: 17. |
| | CAGGTGCAGCTGGTGGAGTCTGGGGGAGGCTTGGTCAAGCCTGGAGGGTCCCTGAGACTC |
| | TCCTGTGCAGCCTCTGGATTCACCTTCAGTGACTACTCCATGAACTGGATCCGCCAGGCT |
| | CCAGGGAAGGGGCTGGAGTGGATCTCATACATTAGTTTTAGTGGTAATAGCATATACTAC |
| | GCAGACTCTGTGAAGGGCCGATTCACCATCTCCAGGGACAACGCCAAGAACTCCCTATAT |
| | CTGCAAATGAACAGCCTGAGAGTCGAGGACACGGCCATATATTACTGTACGAGCCGGGAC |
| | TGGGGATATGCTTTTGATATCTGGGGCCAAGGGACAATGGTCACCGTCTCTTCAGCCTCC |
| | ACCAAGGGCCCATCGGTCTTCCCCCTGGCGCCCTGCTCCAGGAGCACCTCCGAGAGCACA |
| | GCCGCCCTGGGCTGCCTGGTCAAGGACTACTTCCCCGAACCGGTGACGGTGTCGTGGAAC |
| | TCAGGCGCCCTGACCAGCGGCGTGCACACCTTCCCGGCTGTCCTACAGTCCTCAGGACTC |
| | TACTCCCTCAGCAGCGTGGTGACCGTGCCCTCCAGCAGCTTGGGCACGAAGACCTACACC |
| | TGCAACGTAGATCACAAGCCCAGCAACACCAAGGTGGACAAGAGAGTTGAGTCCAAATAT |
| | GGTCCCCCATGCCCACCCTGCCCAGCACCTGAGTTCCTGGGGGGACCATCAGTCTTCCTG |
| | TTCCCCCCAAAACCCAAGGACACTCTCATGATCTCCCGGACCCCTGAGGTCACGTGCGTG |
| | GTGGTGGACGTGAGCCAGGAAGACCCCGAGGTCCAGTTCAACTGGTACGTGGATGGCGTG |
| | GAGGTGCATAATGCCAAGACAAAGCCGCGGGAGGAGCAGTTCAACAGCACGTACCGTGTG |
| | GTCAGCGTCCTCACCGTCCTGCACCAGGACTGGCTGAACGGCAAGGAGTACAAGTGCAAG |
| | GTCTCCAACAAAGGCCTCCCGTCCTCCATCGAGAAAACCATCTCCAAAGCCAAAGGGCAG |
| | CCCCGAGAGCCACAGGTGTACACCCTGCCCCCATCCCAGGAGGAGATGACCAAGAACCAG |
| | GTCAGCCTGACCTGCCTGGTCAAAGGCTTCTACCCCAGCGACATCGCCGTGGAGTGGGAG |
| | AGCAATGGGCAGCCGGAGAACAACTACAAGACCACGCCTCCCGTGCTGGACTCCGACGGC |
| | TCCTTCTTCCTCTACAGCAGGCTCACCGTGGACAAGAGCAGGTGGCAGGAGGGGAATGTC |
| | TTCTCATGCTCCGTGATGCATGAGGCTCTGCACAACCACTACACACAGAAGTCCCTCTCC |
| | CTGTCTCTGGGTAAATGA |
| 18 | SEQ ID NO: 18. |
| | QVQLVESGGGLVKPGGSLRLSCAASGFTFSDYSMNWIRQAPGKGLEWISYISFSGNSIYYADS |
| | VKGRFTISRDNAKNSLYLQMNSLRVEDTAIYYCTSRDWGYAFDIWGQGTMVTVSSASTKGPSV |
| | FPLAPCSRSTSESTAALGCLVKDYFPEPVTVSWNSGALTSGVHTFPAVLQSSGLYSLSSVVTV |
| | PSSSLGTKTYTCNVDHKPSNTKVDKRVESKYGPPCPPCPAPEFLGGPSVFLFPPKPKDTLMIS |
| | RTPEVTCVVVDVSQEDPEVQFNWYVDGVEVHNAKTKPREEQFNSTYRVVSVLTVLHQDWLNGK |
| | EYKCKVSNKGLPSSIEKTISKAKGQPREPQVYTLPPSQEEMTKNQVSLTCLVKGFYPSDIAVE |
| | WESNGQPENNYKTTPPVLDSDGSFFLYSRLTVDKSRWQEGNVFSCSVMHEALHNHYTQKSL |
| | SLSLGK* |
| 19 | SEQ ID NO: 19. |
| | GACATCCAGATGACCCAGTCTCCATCCTTCCTGTCTGCATCTTTAGGAGACAGAGTCACC |
| | ATCACTTGCCAGGCGAGTCAGGACATTAGCAATTATTTAAATTGGTATCAGGTGAAGCCA |
| | GGGAAAGCCCCTAAGCTCCTGATCTACGATGCATCCAATTTGGAAACAGGGGTCCCATCA |
| | AGGTTCAGTGGAAGTGGCTCTGGGACAGATTTTACTTTCACCATCAGCAGCCTGCAGCCT |
| | GAAGATATTGCAACATATTTCTGTCAACAATATGATAATCTCCCGTACATTTTTGGCCAG |
| | GGGACCAAGCTGGAGATCAAACGAACTGTGGCTGCACCATCTGTCTTCATCTTCCCGCCA |
| | TCTGATGAGCAGTTGAAATCTGGAACTGCCTCTGTTGTGTGCCTGCTGAATAACTTCTAT |
| | CCCAGAGAGGCCAAAGTACAGTGGAAGGTGGATAACGCCCTCCAATCGGGTAACTCCCAG |
| | GAGAGTGTCACAGAGCAGGACAGCAAGGACAGCACCTACAGCCTCAGCAGCACCCTGACG |
| | CTGAGCAAAGCAGACTACGAGAAACACAAAGTCTACGCCTGCGAAGTCACCCATCAGGGC |
| | CTGAGCTCGCCCGTCACAAAGAGCTTCAACAGGGGAGAGTGTTAG |
| 20 | SEQ ID NO: 20. |
| | DIQMTQSPSFLSASLGDRVTITCQASQDISNYLNWYQVKPGKAPKLLIYDASNLETGVPSR |
| | FSGSGSGTDFTFTISSLQPEDIATYFCQQYDNLPYIFGQGTKLEIKRTVAAPSVFIFPPSD |
| | EQLKSGTASVVCLLNNFYPREAKVQWKVDNALQSGNSQESVTEQDSKDSTYSLSSTLTLSK |
| | ADYEKHKVYACEVTHQGLSSPVTKSFNRGEC* |
| 21 | SEQ ID NO: 21. |
| | CAGGTGCAGCTGGTGGAGTCTGGGGGAGGCGTGGTCCAGCCTGGGAGGTCCCTGAGACT |
| | CTCCTGTGCAGCGTCTGGATTCACCTTCAGTAACTATGAAATGCACTGGGTCCGCCAGA |
| | CTCCAGGCAAGGGGCTGGAGTGGGTGGCACTTATTTGGTATGATGGAAGTAATAAATAT |
| | TCTGGAGATTCCGTGAAGGGCCGATTCACCATTTCCAGAGACAATTCCAAGAGTACGCT |
| | AGTTCTGCAAATGAACAGCCTGACAGCCGAGGACACGGCTGTTTATTACTGCACCGGTA |
| | TAACTGGAACTGAGGATGCTTTTGATGTCTGGGGCCAAGGGACAATGGTCACCGTCTCT |
| | TCA |
| 22 | SEQ ID NO: 22. |
| | QVQLVESGGGVVQPGRSLRLSCAASGFTFSNYEMHWVRQTPGKGLEWVALIWYDGSNKY |
| | SGDSVKGRFTISRDNSKSTLYLQMNSLTAEDTAVYYCTGITGTEDAFDVWGQGTMVTVS |
| | S |
| 23 | SEQ ID NO: 23. |
| | GGA TTC ACC TTC AGT AAC TAT GAA |
| 24 | SEQ ID NO: 24. |
| | G F T F S N Y E |
| 25 | SEQ ID NO: 25. |
| | ATT TGG TAT GAT GGA AGT AAT AAA |
| 26 | SEQ ID NO: 26. |
| | I W Y D G S N K |
| 27 | SEQ ID NO: 27. |
| | ACC GGT ATA ACT GGA ACT GAG GAT GCT TTT GAT GTC |
| 28 | SEQ ID NO: 28. |
| | T G I T G T E D A F D V |
| 29 | SEQ ID NO: 29. |
| | GACATCCAGATGACCCAGTCTCCATCCTCCCTGTCTGCATCTGTAGGAGACAGAGTCACC |
| | ATCACTTGCCGGGCAAGTCAGAGCATTAGCAGCTATTTAAATTGGTATCAGCAGAAACCA |
| | GGGAAAGCCCCTAAGCTCCTGATCTATGCTGCATCCAGTTTGCAAAGTGGGGTCCCGTCA |
| | AGGTTCAGTGGCAGTGGATCTGGGACAGATTTCACTCTCACCATCAGCAGTCTGCAACCT |
| | GAAGATTTTGCAACTTACTACTGTCAACAGAGTTACAGTACCCCTCCGATCACCTTCGGC |
| | CAAGGGACACGACTGGAGATTAAA |
| 30 | SEQ ID NO: 30. |
| | DIQMTQSPSSLSASVGDRVTITCRASQSISSYLNWYQQKPGKAPKLLIYAASSLQSGVPSR |
| | FSGSGSGTDFTLTISSLQPEDFATYYCQQSYSTPPITFGQGTRLEIK |
| 31 | SEQ ID NO: 31. |
| | CAG AGC ATT AGC AGC TAT |
| 32 | SEQ ID NO: 32. |
| | Q S I S S Y |
| 33 | SEQ ID NO: 33. |
| | GCT GCA TCC |
| 34 | SEQ ID NO: 34. |
| | A A S |
| 35 | SEQ ID NO: 35. |
| | CAA CAG AGT TAC AGT ACC CCT CCG ATC ACC |
| 36 | SEQ ID NO: 36. |
| | Q Q S Y S T P P I T |
| 37 | SEQ ID NO: 37. |
| | CAGGTGCAGCTGGTGGAGTCTGGGGGAGGCGTGGTCCAGCCTGGGAGGTCCCTGAGACTC |
| | TCCTGTGCAGCGTCTGGATTCACCTTCAGTAACTATGAAATGCACTGGGTCCGCCAGACT |
| | CCAGGCAAGGGGCTGGAGTGGGTGGCACTTATTTGGTATGATGGAAGTAATAAATATTCT |
| | GGAGATTCCGTGAAGGGCCGATTCACCATTTCCAGAGACAATTCCAAGAGTACGCTGTAT |
| | CTGCAAATGAACAGCCTGACAGCCGAGGACACGGCTGTTTATTACTGCACCGGTATAACT |
| | GGAACTGAGGATGCTTTTGATGTCTGGGGCCAAGGGACAATGGTCACCGTCTCTTCAGCC |
| | TCCACCAAGGGCCCATCGGTCTTCCCCCTGGCGCCCTGCTCCAGGAGCACCTCCGAGAGC |
| | ACAGCCGCCCTGGGCTGCCTGGTCAAGGACTACTTCCCCGAACCGGTGACGGTGTCGTGG |
| | AACTCAGGCGCCCTGACCAGCGGCGTGCACACCTTCCCGGCTGTCCTACAGTCCTCAGGA |
| | CTCTACTCCCTCAGCAGCGTGGTGACCGTGCCCTCCAGCAGCTTGGGCACGAAGACCTAC |
| | ACCTGCAACGTAGATCACAAGCCCAGCAACACCAAGGTGGACAAGAGAGTTGAGTCCAAA |
| | TATGGTCCCCCATGCCCACCCTGCCCAGCACCTGAGTTCCTGGGGGGACCATCAGTCTTC |
| | CTGTTCCCCCCAAAACCCAAGGACACTCTCATGATCTCCCGGACCCCTGAGGTCACGGCG |
| | TGGTGGTGGACGTGAGCCAGGAAGACCCCGAGGTCCAGTTCAACTGGTACGTGGATTGGC |
| | GTGGAGGTGCATAATGCCAAGACAAAGCCGCGGGAGGAGCAGTTCAACAGCACGTACCGT |
| | GTGGTCAGCGTCCTCACCGTCCTGCACCAGGACTGGCTGAACGGCAAGGAGTACAAGTGC |
| | AAGGTCTCCAACAAAGGCCTCCCGTCCTCCATCGAGAAAACCATCTCCAAAGCCAAAGGG |
| | CAGCCCCGAGAGCCACAGGTGTACACCCTGCCCCCATCCCAGGAGGAGATGACCAAGAAC |
| | CAGGTCAGCCTGACCTGCCTGGTCAAAGGCTTCTACCCCAGCGACATCGCCGTGGAGTGG |
| | GAGAGCAATGGGCAGCCGGAGAACAACTACAAGACCACGCCTCCCGTGCTGGACTCCGAC |
| | GGCTCCTTCTTCCTCTACAGCAGGCTCACCGTGGACAAGAGCAGGTGGCAGGAGGGGAAT |
| | GTCTTCTCATGCTCCGTGATGCATGAGGCTCTGCACAACCACTACACACAGAAGTCCCTC |
| | TCCCTGTCTCTGGGTAAATGA |
| 38 | SEQ ID NO: 38. |
| | QVQLVESGGGVVQPGRSLRLSCAASGFTFSNYEMHWVRQTPGKGLEWVALIWYDGSNKYS |
| | GDSVKGRFTISRDNSKSTLYLQMNSLTAEDTAVYYCTGITGTEDAFDVWGQGTMVTVSSA |
| | STKGPSVFPLAPCSRSTSESTAALGCLVKDYFPEPVTVSWNSGALTSGVHTFPAVLQSSG |
| | LYSLSSVVTVPSSSLGTKTYTCNVDHKPSNTKVDKRVESKYGPPCPPCPAPEFLGGPSVF |
| | LFPPKPKDTLMISRTPEVTCVVVDVSQEDPEVQFNWYVDGVEVHNAKTKPREEQFNSTYR |
| | VVSVLTVLHQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDKSRWQEGN |
| | VFSCSVMHEALHNHYTQKSLSLSLGK* |
| 39 | SEQ ID NO: 39. |
| | GACATCCAGATGACCCAGTCTCCATCCTCCCTGTCTGCATCTGTAGGAGACAGAGTCACC |
| | ATCACTTGCCGGGCAAGTCAGAGCATTAGCAGCTATTTAAATTGGTATCAGCAGAAACCA |
| | GGGAAAGCCCCTAAGCTCCTGATCTATGCTGCATCCAGTTTGCAAAGTGGGGTCCCGTCA |
| | AGGTTCAGTGGCAGTGGATCTGGGACAGATTTCACTCTCACCATCAGCAGTCTGCAACCT |
| | GAAGATTTTGCAACTTACTACTGTCAACAGAGTTACAGTACCCCTCCGATCACCTTCGGC |
| | CAAGGGACACGACTGGAGATTAAACGAACTGTGGCTGCACCATCTGTCTTCATCTTCCCG |
| | CCATCTGATGAGCAGTTGAAATCTGGAACTGCCTCTGTTGTGTGCCTGCTGAATAACTTC |
| | TATCCCAGAGAGGCCAAAGTACAGTGGAAGGTGGATAACGCCCTCCAATCGGGTAACTCC |
| | CAGGAGAGTGTCACAGAGCAGGACAGCAAGGACAGCACCTACAGCCTCAGCAGCACCCTG |
| | ACGCTGAGCAAAGCAGACTACGAGAAACACAAAGTCTACGCCTGCGAAGTCACCCATCAG |
| | GGCCTGAGCTCGCCCGTCACAAAGAGCTTCAACAGGGGAGAGTGTTAG |
| 40 | SEQ ID NO: 40. |
| | DIQMTQSPSSLSASVGDRVTITCRASQSISSYLNWYQQKPGKAPKLLIYAASSLQSGVPS |
| | RFSGSGSGTDFTLTISSLQPEDFATYYCQQSYSTPPITFGQGTRLEIKRTVAAPSVFIFP |
| | PSDEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC* |
| 41 | MIFLYQVVHFILFTSVSGECVTQLLKDTCFEGGDITTVFTPSAKYCQVVCTYHPRCLLFT |
| | FTAESPSEDPTRWFTCVLKDSVTETLPRVNRTAAISGYSFKQCSHQISACNKDIYVDLDM |
| | KGINYNSSVAKSAQECQERCTDDVHCHFFTYATRQFPSLEHRNICLLKHTQTGTPTRITK |
| | LDKVVSGFSLKSCALSNLACIRDIFPNTVFADSNIDSVMAPDAFVCGRICTHHPGCLFFT |
| | FFSQEWPKESQRNLCLLKTSESGLPSTRIKKSKALSGFSLQSCRHSIPVFCHSSFYHDTD |
| | FLGEELDIVAAKSHEACQKLCTNAVRCQFFTYTPAQASCNEGKGKCYLKLSSNGSPTKIL |
| | HGRGGISGYTLRLCKMDNECTTKIKPRIVGGTASVRGEWPWQVTLHTTSPTQRHLCGGSI |
| | IGNQWILTAAHCFYGVESPKILRVYSGILNQSEIKEDTSFFGVQEIIIHDQYKMAESGYD |
| | IALLKLETTVNYTDSQRPICLPSKGDRNVIYTDCWVTGWGYRKLRDKIQNTLQKAKIPLV |
| | TNEECQKRYRGHKITHKMICAGYREGGKDACKGDSGGPLSCKHNEVWHLVGITSWGEGCA |
| | QRERPGVYTNVVEYVDWILEKTQAV |
| 42 | ECVTQLLKDTCFEGGDITTVFTPSAKYCQVVCTYHPRCLLFTFTAESPSEDPTRWFTCVL |
| | KDSVTETLPRVNRTAAISGYSFKQCSHQISACNKDIYVDLDMKGINYNSSVAKSAQECQE |
| | RCTDDVHCHFFTYATRQFPSLEHRNICLLKHTQTGTPTRITKLDKVVSGFSLKSCALSNL |
| | ACIRDIFPNTVFADSNIDSVMAPDAFVCGRICTHHPGCLFFTFFSQEWPKESQRNLCLLK |
| | TSESGLPSTRIKKSKALSGFSLQSCRHSIPVFCHSSFYHDTDFLGEELDIVAAKSHEACQ |
| | KLCTNAVRCQFFTYTPAQASCNEGKGKCYLKLSSNGSPTKILHGRGGISGYTLRLCKMDN |
| | ECTTKIKPRIVGGTASVRGEWPWQVTLHTTSPTQRHLCGGSIIGNQWILTAAHCFYGVES |
| | PKILRVYSGILNQSEIKEDTSFFGVQEIIIHDQYKMAESGYDIALLKLETTVNYTDSQRP |
| | ICLPSKGDRNVIYTDCWVTGWGYRKLRDKIQNTLQKAKIPLVTNEECQKRYRGHKITHKM |
| | ICAGYREGGKDACKGDSGGPLSCKHNEVWHLVGITSWGEGCAQRERPGVYTNVVEYVDWI |
| | LEKTQAVEQKLISEEDLGGEQKLISEEDLHHHHHH |
| 43 | GCLTQLYENAFFRGGDVASMYTPNAQYCQMRCTFHPRCLLFSFLPASSINDMEKRFGCFL |
| | KDSVTGTLPKVHRTGAVSGHSLKQCHQISACNKDIYVDLDMKGINYNSSVAKSAQECQER |
| | CTDDVHCHFFTYATRQFPSLEHRNICLLKHTQTGTPTRITKLDKVVSGFSLKSCALSNLA |
| | CIRDIFPNTVFADSNIDSVMAPDAFVCGRICTHHPGCLFFTFFSQEWPKESQRNLCLLKT |
| | SESGLPSTRIKKSKALSGFSLQSCRHSIPVFCHSSFYHDTDFLGEELDIVAAKSHEACQK |
| | LCTNAVRCQFFTYTPAQASCNEGKGKCYLKLSSNGSPTKILHGRGGISGYTLRLCKMDNE |
| | CTTKIKPRIVGGTASVRGEWPWQVTLHTTSPTQRHLCGGSIIGNQWILTAAHCFYGVESP |
| | KILRVYSGILNQSEIKEDTSFFGVQEIIIHDQYKMAESGYDIALLKLETTVNYTDSQRPI |
| | CLPSKGDRNVIYTDCWVTGWGYRKLRDKIQNTLQKAKIPLVTNEECQKRYRGHKITHKMI |
| | CAGYREGGKDACKGDSGGPLSCKHNEVWHLVGITSWGEGCAQRERPGVYTNVVEYVDWIL |
| | EKTQAVEQKLISEEDLGGEQKLISEEDLHHHHHH |
| 44 | ECVTQLLKDTCFEGGDITTVFTPSAKYCQVVCTYHPRCLLFTFTAESPSEDPTRWFTCVL |
| | KDSVTETLPRVNRTAAISGYSFKQCSHQISCHRDIYKGVDMRGVNFNVSKVSSVEECQKR |
| | CTNNIRCQFFSYATQTFHKAEYRNNCLLKYSPGGTPTAIKVLSNVESGFSLKPCLSNLAC |
| | IRDIFPNTVFADSNIDSVMAPDAFVCGRICTHHPGCLFFTFFSQEWPKESQRNLCLLKTS |
| | ESGLPSTRIKKSKALSGFSLQSCRHSIPVFCHSSFYHDTDFLGEELDIVAAKSHEACQKL |
| | CTNAVRCQFFTYTPAQASCNEGKGKCYLKLSSNGSPTKILHGRGGISGYTLRLCKMDNEC |
| | TTKIKPRIVGGTASVRGEWPWQVTLHTTSPTQRHLCGGSIIGNQWILTAAHCFYGVESPK |
| | ILRVYSGILNQSEIKEDTSFFGVQEIIIHDQYKMAESGYDIALLKLETTVNYTDSQRPIC |
| | LPSKGDRNVIYTDCWVTGWGYRKLRDKIQNTLQKAKIPLVTNEECQKRYRGHKITHKMIC |
| | AGYREGGKDACKGDSGGPLSCKHNEVWHLVGITSWGEGCAQRERPGVYTNVVEYVDWILE |
| | KTQAVEQKLISEEDLGGEQKLISEEDLHHHHHH |
| 45 | ECVTQLLKDTCFEGGDITTVFTPSAKYCQVVCTYHPRCLLFTFTAESPSEDPTRWFTCVL |
| | KDSVTETLPRVNRTAAISGYSFKQCSHQISACNKDIYVDLDMKGINYNSSVAKSAQECQE |
| | RCTDDVHCHFFTYATRQFPSLEHRNICLLKHTQTGTPTRITKLDKVVSGFSLKSCALSNL |
| | CHMNIFQHLAFSDVDVARVLTPDAFVCRTICTYHPNCLFFTFYTNVWKIESQRNVCLLKT |
| | SESGTPSSSTPQENTISGYSLLTCHSIPVFCHSSFYHDTDFLGEELDIVAAKSHEACQKL |
| | CTNAVRCQFFTYTPAQASCNEGKGKCYLKLSSNGSPTKILHGRGGISGYTLRLCKMDNEC |
| | TTKIKPRIVGGTASVRGEWPWQVTLHTTSPTQRHLCGGSIIGNQWILTAAHCFYGVESPK |
| | ILRVYSGILNQSEIKEDTSFFGVQEIIIHDQYKMAESGYDIALLKLETTVNYTDSQRPIC |
| | LPSKGDRNVIYTDCWVTGWGYRKLRDKIQNTLQKAKIPLVTNEECQKRYRGHKITHKMIC |
| | AGYREGGKDACKGDSGGPLSCKHNEVWHLVGITSWGEGCAQRERPGVYTNVVEYVDWILE |
| | KTQAVEQKLISEEDLGGEQKLISEEDLHHHHHH |
| 46 | ECVTQLLKDTCFEGGDITTVFTPSAKYCQVVCTYHPRCLLFTFTAESPSEDPTRWFTCVL |
| | KDSVTETLPRVNRTAAISGYSFKQCSHQISACNKDIYVDLDMKGINYNSSVAKSAQECQE |
| | RCTDDVHCHFFTYATRQFPSLEHRNICLLKHTQTGTPTRITKLDKVVSGFSLKSCALSNL |
| | ACIRDIFPNTVFADSNIDSVMAPDAFVCGRICTHHPGCLFFTFFSQEWPKESQRNLCLLK |
| | TSESGLPSTRIKKSKALSGFSLQSCRHSIPVCHSKIYPGVDFGGEELNVTFVKGVNVCQE |
| | TCTKMIRCQFFTYSLLPEDCKEEKGKCFLRLSMDGSPTRIAYGTQGSSGYSLRLCMDNEC |
| | TTKIKPRIVGGTASVRGEWPWQVTLHTTSPTQRHLCGGSIIGNQWILTAAHCFYGVESPK |
| | ILRVYSGILNQSEIKEDTSFFGVQEIIIHDQYKMAESGYDIALLKLETTVNYTDSQRPIC |
| | LPSKGDRNVIYTDCWVTGWGYRKLRDKIQNTLQKAKIPLVTNEECQKRYRGHKITHKMIC |
| | AGYREGGKDACKGDSGGPLSCKHNEVWHLVGITSWGEGCAQRERPGVYTNVVEYVDWILE |
| | KTQAVEQKLISEEDLGGEQKLISEEDLHHHHHH |
| 47 | GCLTQLYENAFFRGGDVASMYTPNAQYCQMRCTFHPRCLLFSFLPASSINDMEKRFGCFL |
| | KDSVTGTLPKVHRTGAVSGHSLKQCGHQISACHRDIYKGVDMRGVNFNVSKVSSVEECQK |
| | RCTNNIRCQFFSYATQTFHKAEYRNNCLLKYSPGGTPTAIKVLSNVESGFSLKPCALSEI |
| | GCHMNIFQHLAFSDVDVARVLTPDAFVCRTICTYHPNCLFFTFYTNVWKIESQRNVCLLK |
| | TSESGTPSSSTPQENTISGYSLLTCKRTLPEPCHSKIYPGVDFGGEELNVTFVKGVNVCQ |
| | ETCTKMIRCQFFTYSLLPEDCKEEKCKCFLRLSMDGSPTRIAYGTQGSSGYSLRLCNTGD |
| | NSVCTTKTSTRIVGGTNSSWGEWPWQVSLQVKLTAQRHLCGGSLIGHQWVLTAAHCFDGL |
| | PLQDVWRIYSGILNLSDITKDTPFSQIKEIIIHQNYKVSEGNHDIALIKLQAPLNYTEFQ |
| | KPICLPSKGDTSTIYTNCWVTGWGFSKEKGEIQNILQKVNIPLVTNEECQKRYQDYKITQ |
| | RMVCAGYKEGGKDACKGDSGGPLVCKHNGMWRLVGITSWGEGCARREQPGVYTKVAEY |
| | MDWILEKTQSSDGKAQMQSPAEQKLISEEDLGGEQKLISEEDLHHHHHH |
| 48 | MILFKQATYFISLFATVSCGCLTQLYENAFFRGGDVASMYTPNAQYCQMRCTFHPRCLLF |
| | SFLPASSINDMEKRFGCFLKDSVTGTLPKVHRTGAVSGHSLKQCGHQISACHRDIYKGVD |
| | MRGVNFNVSKVSSVEECQKRCTNNIRCQFFSYATQTFHKAEYRNNCLLKYSPGGTPTAIK |
| | VLSNVESGFSLKPCALSEIGCHMNIFQHLAFSDVDVARVLTPDAFVCRTICTYHPNCLFF |
| | TFYTNVWKIESQRNVCLLKTSESGTPSSSTPQENTISGYSLLTCKRTLPEPCHSKIYPGV |
| | DFGGEELNVTFVKGVNVCQETCTKMIRCQFFTYSLLPEDCKEEKCKCFLRLSMDGSPTRI |
| | AYGTQGSSGYSLRLCNTGDNSVCTTKTSTRIVGGTNSSWGEWPWQVSLQVKLTAQRHLCG |
| | GSLIGHQWVLTAAHCFDGLPLQDVWRIYSGILNLSDITKDTPFSQIKEIIIHQNYKVSEG |
| | NHDIALIKLQAPLNYTEFQKPICLPSKGDTSTIYTNCWVTGWGFSKEKGEIQNILQKVNI |
| | PLVTNEECQKRYQDYKITQRMVCAGYKEGGKDACKGDSGGPLVCKHNGMWRLVGITSWGE |
| | GCARREQPGVYTKVAEYMDWILEKTQSSDGKAQMQSPA |

We claim:

1. An isolated antibody, or antigen-binding fragment thereof, that binds to the catalytic domain (CAT) domain of coagulation factor XI (FXI), wherein the antibody, or antigen-binding fragment thereof, comprises
a heavy chain (HC), comprising a heavy chain variable region (HCVR) comprising a heavy chain complementarity determining region (HCDR) 1, HCDR2, and HCDR3, wherein the HCDR1, HCDR2, and HCDR3 sequences are HCDR1, HCDR2, and HCDR3 sequences within SEQ ID NO: 18; and
a light chain (LC) comprising a light chain variable region (LCVR) comprising a light chain complementarity determining region (LCDR) 1, LCDR2, and LCDR3, wherein the LCDR1, LCDR2, and LCDR3 sequences comprise LCDR1, LCDR2, and LCDR3 sequences within SEQ ID NO: 20.

2. The isolated antibody, or antigen-binding fragment thereof, of claim 1, wherein the HCVR comprises an amino acid sequence having at least 90% identity to a HCVR sequence of SEQ ID NO: 2, and wherein the LC comprises an amino acid sequence having at least 90% identity to an LCVR sequence of SEQ ID NO: 10.

3. The isolated antibody, or antigen-binding fragment thereof, of claim 2, wherein the HC comprises an amino acid sequence comprising SEQ ID NO: 18, and wherein the LC comprises an amino acid sequence comprising SEQ ID NO: 20.

4. An isolated antibody, or antigen-binding fragment thereof, that binds to the catalytic (CAT) domain of coagulation factor XI (FXI), wherein the antibody, or antigen-binding fragment thereof, comprises a heavy chain region (HC) comprising an amino acid sequence having at least 90% identity to SEQ ID NO: 18 and a light chain region (LC) comprising an amino acid sequence having at least 90% identity to SEQ ID NO: 20.

5. The isolated antibody, or antigen-binding fragment thereof, of claim 4, wherein the HC comprises an amino acid sequence having at least 95% identity to SEQ ID NO: 18, and wherein the LC comprises an amino acid sequence having at least 95% identity to SEQ ID NO: 20.

6. The isolated antibody, or antigen-binding fragment thereof, of claim 4, wherein the HC comprises an amino acid sequence comprising SEQ ID NO: 18, and wherein the LC comprises an amino acid sequence comprising SEQ ID NO: 20.

7. The isolated antibody or antigen-binding fragment of claim 1, wherein the antibody, or antigen-binding fragment thereof, binds human FXI with a K_{D} of less than about 5 pM as measured by surface plasmon resonance at 25° C. or 37° C.

8. The isolated antibody or antigen-binding fragment of claim 7, wherein the antibody, or antigen-binding fragment thereof, binds human FXI with a K_{D} selected from the group consisting of less than about 800 pM, less than about 500 pM, less than about 100 pM, or less than about 50 pM as measured by surface plasmon resonance at 25° C. or 37° C.

9. The isolated antibody or antigen-binding fragment of claim 1, wherein the antibody, or antigen-binding fragment thereof, binds human FXI with a dissociative half-life (t½) selected from the group consisting of greater than about 10 minutes, greater than about 60 minutes, greater than about 500 minutes, or greater than about 1,000 minutes as measured by surface plasmon resonance at 25° C. or 37° C.

10. The isolated antibody or antigen-binding fragment of claim 1, wherein the antibody, or antigen-binding fragment thereof, inhibits activation of human coagulation factor X (FX) with an IC₅₀ of less than about 10 nM.

11. The isolated antibody or antigen-binding fragment of claim 1, wherein the antibody, or antigen-binding fragment thereof, inhibits activation of human coagulation factor X (FX) with an IC₅₀ of less than about 40 pM.

12. The isolated antibody or antigen-binding fragment of claim 1, wherein the antibody, or antigen-binding fragment thereof, increases by at least 2.5-fold activated partial thromboplastin time (aPTT).

13. The isolated antibody or antigen-binding fragment of claim 12, wherein the antibody, or antigen-binding fragment thereof, does not increase prothrombin time (PT).

14. The isolated antibody or antigen-binding fragment of claim 1, wherein the antibody, or antigen-binding fragment thereof, inhibits FXIa-mediated thrombin activity by at least 5%, by at least 10%, by at least 15%, or by 5%-15%.

15. The isolated antibody or antigen-binding fragment of claim 1, wherein the antibody, or antigen-binding fragment thereof, prolongs aPTT by at least two-fold in human plasma at a concentration ≤100 nM, ≤75 nM, or ≤50 nM.

16. The isolated antibody or antigen-binding fragment of claim 15, wherein the antibody, or antigen-binding fragment thereof, does not prolong PT.

17. The isolated antibody, or antigen-binding fragment thereof, of claim 1, wherein the antibody, or antigen-binding fragment thereof, inhibits thrombin production or activation via the intrinsic coagulation pathway in human plasma at a concentration of at least 10 nM, at least 25 nM, or at least 50 nM without affecting thrombin production or activation via the extrinsic coagulation pathway.

18. An isolated antibody, or antigen-binding fragment thereof, which competes for binding with the antibody, or antigen-binding fragment thereof, of claim 1.

19. An isolated antibody, or antigen-binding fragment thereof, which binds the same epitope as the antibody, or antigen-binding fragment thereof, of claim 1.

20. A pharmaceutical composition comprising the antibody, or antigen-binding fragment thereof, of claim 1, and a pharmaceutically acceptable carrier or diluent.

21. An isolated nucleic acid molecule comprising a polynucleotide sequence that encodes the antibody, or antigen-binding fragment thereof, according to claim 1.

22. A vector comprising the nucleic acid molecule of claim 21.

23. A cell comprising the vector of claim 22.

24. A method for inhibiting a biological activity mediated by FXI, the method comprising:
contacting FXI or FXIa with a biologically effective amount of the antibody, or antigen-binding fragment thereof, of claim 1.

25. The method of claim 24, wherein the biological activity is thrombogenesis, and wherein thrombogenesis is inhibited upon contact of FXI with the antibody, or antigen-binding fragment thereof.

26. The method of claim 25, wherein the contact results in prolonging aPTT or reducing thrombin activity in plasma.

27. A method of treating or preventing a disease or disorder associated with FXI activity or expression, or for ameliorating at least one symptom associated with the disease or disorder associated with FXI activity or expression, in a subject the method comprising administering a therapeutically effective amount of the antibody, or antigen-binding fragment thereof, of claim 1 to a subject in need of such treatment.

28. The method of claim 27, wherein the disease or disorder is a disease or disorder of blood coagulation or a disease or disorder that provides an increased risk of thrombogenesis in the subject.

29. The method of claim 28, wherein the disease or disorder is atrial fibrillation.
